(19) United States
(12) Patent Application Publication
(10) Pub. No.: US 2023/0100559 A1
(43) Pub. Date: Mar. 30, 2023

(54) Compositions and Methods for Treatment of Disease by Manipulation of Serine Metabolism

(71) Applicant: The Trustees of Princeton University, Princeton, NJ (US)

(72) Inventors: Joshua D. Rabinowitz, Princeton, NJ (US); Lifeng Yang, Princeton, NJ (US)

(21) Appl. No.: 17/790,702

(22) PCT Filed: Jan. 7, 2021
(86) PCT No.: PCT/US2021/012516

§ 371 Date: Jul. 1, 2022

Related U.S. Application Data
(60) Provisional application No. 62/958,116, filed on Jan. 7, 2020.

(51) Int. Cl. A61K 31/155; A61K 31/454; A61K 31/352; A61K 31/365; A61P 9/10

(52) U.S. Cl. CPC A61K 31/155; A61K 31/454; A61K 31/352; A61K 31/365; A61P 9/10

(57) ABSTRACT

The present disclosure relates, in various embodiments, to methods of treating various NADH-mediated diseases and disorders in a subject, such as disorders characterized by excess NADH, for example, by inhibiting serine catabolism in the subject.

### RELATED APPLICATION

This application claims the benefit of U.S. Provisional Application No. 62/958,116, filed on Jan. 7, 2020. The entire teachings of this application are incorporated herein by reference.

### GOVERNMENT SUPPORT

This invention was made with government support under Grant Nos: DK113643 and CA163591 awarded by the National Institutes of Health. The government has certain rights in the invention.

### BACKGROUND

NADH provides high energy electrons to the electron transport chain (ETC) for ATP production. In cells deprived of oxygen or with impaired ETC activity, NADH accumulation can be toxic. In such conditions, mammalian cells typically downregulate TCA metabolism and associated NADH production. However, in certain disease states, NADH levels remain at undesirably high levels.

Accordingly, there is a need for compositions and methods for manipulating NADH levels in NADH-related disorders.

### SUMMARY

The present disclosure generally relates to compositions and methods for manipulating serine catabolism for treating NADH-related disorders, such as, e.g., respiration impairment.

In one aspect, the disclosure provides methods of treating disorders associated with excess NADH, such as, e.g., respiration impairment, in a subject (e.g., a subject in need thereof), comprising administering an effective amount of an inhibitor of serine metabolism to the subject.

In one aspect, the disclosure provides methods of treating disorders associated with excess NADH, such as, e.g., respiration impairment, in a subject (e.g., a subject in need thereof), comprising administering an effective amount of an inhibitor of serine catabolism to the subject.

In some embodiments, the disorder is caused by hypoxia. In some embodiments, the disorder is caused by an ischemia. In some embodiments, the disorder is caused by a tumor. In some embodiments, the disorder is caused by impaired oxygenation due to excess adipose tissue. In some embodiments, the disorder is caused by an ischemic event.

In other embodiments, the respiration impairment is caused by a genetic deficiency of the electron transport chain. In other embodiments, the respiration impairment is caused by Leigh syndrome.

In some embodiments, the inhibitor of serine catabolism is an antifolate.

In some embodiments, the inhibitor comprises a compound that inhibits (e.g., blocks) SHMT2, MTHFD2 or a combination thereof. In one embodiment, the inhibitor is KDG112.

In some embodiments, the subject has received an electron transport chain inhibitor.

In another aspect, the disclosure provides methods of treating a disorder associated with excess NADH, such as, e.g., respiration impairment, in a subject (e.g., a subject in need thereof), comprising modulating serine metabolism in the subject.

In another aspect, the disclosure provides methods of treating a disorder associated with excess NADH, such as, e.g., respiration impairment, in a subject (e.g., a subject in need thereof), comprising modulating serine catabolism in the subject.

In some embodiments, the respiration impairment is caused by a genetic deficiency of the electron transport chain. In further embodiments, the respiration impairment is caused by Leigh syndrome.

In some embodiments, the modulating comprises inhibiting serine catabolism.

In some embodiments, the disorder is caused by hypoxia (e.g., anoxia). In some embodiments, the disorder is caused by an ischemia. In some embodiments, the disorder is caused by a tumor. In some embodiments, the disorder is caused by impaired oxygenation due to excess adipose tissue. In some embodiments, the disorder is caused by an ischemic event.

In a further aspect, the disclosure provides methods of treating a mitochondrial disorder in a subject (e.g., a subject in need thereof), comprising administering a composition that modulates the mitochondrial serine catabolic pathway. In some embodiments, the composition comprises a compound that inhibits SHMT2, MTHFD2 or a combination thereof. In some embodiments, the composition comprises KDG112.

In another aspect, the disclosure provides a method of treating a mitochondrial disorder in a subject (e.g., a subject in need thereof), comprising administering an effective amount of an inhibitor of serine metabolism (e.g., an inhibitor of serine catabolism) to the subject, wherein the inhibitor of serine metabolism inhibits mitochondrial serine metabolism (e.g., mitochondrial serine catabolism). In some embodiments, the inhibitor of serine metabolism is an inhibitor of SHMT2 and/or MTHFD2.

In another aspect, the disclosure provides methods for treating a disease involving deficient respiration in a subject (e.g., a subject in need thereof), comprising administering a therapeutically effective amount of an inhibitor of serine metabolism to the subject.

In some embodiments, the disease is a genetically inherited mitochondrial disorder. In some embodiments, the disease is myocardial infarction or stroke.

In some embodiments, the inhibitor of serine metabolism is an antifolate.

In some embodiments, the inhibitor inhibits SHMT2, MTHFD2 or a combination thereof.

In another aspect, the disclosure provides methods for treating a mitochondrial disease in a subject (e.g., a subject in need thereof), comprising administering a diet or dietary supplement deficient in serine compared to other amino acids to the subject.

In another aspect, the disclosure provides a method of improving the therapeutic index of an electron transport chain inhibitor (e.g., metformin, IACS-10759) in a subject (e.g., a subject in need thereof), comprising administering to the subject a therapeutically effective amount of an inhibitor of serine metabolism (e.g., an inhibitor of serine catabolism), wherein the subject is receiving the electron transport chain inhibitor in combination with the inhibitor of serine metabolism.

In another aspect, the disclosure provides methods of improving the therapeutic index of metformin in a subject (e.g., a subject in need thereof), comprising administering a therapeutically effective amount of an inhibitor of serine metabolism to the subject.

In a further aspect, the disclosure provides a method for improving the effectiveness of an electron transport chain inhibitor (e.g., metformin, IACS-10759) in a subject (e.g., a subject in need thereof), comprising administering to the subject an effective amount of a serine supplement, wherein the subject is receiving the electron transport chain inhibitor in combination with the serine supplement.

In a further aspect, the disclosure provides methods for improving the effectiveness of metformin in a subject (e.g., a subject in need thereof), comprising administering a serine supplement to the subject.

In another aspect, the disclosure provides methods of treating a tumor in a subject (e.g., a subject in need thereof), comprising administering to the subject an effective amount of an activator of serine catabolism. In one embodiment, the method further comprises administering an electron transport chain inhibitor to the subject. The electron transport chain inhibitor can be metformin, phenformin, rotenone, deguelin, antimycin, oligomycin, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings.

FIGS. 1A-1G show that serine catabolism feeds NADH in vivo. FIG. 1A: Schematic showing folate-mediated serine catabolism and its tracing with [2,3,3-²H]serine. This tracer has traditionally been used to distinguish cytosolic versus mitochondrial production of 1C units from serine, with the cytosolic pathway yielding M+2 thymidine and the mitochondrial pathway yielding M+1 thymidine. Smaller filled circles indicate labeled hydrogens (deuteriums). The cytosolic pathway (blue) yields M+2 thymidine and the mitochondrial pathway (red) yields M+1 thymidine. FIG. 1B: [2,3,3-²H]serine labels malate in vivo. Data are fractional ²H-labeling of malate relative to tissue serine labeling (fraction of M+2 and M+3 ²H-serine) after 2.5 h [2,3,3-²H]serine infusion (40 nmoles/g/mins). (mean±SD, N≥3). Throughout, all reported labeling fractions are corrected for natural isotope abundance. FIG. 1C: Raw orbitrap mass spectrum of M+1 malate in pancreas after [2,3,3-²H]serine infusion, showing the ²H peak well-resolved from the natural abundance ¹³C peak. FIG. 1D: Two possible metabolic pathways by which [2,3,3-²H]serine can produce ²H-malate. FIG. 1E: Raw mass spectrum of M+1 pyruvate in pancreas after [2,3,3-²H]serine infusion. Lack of pyruvate labeling rules out serine labeling malate via the pyruvate pathway. FIG. 1F: [2,3,3-2H]serine labels NADH more than malate. Data are fractional 2H-labeling of malate relative to tissue serine labeling (mean±SD, N≥4). FIG. 1G: Raw FTICR mass spectrum of M+1 NADH in pancreas and heart after [2,3,3-2H]serine infusion. Heart shows only one major peak: the natural abundance ¹³C-peak. Pancreas shows three peaks: an interfering peak of unknown identity, the natural abundance ¹³C-peak, and a partially resolved ²H-peak.

FIGS. 2A-2F show that serine catabolism is a major NADH source when respiration is impaired. FIG. 2A: Schematic of mitochondrial NADH sources. FIG. 2B: Fractional ²H-labeling of NADH from the indicated ²H-labeled glutamine (gln), lactate (lac), palmitate (pal), or serine (ser). HCT116 cells were grown in standard DMEM+5 mM metformin with the indicated nutrient replaced by its ²H-labeled form (except for palmitate, for which 100 μM was added to standard DMEM) (mean±SD, N=4). FIG. 2C: Oxygen consumption rate and fraction NAD²H from [2,3,3-²H] serine as a function of metformin concentration (mean±SD, N≥4). FIG. 2D: NAD²H from [2,3,3-2H]serine is via the mitochondrial folate pathway (mean±SD, N=3). FIG. 2E: Fraction NAD²H from [2,3,3-²H] serine in HCT116 cells (mean±SD, N=3). FIG. 2F: Fraction NAD²H from [2,3,3-²H] serine in 293T cells (mean±SD, N=3). ND, none detected; **p<0.01; ***p<0.001 by two-tailed student t test.

FIGS. 3A-3E show that NADH/NAD+ ratio dictates serine's NADH contribution. FIG. 3A: Schematic of electron transport chain. FIG. 3B: Fraction NAD²H from [2,3,3-²H]serine in the presence of the 1 μM rotenone, 500 nM atpenin, 1 μM antimycin, or 1 μM M oligomycin or electron transport chain deficient cells (ρ0) (mean±SD, N≥3). FIG. 3C: Correlation of fraction NAD²H from [2,3,3-²H] serine with intracellular NADH/NAD ratio (relative to DMEM without metformin). FIG. 3D: Duroquinone (DQ) and pyruvate can serve as electron acceptors to convert NADH into NAD. FIG. 3E: Impact of DQ and pyruvate on NADH/NAD+ and the fraction NAD²H from [2,3,3-²H] serine in HCT116 cells cultured with 5 mM metformin (mean±SD, N≥4). ** indicates p<0.01, *** indicates p<0.001 for two tail student t test.

FIGS. 4A-4F show that high NADH/NAD+ shuts off other NADH sources but not serine catabolism. FIG. 4A: Decrease in [U-¹³C]glucose entry into TCA cycle with increasing cellular NADH/NAD+. FIG. 4B: Decrease in [U-¹³C]glutamine oxidation (glutamine is instead metabolized reductively, see Supplementary FIG. 11D). FIG. 4C: Initial increase, followed by decrease, in [U-¹³C]palmitate entry into TCA cycle. FIG. 4D: Maintenance of mitochondrial serine catabolism indicated by M+1 dTTP from [2,3,3-²H]serine (see FIG. 1A). FIG. 4E: Quantification of intracellular NAD(H) concentration with metformin treatment (the increase in NADH/NAD ratio with metformin treatment is largely driven by NADH accumulation). FIG. 4F: Michaelis-Menten plot of pyruvate dehydrogenase (PDH), α-ketoglutarate dehydrogenase (KGDH), malate dehydrogenase (MDH) and MTHFD2 enzyme activity with increasing NADH as the competitive inhibitor (mean±SD, N=3).

FIGS. 5A-5F show that loss of mitochondrial serine catabolism paradoxically facilitates cell growth in respiration-impaired cells. FIG. 5A: NADH/NAD for wild type and MTHFD2 knockout HCT116 cells±metformin (mean±SD, N≥6). FIG. 5B: Schematic of links between NADH/NAD, aspartate, and nucleotide synthesis. FIGS. 5C-E: Intracellular aspartate (FIG. 5C), IMP/AMP ratio (FIG. 5D) and cell number (FIG. 5E) for wild type and MTHFD2 deficient HCT116 in the presence and absence of the indicated concentration of metformin (mean±SD, N≥6). FIG. 5F: HCT116 cell growth in normal medium or the presence of 1 mM metformin the indicated concentration of the serine catabolism inhibitor SHIN1 (mean±SD, n=5). **p<0.01, ***p<0.001 by two-tailed Student's t test.

FIGS. 6A-6I show that hypoxia induces serine-dependent NADH production. FIG. 6A: NADH/NAD+ in normoxia and hypoxia (mean±SD, N≥8). FIG. 6B: Fraction NAD²H from ²H-labeled nutrients in wild-type HCT116 cells in normoxia and hypoxia (tracers as in FIG. 2A, mean±SD, N≥3). FIG. 6C: ²H-labeled metabolites (M+1) from [2,3,3-²H]serine in normoxia and hypoxia (mean±SD, N=6). D: Intracellular aspartate in hypoxia (0.5% O₂) (mean±SD, N=6). FIG. 6E-6F: Relative IMP/AMP ratio (E) (mean±SD, N=6) and cell number (F) (mean SEM, N≥14) for wild type and MTHFD2-knockout HCT116 in normoxia and hypoxia, p-values refer to difference between knockout and WT in hypoxia. FIG. 6G: Relative L-2 hydroxyglutarate level for HCT116 cell lines in normoxia and hypoxia (mean±SD, N=9). FIG. 6H: HCT116 WT cell growth in hypoxia (0.5% O2)±the indicated concentrations of the serine catabolism inhibitor SHIN1 (mean±SD, N=5). P value indicates statistical analysis between WT and KO (G) or no drug versus 62.5 nM or 250 nM SHIN1 (H). FIG. 6I: Relative L-2 hydroxyglutarate level for HCT116 cell lines in normoxia and hypoxia (mean±SD, n=9). * p<0.05, **p<0.01. ***p<0.001 by two-tailed student t test.

FIGS. 7A-7F show that serine catabolism provides NADH in fibroblasts derived from Leigh Syndrome patients and NDUFS4−/− mice. FIGS. 7A-7C: NADH/NAD+, fraction NAD²H from [2,3,3-²H]serine and fraction ²H-malate from [2,3,3-²H]serine in cultured fibroblasts from healthy donors and Leigh Syndrome patients (mean±SD, N=3 for heathy donors, N=5 from Leigh Syndrome patients). FIG. 7D: Increased malate labeling from [2,3,3-2H]serine in NDUFS4−/− mice in vivo. Data are fractional 2H-malate labeling relative to tissue serine labeling after 2.5 h [2,3,3-2H] serine infusion (40 nmoles/g/min) (mean±SD, N=6 for WT, N=9 for NDUFS4−/−). FIG. 7E: SHIN2 blocks NADH production by serine catabolism in vivo. WT C57BL6/J mice were treated with SHIN2 (200 mg/kg, ip) immediately before a 2.5 h [2,3,3-2H]serine infusion (mean±SD, N=3). FIG. 7F: Delayed occurrence of limb clasping in NDUFS4−/− treated with SHIN2 (N=7 for vehicle treatment, N=9 for SHIN2 treatment). *p<0.05, **p<0.01 with two-tailed student t test.

FIGS. 8A-8F. Serine catabolism feeds NADH in vivo. FIG. 8A: Raw mass spectrum of M+1 malate, M+1 ²H pyruvate and M+2 ²H pyruvate in KPC pancreatic tumor after [2,3,3-²H] serine infusion. FIG. 8B: Raw FTICR mass spectrum of M+1 NAD in pancreas and heart after [2,3,3-2H]serine infusion. FIG. 8C: Schematic of the conversion of U-¹³C serine into TCA cycle metabolites by serine dehydratase (SDS). FIG. 8D: [2,3,3-2H]serine labels malate more than aspartate. Data are fractional 2H-labeling of aspartate relative to tissue serine labeling (mean±SD, N≥3). FIG. 8E: Relative expression level of MTHFD2 across different tissues. FIG. 8F: Pig arteriovenous serine differences for different tissues. Data are log 2 of venous serine abundance relative to arterial serine abundance across the indicated tissue. Positive values indicate serine secretion and negative values indicate serine uptake (N=10, reflecting 2 blood draws from each of 5 pigs).

FIGS. 9A-9D. Serine catabolism is a major NADH source when respiration is impaired. FIG. 9A: Schematic showing glutaminolysis and its tracing with [2,3,3,4,4-²H]glutamine. This tracer will generate NAD2H following the enzymatic activities of glutamate dehydrogenase and malate dehydrogenase. FIG. 9B: Schematic showing lactate into TCA cycle and its tracing with [3,3,3-²H] lactate. This tracer will generate NAD2H by enzymatic activity of isocitrate dehydrogenase (2H is from oxaloacetate, which is derived from [3,3,3-²H] pyruvate via pyruvate carboxylase) and by enzymatic activity of malate dehydrogenase (2H is from acetyl-CoA, which is derived from [3,3,3-2H] pyruvate via pyruvate dehydrogenase). FIG. 9C: Oxygen consumption rate and fraction NAD²H from [2,3,3-2H] serine as a function of phenformin concentration (mean±SD, N≥3). FIG. 9D: NAD2H from [2,3,3-2H] serine is via the mitochondrial folate pathway, proving by using 293T cell lines (mean±SD, N=2).

FIGS. 10A-10F. NADH/NAD+ ratio dictates serine's NADH contribution. FIG. 10A: Relative NADH/NAD+ in the presence of the 1 μM rotenone, 500 nM atpenin, 1 μM antimycin, or 1 μM oligomycin or electron transport chain deficient cells (ρ0) (mean±SD, N≥3). FIG. 10B: Atpenin treatment successfully blocks Complex II as indicated by succinate accumulation (mean±SD, N=5). C: NADH/NAD ratio and fraction NAD²H from [2,3,3-²H]serine as a function of metformin treatment duration (mean±SD, N≥3). FIGs. D, E and F: Mitochondrial pyruvate carrier inhibitor (UK5099) does not block pyruvate rescue of NADH phenotypes (mean±SD, N≥5). ** p<0.01, *** p<0.001 by two-tailed student's t-test.

FIGS. 11A-11J. High NADH/NAD+ shuts off other NADH sources but not serine catabolism. FIGS. 11A-11C: Schematic showing U-¹³C glucose (A), U-¹³C glutamine (B), U-¹³C palmitate (C) are catabolized into TCA cycle metabolites as well as generating NADH at the function of different dehydrogenases. FIG. 11D: Increase in [U-¹³C]glutamine reductive carboxylation. FIG. 11E: Initial increase, followed by decrease, in [U-¹³C]palmitate entry into TCA cycle, indicated from M+2 α-ketoglutarate. FIG. 11F: Correlation of intracellular NADH and NAD concentration with NADH/NAD+ ratio, increased by different concentrations of metformin treatment. FIG. 11G: Decrease in [U-¹³C]glucose entry into TCA cycle with increasing cellular NADH/NAD. FIG. 11H: Decrease in [U-¹³C] glutamine oxidation. FIG. 11I: Malate labeling from [U-¹³C]palmitate confirms that the fractional contribution of fatty acid oxidation to TCA initially increases, and then decreases, as NADH/NAD rises. FIG. 11J: De novo serine synthesis from [U-¹³C]glucose.

FIG. 12. Higher NADH inhibitory constant for MTHFD2 compared to MDH. FIG. 12: Relative cell number with different concentrations of metformin treatment for wild type and MTHFD2 deficient HCT116. Michaelis-Menten plot of malate dehydrogenase (MDH) and MTHFD2 enzyme activity with increasing NADH as the competitive inhibitor (mean±SD, N=3).

FIGS. 13A-13H. Loss of mitochondrial serine catabolism paradoxically facilitates cell growth in respiration-impaired cells. FIG. 13A: NADH/NAD for wild type and MTHFD2 knockout HCT116 cells±10 μM phenformin or 50 nM rotenone (mean±SD, N=3). FIG. 13B: Aspartate isotopic forms abundance in HCT116 cells fed [U-¹³C]glutamine plus the indicated respiratory chain inhibitors (mean±SD, N≥3). FIGS. 13C, 13D and 13E: MTHFD2 knock out enhances growth in the presence of metformin (mean±SD, N=6) (C), phenformin (mean±SD, N=5) (D) and rotenone (mean±SD, N=5) (E). FIG. 13F: Removal of serine from the medium enhances growth in the presence of metformin (mean±SD, N=3). FIG. 13G: The serine catabolism inhibitor SHIN1 enhances growth in the presence of metformin. Each line reflects a different concentration of SHIN1 (mean±SD, N=5). Statistical comparisons are for zero vs. 250 nM SHIN1. FIG. 13H: The serine catabolism inhibitor SHIN1 enhances growth in the presence of phenformin or rotenone (mean±SD, N=5). *p<0.05, **p<0.01, ***p<0.001 by two-tailed student's t test.

FIGS. 14A-14F. Impact of genetic respiratory chain deficiency on glucose, glutamine and serine metabolism. FIG. 14A: Fraction NAD²H from [2,3,3-²H]serine and fraction ²H-malate from [2,3,3-²H]serine in cultured and xenograft implanted wild type and MTO1 deficient HCT116. (mean±SD, N=3). FIG. 14B: intracellular aspartate for MTO1 deficient cells±the indicated concentration of the serine catabolism inhibitor SHIN1 (mean±SD, N=3). FIG. 14C: MTO1 deficient HCT 116 growth rate in normal DMEM medium±the indicated concentration of the serine catabolism inhibitor SHIN1 (mean±SD, N≥4). FIG. 14D: Circulatory turnover flux of glucose and glutamine in NDUFS4 deficient mice (mean±SD, N=4). FIG. 14E: Normalized labelling of tissue TCA intermediates after 2.5 h infusion of U-¹³C-glucose (20 nmoles/min/gram) (mean±SD, N=3) or U-¹³C-glutamine (10 nmoles/mins/gram) (mean±SD, N=3). FIG. 14F: Glucose and glutamine metabolic fate within the TCA cycle is similar across WT and NDUFS4 deficient mice, as measured based on M+2/M+3 malate from glucose (reflecting pyruvate entry to TCA via PDH versus PC) and M+3/M+4 malate from glutamine (reflecting α-ketoglutarate reductive carboxylation versus oxidation) (mean±SD, N=4).** indicates p<0.01 for two tail student t test.

FIGS. 15A-15E. Serine catabolism provides NADH in NDUFS4 deficient T cells and C2C12 cells treated with metformin. FIG. 15A: NADH/NAD ratio and fraction ²H-Malate from [2,3,3-²H]serine for T cells extracted from wild type and NDUFS4−/− mice and activated in vitro (mean±SD, N=3). FIGS. 15B, 15C and 15D: Serine catabolism inhibition (SHIN1, 250 nM) in C2C12 cells treated with 5 mM metformin enhances aspartate (B) (mean±SD, N=3), restores IMP/AMP ratio (C) (mean±SD, N=3), and cell growth (D) (mean±SD, N=5). FIG. 15E: Survival analysis for NDUFS4−/− mice with SHIN2 treatment versus vehicle (p=0.18, log-rank Test). *p<0.05, **p<0.01, ***p<0.001 by two-tailed student's t test.

FIGS. 16A-16C (**p<0.01, ***p<0.001 by two-tailed Student's t test). FIG. 16A: IACS-10759, a complex I inhibitor, increased intra-tumor lactate level of HCT116 xenograft tumors (HCT116 colon cancer cell lines were xenografted on the flank of CD-1 nude mice. IACS-10759 was dissolved in 90% corn oil, and the drug was gavaged at a dosage of 10 mg/kg every day for 7 consecutive days. Tumors were collected for metabolomics analysis), mean±SD, n=6. FIG. 16B: IACS-10759 increased NADH/NAD of HCT116 xenograft tumors. IACS-10759 can increase reductive stress in vivo as mitochondrial complex I inhibitor, mean±SD, n=6. FIG. 16C: IACS-10759 increased ²H-malate labeling from [2,3,3-²H]serine in HCT116 xenograft tumors. Data are fractional ²H-malate relative to tissue serine labeling after 2.5 hours [2,3,3-²H]serine infusion (40 nmoles/g/min) (mean±SD, n=6).

FIG. 17: Serine/metformin in drinking water increased liver NADH/NAD ratio. Mice were given 5% serine or 0.25% metformin drinking water for 2 weeks. Liver tissues were collected for metabolomics analysis. mean±SD, n=6. *p<0.05, **p<0.01 via two-tailed Student's t test.

### DETAILED DESCRIPTION

A description of example embodiments follows.

### Definitions

Unless otherwise defined herein, scientific and technical terms used in this disclosure shall have the meanings that are commonly understood by those of ordinary skill in the art. Generally, nomenclature used in connection with, and techniques of, chemistry, cell and tissue culture, molecular biology, cell and cancer biology, immunology, and pharmacology, described herein, are those well known and commonly used in the art.

Chemistry terms used herein are used according to conventional usage in the art, for example as exemplified by “The McGraw-Hill Dictionary of Chemical Terms”, Parker S., Ed., McGraw-Hill, San Francisco, Calif. (1985).

All of the above, and any other publications, patents and published patent applications referred to in this disclosure are specifically incorporated by reference herein. In case of conflict, the present disclosure, including its specific definitions, will control.

It is to be understood that the present disclosure is not limited to particular embodiments described, as such may, of course, vary. It is also to be understood that the terminology used herein is for the purpose of describing particular embodiments only, and is not intended to be limiting.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

Any formula depicted herein is intended to represent a compound of that structural formula as well as certain variations or forms. For example, a formula given herein is intended to include a racemic form, or one or more enantiomeric, diastereomeric, or geometric isomers, or tautomeric forms, or a mixture thereof. Additionally, any formula given herein is intended to refer also to a solvate, such as a hydrate, solvate, or polymorph of such a compound, or a mixture thereof Δny formula given herein is intended to refer to amorphous and/or crystalline physical forms of the compound. The compounds described herein may be analytically pure, or a mixture in which the compound comprises at least 50%, at least 70%, at least 80%, at least 90%, at least 95%, or at least 98% by weight of the mixture.

In addition, where features or aspects of the embodiments of this disclosure are described in terms of Markush groups, those skilled in the art will recognize that embodiments described herein is also thereby described in terms of any individual member or subgroup of members of the Markush group. For example, if X is described as selected from the group consisting of bromine, chlorine, and iodine, claims for X being bromine and claims for X being bromine and chlorine are fully described.

The term “herein” refers to the entire disclosure.

The term “alkoxy” refers to an oxygen atom having an alkyl group attached thereto. Representative alkoxy groups include methoxy, ethoxy, propoxy, tert-butoxy and the like. In some embodiments, a straight chain or branched chain alkoxy has 30 or fewer carbon atoms, and preferably 20 or fewer, such as C₁-C₁₀ alkoxy, C₁-C₈ alkoxy, or C₁-C₆ alkoxy.

The term “alkenyl” refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In some embodiments, a straight chain or branched chain alkenyl has 30 or fewer carbon atoms, and preferably 20 or fewer, such as C₂-C₁₀ alkenyl or C₂-C₈ alkenyl.

The term “alkynyl” refers to an aliphatic group containing at least one triple bond and is intended to include both “unsubstituted alkynyls” and “substituted alkynyls,” the latter of which refers to alkynyl moieties having substituents replacing one or more hydrogens on one or more carbons of the alkynyl group. Such substituents may occur on one or more carbons that are included or not included in one or more triple bonds. Moreover, such substituents include all those contemplated for alkyl groups, as discussed below, except where stability is prohibitive. For example, substitution of alkynyl groups by one or more alkyl, carbocyclyl, aryl, heterocyclyl, or heteroaryl groups is contemplated. In some embodiments, a straight chain or branched chain alkynyl has 30 or fewer carbon atoms, and preferably 20 or fewer, such as C₂-C₁₀ alkynyl or C₂-C₈ alkynyl.

The term “alkyl” refers to a saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g., methyl, ethyl, n-propyl, i-propyl, n-butyl and n-pentyl. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In some embodiments, the chain has eight or fewer carbon (C₁-C₈) atoms. In other embodiments, the chain has six or fewer carbon (C₁-C₆) atoms in its backbone. A C₁-C₆ straight chained or branched alkyl group is also referred to as a “lower alkyl” group. Moreover, the term “alkyl” (or “lower alkyl”) as used throughout the disclosure, examples, and claims is intended to include both “unsubstituted alkyls” and “substituted alkyls”, the latter of which refers to alkyl moieties having substituents replacing a hydrogen on one or more carbons of the hydrocarbon backbone.

Such substituents can include, but not limited to, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety. It will be understood by those skilled in the art that the moieties substituted on the hydrocarbon chain can themselves be substituted, if appropriate. For instance, the substituents of a substituted alkyl may include substituted and unsubstituted forms of hydroxyl, halo, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “arylkyl”, as used herein, refers to an alkyl group substituted with one or more aryl groups.

The term “aryl”, as used herein, includes substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like.

The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The terms “amine” and “amino” are art-recognized and refer to both unsubstituted and substituted amines and salts thereof, e.g., a moiety that can be represented by

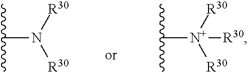

wherein each R³⁰ independently represents a hydrogen or a hydrocarbyl group, or two R³⁰ are taken together with the N atom to which they are attached complete a heterocycle having from 4 to 8 atoms in the ring structure.

The term “aminoalkyl” refers to an alkyl group substituted with an amino group.

The term “amide” refers to a group

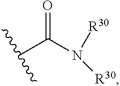

wherein each R³⁰ independently represent a hydrogen or hydrocarbyl group, or two R³⁰ are taken together with the N atom to which they are attached complete a heterocycle having from 4 to 8 atoms in the ring structure.

The terms “nitrile” or “cyano,” as used herein, refers to —CN.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “cycloalkyl”, as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated, and aromatic rings.

A “cycloalkenyl” group, as used herein, refers to a cyclic hydrocarbon containing one or more double bonds. A “cycloalkynyl” group is a cyclic hydrocarbon containing one or more triple bonds.

The terms “polycyclyl”, “polycycle”, and “polycyclic”, as used herein, refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The terms “halo” and “halogen”, as used herein, means halogen and includes chloro, fluoro, bromo, and iodo.

The term “haloalkyl”, as used herein, means an alkyl group substituted with one or more halogens. When more than one halogen is present, the halogens may be the same or different. For examples, haloalkyl groups include, but are not limited to, fluoromethyl, difluoromethyl, trifluoromethyl, chlorodifluoromethyl, 2,2,2-trifluoroethyl, pentafluoroethyl, and the like.

The term “haloalkoxy”, as used herein, means an alkoxy group substituted with one or more halogens. When more than one halogen is present, the halogens may be the same or different. For examples, haloalkyl groups include, but are not limited to, difluoromethoxy, trifluoromethoxy, 2,2,2-trifluoroethoxy, pentafluoroethoxy, and the like.

The term “heteroarylakyl”, as used herein, refers to an alkyl group substituted with a heteroaryl group.

The term “heteroaryl” includes substituted or unsubstituted aromatic single ring structures, preferably 5- to 7-membered rings, more preferably 5- to 6-membered rings, whose ring structures include at least one heteroatom (e.g., O, N, or S), preferably one to four, or one to 3 heteroatoms, more preferably one or two heteroatoms. When two or more heteroatoms are present in a heteroaryl ring, they may be the same or different. For examples, heteroaryl groups include, but are not limited to, pyrrole, furan, thiophene, imidazole, tetrazole, oxazole, thiazole, pyrazole, pyridine, pyrazine, pyridazine, pyrimidine, and the like. The term “heteroaryl” also include substituted or unsubstituted “polycyclic” ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls.

The term “heteroatom”, as used herein, means an atom of any element other than carbon or hydrogen. Exemplary heteroatoms include but are not limited to nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. In certain embodiments, the ring structure is saturated, such as heterocycloalkyls; in other embodiments, the ring structure is unsaturated, such as heterocycloalkenyls or heterocycloalkynyls. The terms “heterocyclyl” and “heterocyclic” also include substituted or unsubstituted polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. In certain embodiments, the ring structure can have two cyclic rings. In some embodiments, the two cyclic rings can have two or more atoms in common, e.g., the rings are “fused rings.” Heterocyclyl groups include, but no limited to, for example, piperidine, piperazine, pyrrolidine, morpholine, lactones, lactams, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Accordingly, substituents can further include an acetamide, for example.

Unless specifically stated as “unsubstituted,” references to chemical moieties herein are understood to include substituted variants. For example, reference to an “alkyl” group or moiety implicitly includes both substituted and unsubstituted variants. The term “unsubstituted” refers to that the specified group bears no substituents.

The term “optionally substituted”, as used herein, means that substitution is optional and therefore it is possible for the designated atom or moiety to be unsubstituted.

The term “ring” or “ring system”, unless context indicates otherwise, may include monocyclic rings or polycyclic rings, such as bicyclic rings. When the term ring refers to a polycyclic or bicyclic ring, each ring is independently selected from saturated or unsaturated, and either or both rings may contain one or more heteroatoms, preferably a total of 0, 1, 2, 3 or 4 heteroatoms across the ring system.

Compounds described herein can exist as various “solvates” or “hydrates.” A “hydrate” is a compound that exists in a composition with water molecules. The composition can include water in stoichiometic quantities, such as a monohydrate or a dihydrate, or can include water in random amounts. A “solvate” is a similar composition except that a solvent other that water, such as with methanol, ethanol, dimethylformamide, diethyl ether and the like replaces the water. For example, methanol or ethanol can form an “alcoholate,”” which can again be stoichiometic or non-stoichiometric. Mixtures of such solvates or hydrates can also be prepared. The source of such solvate or hydrate can be from the solvent of crystallization, inherent in the solvent of preparation or crystallization, or adventitious to such solvent.

The compounds described herein, including their pharmaceutically acceptable salts, can exist as various polymorphs, pseudo-polymorphs, or in amorphous state. The term “polymorph”, as used herein, refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus crystalline polymorphic forms are important aspects in the development of suitable dosage forms in pharmaceutical industry.

In certain embodiments, a “pharmaceutically acceptable” substance is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio. In certain embodiments, a “pharmaceutically acceptable” substance that is a component of a pharmaceutical composition is, in addition, compatible with the other ingredient(s) of the composition.

The terms “pharmaceutically acceptable excipient”, “pharmaceutically acceptable carrier” and “pharmaceutically acceptable diluent” encompass, without limitation, pharmaceutically acceptable inactive ingredients, materials, compositions and vehicles, such as liquid fillers, solid fillers, diluents, excipients, carriers, solvents and encapsulating materials. Carriers, diluents and excipients also include all pharmaceutically acceptable dispersion media, coatings, buffers, isotonic agents, stabilizers, absorption delaying agents, antimicrobial agents, antibacterial agents, antifungal agents, adjuvants, and so on. Except insofar as any conventional excipient, carrier or diluent is incompatible with the active ingredient, the present disclosure encompasses the use of conventional excipients, carriers and diluents in pharmaceutical compositions. See, e.g., Remington: The Science and Practice of Pharmacy, 21st Ed., Lippincott Williams & Wilkins (Philadelphia, Pa., 2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press LLC (Boca Raton, Fla., 2004). A “pharmaceutically acceptable salt” is a salt of a compound that is suitable for pharmaceutical use, including but not limited to metal salts (e.g., sodium, potassium, magnesium, calcium, etc.), acid addition salts (e.g., mineral acids, carboxylic acids, etc.), and base addition salts (e.g., ammonia, organic amines, etc.).

“Pharmaceutically acceptable salt” is used herein to refer to a compound according to the disclosure that is a therapeutically active, non-toxic base or acid salt form of the free base form of the compound. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like. See, e.g., WO 01/062726. Some pharmaceutically acceptable salts listed by Berge et al., Journal of Pharmaceutical Sciences, 66: 1-19 (1977), incorporated herein by reference in its entirety. Compounds described herein can be present as pharmaceutically acceptable salts of their free base form.

Compounds containing acidic protons may be converted into their therapeutically active, non-toxic base addition salt form, e.g., metal or amine salt, by treatment with appropriate organic and inorganic bases. Appropriate base salt forms include, for example, ammonium salts, alkali and earth alkaline metal salts, e. g., lithium, sodium, potassium, magnesium, calcium salts and the like, salts with organic bases, e. g. N-methyl-D-glucamine, hydrabamine salts, and salts with amino acids such as, for example, arginine, lysine and the like. Conversely, said salt forms can be converted into the free forms by treatment with an appropriate base or acid. Compounds and their salts can be in the form of a solvate, which is included within the scope of the present disclosure. Such solvates include for example hydrates, alcoholates and the like. See, e.g., WO 01/062726.

Other examples of pharmaceutically acceptable salts include, but are not limited to, camsylate, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17^{th} Edition, Mack Publishing Company, Easton, Pa., 1985.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The term “pharmaceutical composition” refers to a composition suitable for pharmaceutical use in a subject animal, including humans and mammals, e.g., combined with one or more pharmaceutically acceptable carriers, excipients or solvents. Such a composition may also contain diluents, fillers, salts, buffers, stabilizers, solubilizers, and other materials well known in the art. In certain embodiments, a pharmaceutical composition encompasses a composition comprising the active ingredient(s), and the inert ingredient(s) that make up the excipient, carrier or diluent, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound described herein and one or more pharmaceutically acceptable excipient(s), carrier(s) and/or diluent(s).

The disclosure further provides pharmaceutical compositions comprising one or more compounds described herein and a pharmaceutically acceptable excipient(s), carrier(s) and/or diluent(s). Compounds or pharmaceutical compositions of the disclosure may be used in vitro or in vivo.

Within the present disclosure it is to be understood that a compound described herein or a salt (e.g., pharmaceutically acceptable salt) thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae within this specification can represent only one of the possible tautomeric forms. However, it is also to be understood that the disclosure encompasses any tautomeric form, and is not to be limited merely to any one tautomeric form utilized within the formula drawings. The formula drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

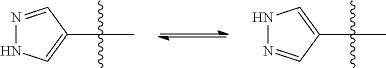 [SMILES: *c1cn[nH]c1 ; *c1cn[nH]c1]

Tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

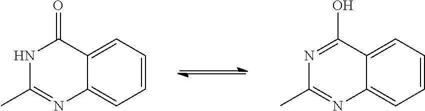

is an example of tautomerism. Accordingly, a structure depicted herein as one tautomer is intended to also include the other tautomer.

It will be understood that when compounds described herein contain one or more chiral centers, the compounds may exist in, and may be isolated as pure enantiomeric or diastereomeric forms or as mixtures of one or more enantiomers and/or diastereomers (e.g., racemic mixtures). The present disclosure includes all possible enantiomers and diastereomers, in their pure forms or as mixtures (e.g., racemic mixtures).

Isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

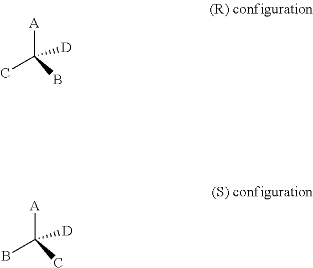

In certain embodiments, the therapeutic preparation may be enriched to provide predominantly one enantiomer of a compound (e.g., of formula (I), (Ia), or (Ib)). An enantiomerically enriched mixture may comprise, for example, at least 60 mol percent of one enantiomer, or more preferably at least 75, 90, 95, or even 99 mol percent. In certain embodiments, a compound of the disclosure may have greater than 30% ee, 40% ee, 50% ee, 60% ee, 70% ee, 80% ee, 90% ee, or even 95% or greater ee. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, compounds described herein may have more than one stereocenter. In certain such embodiments, the compounds may be enriched in one or more diastereomer. For example, a compound may have greater than 30% de, 40% de, 50% de, 60% de, 70% de, 80% de, 90% de, or even 95% or greater de.

Isolated optical isomers may be purified from racemic mixtures by well-known chiral separation techniques, such as but not limited to, normal and reverse phase chromatography, and crystallization. According to one such method, a racemic mixture of a compound, or a chiral intermediate thereof, is separated using a chiral salt or carried out on a Chiralcell OD column. The column can be operated according to the manufacturer's instructions.

Isolated optical isomers (enantiomerically pure compounds) can also be prepared using chiral intermediates or catalysts in synthesis. When a chiral synthetic intermediate is used, the optical center (chiral center) can be preserved without racemization throughout the remainder of the preparative procedure, as is well known in the art. Chiral catalyst can be used to impart at least some degree of enantiomeric purity to products of reactions catalyzed by the chiral catalyst. And, in some cases, compounds having at least some degree of enantiomeric enrichment can be obtained by physical processes such as selective crystallization of salts or complexes formed with chiral adjuvants.

A variety of compounds may exist in particular geometric or stereoisomeric forms. The present disclosure takes into account all such compounds, including tautomers, cis- and trans-isomers, R- and S-enantiomers, diastereomers, (D)-isomers, (L)-isomers, the racemic mixtures thereof, and other mixtures thereof, as being covered within the scope of this disclosure. All tautomeric forms are encompassed in the present disclosure. Additional asymmetric carbon atoms may be present in a substituent such as an alkyl group. All such isomers, as well as mixtures thereof, are intended to be included in this disclosure, unless the stereochemistry or isomeric form is specifically indicated.

For example, compounds of Formulae (I)-(IX) (and the other compounds of the disclosure) have one or more chiral centers and therefore can exist as enantiomers and/or diastereomers. Compounds of Formulae (I)-(IX) (and the other compounds of the disclosure) may also exist as stereoisomers, for example atropisomers, resulting from hindered rotation about a single bond. The compound of the disclosure are understood to extend to, and embrace all such enantiomers, diastereomers, atropisomers, stereoisomers, and mixtures thereof, including but not limited to racemates. Formulae (I)-(IX) (and the other compounds of the disclosure) used throughout this disclosure are intended to represent all individual stereoisomers and mixtures thereof, unless stated or shown otherwise.

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present disclosure therefore includes any possible stable rotamers of formula (I) which are biologically active in the treatment of cancer or other proliferative disease states.

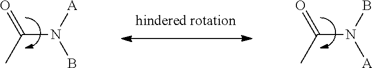

The compounds described herein have a particular spatial arrangement of substituents on the aromatic rings, which are related to the structure activity relationship demonstrated by the compound class. Often, such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

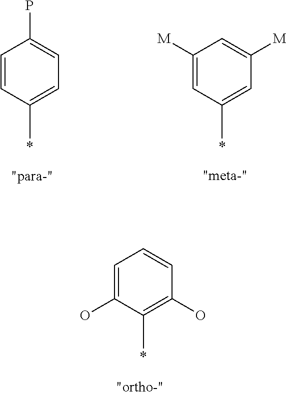

In some embodiments, the compounds described herein may contain a double bond. It is understood that cis/trans isomers are configurational isomers having different orientation at the double bond. In certain such embodiments, the compounds of this disclosure can be in either cis- or trans-formation. In the present disclosure, the term “cis” is equivalently used for “Z” and vice versa “trans” for ‘E” and vice versa.

Compounds described herein also include all isotopes of atoms occurring in the compounds. Isotopes include those atoms having the same atomic number but different mass numbers. For example, isotopes of hydrogen include deuterium and tritium.

As used herein, the term “subject” refers to an animal. The terms “subject” and “patient” may be used interchangeably herein in reference to a subject. The term “subject” includes mammals (e.g., humans, non-human primates, cows, sheep, goats, horses, dogs, cats, rabbits, guinea pigs, rats, mice, etc.). In particular embodiments, the subject is a human.

The terms “treat,” “treating” and “treatment” refer to the treatment of a patient afflicted with a pathological condition and refers to an effect that alleviates the condition, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

“SHMT” refers to serine hydroxymethyltransferase. Such enzymes are known and, in mammals, both SHMT1 and SHMT2 are expressed and active. Exemplary SHMTs include mammalian SHMT1 and SHMT2, such as human SHMT1 and SHMT2. Further structural information regarding human SHMT1 can be found at NCBI entrez ID number 6470. Further structural information regarding human SHMT2 can be found at NCBI entrez ID number 6472.

In certain embodiments, by “SHMT activity” is meant a native function of a mammalian SHMT enzyme, such its native enzymatic activity. In certain embodiments, SHMT activity refers to the function of mammalian SHMT to catalyze a reversible reaction converting serine to glycine. In certain embodiments, SHMT activity refers to the function of mammalian SHMT to catalyze a reversible reaction converting serine to glycine with concurrent methylene-tetrahydrofolate (meTHF) generation. In certain embodiments, SHMT activity refers to the generation of 1C units. SHMT activity may be assayed or evaluated in numerous ways, such as is described herein. SHMT activity may be evaluated by evaluating serine flux and/or folate metabolism, such as mitochondrial serine flux, glycine synthesis, NADPH generation, generation and excretion of formate or mitochondrial folate metabolism.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration.

Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity).

A “therapeutically effective amount” or “effective amount”, used interchangeably herein, refers to an amount effective, at dosages and for periods of time necessary, to achieve a desired therapeutic result. The full therapeutic effect does not necessarily occur by administration of one dose, and may occur only after administration of a series of doses. Thus, a therapeutically effective amount may be administered in one or more administrations. A therapeutically effective amount may vary according to factors such as the disease state, age, sex, and weight of the individual, and the ability of a therapeutic or a combination of therapeutics to elicit a desired response in the individual. A skilled worker can readily determine the effective amount for a given situation.

### Methods

The present disclosure generally relates to methods and related compositions for manipulating serine metabolism (e.g., serine catabolism) for treating NADH related disorders, such as, e.g., respiration impairment.

In cells deprived of oxygen or with impaired ETC activity, the cells downregulate TCA metabolism and associated NADH production. Under these conditions, folate-mediated serine catabolism becomes a major NADH source. Deuterium tracing revealed a substantial fractional serine contribution to NADH, mediated by the mitochondrial enzyme methylenetetrahydrofolate dehydrogenase (MTHFD2). This contribution increases several fold in response to hypoxia or pharmacological respiration inhibition. Under such conditions, blockade of mitochondrial serine catabolism partially normalizes the NAD/NADH ratio and promotes cell growth. In vivo, deuterium tracing revealed a meaningful contribution of serine catabolism to NADH in pancreas, spleen, liver and tumors. This contribution increased in mice with ETC deficiency.

Mammals break down carbohydrate, amino acids (glutamine, branch chain amino acids), and fatty acids for the energy generation. Through tricarboxylic acid (TCA) turning in mitochondria, these nutrients are burned into CO₂, as well as producing high energy reducing equivalent NADH. NADH is recycled back to its oxidative form NAD+ via mitochondrial complex I, driving high energetic electron to electron transport chain (ETC) for ATP generation. Electrons transferred from NADH to mitochondrial complex I and downstream components of the electron transport chain (ETC), eventually reduce oxygen to water. By inducing a proton gradient, these reactions power mitochondrial ATP synthase, which provides the bulk of ATP in mammals. These sequentially coordinated chemical reactions guarantee a redox homeostasis inside of mitochondria. A disruption of ETC function, either by pharmacological inhibition or by deprivation of oxygen in cells, causes cellular stress and growth arrest.

The most common physiological impediment to respiration is hypoxia. Respiration impairment can also be induced by genetic lesions of the ETC (e.g., in mitochondrial disorders) and by pharmaceuticals. Metformin, the first-line treatment for type 2 diabetes, acts at least in part as a complex I inhibitor. The consequences of ETC inhibition may vary depending on the respiratory chain component involved. For example, the reduction of complexes III and IV is increased by hypoxia but not by complex I inhibition.

It is generally considered that mitochondrial ATP generation from ETC can be compensated by maximizing glycolytic fluxes. The accumulation of mitochondrial NADH, however, could not be ameliorated via cytoplasmic glycolysis for no net NADH consumption. As a consequence of high NADH/NAD+ inside of mitochondria, cells are deprived the capacity to catabolize carbohydrate, amino acids, and fatty acids through TCA cycling. On this scenario, isocitrate dehydrogenase 1/2 (IDH1/2) prefer to reduce α-ketoglutarate into citrate for a depletion of intracellular citrate level. Glutamine reductive carboxylation becomes the primary acetyl-CoA source for cells in hypoxia or with defective mitochondria. Resulting from a stop of glutamine oxidation in mitochondria, cells are confronted with low abundance of cellular aspartate, whose de novo synthesis is proven to be the essential function of intact ETC. Low aspartate level limits protein and nucleotide synthesis, which can be rescued by overexpression of aspartate transporter SLC1A3 together with aspartate supplement. The accumulation of mitochondrial NADH, instead of a deficiency of ATP production in respiration deficient/redox imbalanced conditions, becomes toxic for cell growth.

It was unexpected that serine catabolism could be a major source of NADH and affect cellular redox state. In general, it is difficult to affect cellular redox state in vivo and there are many diseases for which doing so would be beneficial. The most straightforward are inborn errors of mitochondrial metabolism, which are catastrophic diseases with few treatment options. But redox status is involved in a great number of diseases. Metformin, a widely used drug, is a respiration inhibitor (i.e., NADH inducer). Pathological elevations in NADH may be key contributors to tissue damage by heart attack and stroke (as well as other ischemic events like renal ischemia).

Example causes of NADH elevation include, but are not limited to, mitochondrial electron transport chain deficiency (e.g., inborn error of metabolism, or due to aging or disease); hypoxia/anoxia (e.g., caused by heart attack, stroke, or other ischemic events, or associated with tumor microenvironments), impaired oxygenation due to excessive fat (e.g., in excess of adipose or fatty liver disease); and drug therapy (e.g., metformin treatment or treatment with other electron transport chain inhibitors, like phenformin, rotenone, and antimycin, or treatment with drugs having a side effect of ETC impairment).

A scientific foundation of this disclosure is the observation that serine catabolism becomes a major NADH source when respiration is impaired. This presents the possibility to manipulate NADH for therapeutic purposes via manipulating the serine pathway, for example, through substrate feeding, acceleration, or blockade. One outcome of doing so is improved fitness of animals with genetic deficiencies of the serine pathway. Excess NADH can also be a problem in ischemic diseases like heart attack, stroke, etc. Enhanced NADH likely plays a role in other diseases as well.

In other diseases (e.g., cancer) it can be advantageous to enhance NADH by activating the serine pathway, e.g., to help kill ischemic tumors.

In one aspect, the disclosure provides methods of treating disorders associated with excess NADH, such as, e.g., respiration impairment, in a subject (e.g., a subject in need thereof), comprising modulating serine metabolism (e.g., modulating serine catabolism) in the subject. In some embodiments, modulating comprises inhibiting serine metabolism (e.g., serine catabolism). In some embodiments, modulating serine metabolism comprises administering (e.g., a therapeutically effective amount of) a modulator (e.g., inhibitor) of serine metabolism (e.g., serine catabolism) to the subject.

In one aspect, the disclosure provides methods of treating disorders associated with excess NADH, such as, e.g., respiration impairment, in a subject (e.g., a subject in need thereof), comprising administering a composition (e.g., a therapeutically effective amount of a composition) to the subject that modulates (e.g., inhibits) serine catabolism.

In some embodiments, the composition comprises an antifolate. In some embodiments, the composition comprises a compound that inhibits SHMT2, MTHFD2 or a combination thereof. In one embodiment, the compound is KDG112.

In one aspect, the disclosure provides methods of treating a disorder associated with excess NADH, such as, e.g., respiration impairment, in a subject (e.g., a subject in need thereof), comprising administering an inhibitor of serine metabolism (e.g., a therapeutically effective amount of an inhibitor of serine metabolism) to the subject.

In some embodiments, the disorder is respiration impairment. In some embodiments, the respiration impairment is caused by a genetic deficiency of the electron transport chain. In another embodiment, the respiration impairment is caused by Leigh syndrome.

In some embodiments, the disorder is caused by hypoxia. In some embodiments, the disorder is caused by an ischemia. In some embodiments, the disorder is caused by a tumor. In some embodiments, the disorder is caused by impaired oxygenation due to excess adipose tissue. In some embodiments, the disorder is caused by an ischemic event.

In embodiments of any aspect disclosed herein, the inhibitor of serine metabolism is an inhibitor of serine catabolism.

In some embodiments, the inhibitor of serine catabolism comprises an antifolate. In some embodiments, the inhibitor of serine catabolism comprises or is a compound that inhibits SHMT (e.g., SHMT1 and/or SHMT2). In some embodiments, the inhibitor of serine catabolism comprises or is a compound the inhibits MTHFD2. In some embodiments, the inhibitor of serine catabolism comprises a compound that inhibits SHMT2, MTHFD2 or a combination thereof. In one embodiment, the inhibitor of serine catabolism is KDG112.

In some embodiments, the subject is receiving an electron transport chain inhibitor in combination with the inhibitor of serine metabolism (e.g., serine catabolism).

In some embodiments, the subject has received an electron transport chain inhibitor, such as metformin, phenformin, rotenone or antimycin.

In another aspect, the disclosure provides methods of treating a disorder associated with excess NADH, such as, e.g., respiration impairment, in a subject comprising modulating serine metabolism (e.g., serine catabolism) in the subject. In some embodiments, the respiration impairment is caused by a genetic deficiency of the electron transport chain. In further embodiments, the respiration impairment is caused by Leigh Syndrome.

In some embodiments, the modulating comprises inhibition of serine metabolism. In some embodiments, the modulating comprises inhibition of serine catabolism. In one embodiment, the modulating comprises disrupting the mitochondrial folate system.

In some embodiments, the respiration impairment is caused by hypoxia. In some embodiments, the disorder is caused by an ischemia. In some embodiments, the disorder is caused by a tumor. In some embodiments, the disorder is caused by impaired oxygenation due to excess adipose tissue. In some embodiments, the disorder is caused by an ischemic event.

In some embodiments, the NADH build-up, or excess, in the subject is caused by (or is a side effect of) electron transport chain inhibitors, such as metformin, phenformin, rotenone and/or antimycin.

In a further aspect, the disclosure provides methods of treating a mitochondrial disorder in a subject (e.g., a subject in need thereof), comprising administering an effective amount of a modulator of serine metabolism (e.g., an inhibitor of serine metabolism) to the subject, wherein the modulator of serine metabolism modulates mitochondrial serine metabolism. In some embodiments, the modulator of serine metabolism is a modulator of serine catabolism. In some embodiments, the modulator of serine catabolism is an inhibitor of serine catabolism.

In a further aspect, the disclosure provides methods of treating a mitochondrial disorder in a subject (e.g., a subject in need thereof), comprising administering a composition that modulates (e.g., inhibits) the mitochondrial serine catabolic pathway. In some embodiments, the composition comprises a compound that inhibits SHMT2, MTHFD2 or a combination thereof. In some embodiments, the composition comprises the compound KDG112.

In another aspect, the disclosure provides methods for treating a disease involving deficient respiration in a subject (e.g., a subject in need thereof), comprising administering a therapeutically effective amount of an inhibitor of serine metabolism (e.g., serine catabolism) to the subject.

In some embodiments, the disease is a genetically inherited mitochondrial disorder. In some embodiments, the disease is myocardial infarction or stroke.

In some embodiments of the various aspects described, the inhibitor of serine metabolism (e.g., serine catabolism) is an antifolate. In some embodiments of the various aspects described, the antifolate is a DIFR inhibitor, mitochondrial folate transporter inhibitor, MTHFD2L or MTHFD1L inhibitor or a combination thereof. In some embodiments of the various aspects described, the antifolate is methotrexate, pemetrexed, proguanil, pyrimethamine, lometrexol, AG2034, nolatrexed, raltitrexed, talotrexin, plevitrexed, edatrexate, pralatrexate, trimetrexate, piritrexim or trimethoprim, or a combination thereof.

In some embodiments of the various aspects described, the inhibitor of serine metabolism (e.g., serine catabolism) inhibits SHMT (e.g., SHMT1, SHMT2 or SHMT1 and SHMT2)

In some embodiments of the various aspects described, the inhibitor of serine metabolism (e.g., serine catabolism) inhibits SHMT2 or MTHFD2. In some embodiments of the various aspects described, the inhibitor of SHMT2 or MTHFD2 is a small molecule inhibitor. In some embodiments of the various aspects described, the inhibitor of SHMT2 is a compound described in U.S. Pat. No. 10,077,273B2, the contents of which are incorporated herein by reference. In some embodiments of the various aspects described, the inhibitor of SHMT2 is a compound described in Mainolfi. N., et al., Shmt inhibitors and uses thereof. PCT/US2017/064618, the contents of which are incorporated herein by reference.

In some embodiments of the various aspects described, the inhibitor of MTHFD2 is a compound described in PCT/US2016/045086, incorporated herein by reference. In some embodiments of the various aspects described, the inhibitor of MTHFD2 is a substrate-based inhibitor, such as but not limited to, LY345899. In some embodiments of the various aspects described, the inhibitor of MTHFD2 is carolacton, as described in Fu. C., et al. The natural product carolacton inhibits folate dependent C1 metabolism by targeting FolD/MTHFD. Nature Comm. 8, Article No. 1529, 2017, the contents of which are incorporated herein by reference.

In some embodiments of the various aspects described, the inhibitor of MTHFD2 is a substrate-based inhibitor that binds to the THE pocket of MTHFD2. In some embodiments of the various aspects described, the inhibitor of MTHFD2 is a substrate-based inhibitor that binds to the NAD pocket of MTHFD2. In certain embodiments, the MTHFD1L inhibitor is an inhibitor described in Asai, A., et al., Drug discovery of anticancer drugs targeting methylenetetrahydrofolate dehydrogenase 2. Heliyon 4 17 Dec. 2018—Volume 4, Issue 12, e01021, the contents of which are incorporated herein by reference. In some embodiments of the various aspects described, the MTHFD2 inhibitor is a compound as described in Ota, M., et al., Sulfonamide derivative having coumarin skeleton. PCT/JP2017/04194, the contents of which are incorporated herein by reference. In some embodiments of the various aspects described, the MTHFD2 inhibitor is a compound as described in Mainolfi N., et al., Caffeine inhibitors of MTHFD2 and uses thereof. PCT/US2016/066666, the contents of which are incorporated herein by reference. In some embodiments of the various aspects described, the MTHFD2 inhibitor is a compound as described in Eadsforth, T. C., et al., Characterization of 2, 4-Diamino-6-oxo-1,6- dihydropyrimidin-5-yl Ureido Based Inhibitors of Trypanosoma brucei FolD and Testing for Antiparasitic Activity. J Med Chem. 2015 Oct. 22; 58(20):7938-48. In some embodiments, the MTHFD2 inhibitor is a compound, LY374571, as described in Eadsforth, T. C., et al., Acinetobacter baumannii FolD ligand complexes—potent inhibitors of folate metabolism and a re-evaluation of the structure of LY374571. FEBS J. 2012 December; 279(23):4350-60, the contents of which are incorporated herein by reference.

In some embodiments of the various aspects described, the inhibitor of serine metabolism is an inhibitor of serine synthesis including but not limited to phosphoglycerate dehydrogenase (PHGDH), phosphoserine aminotransferase 1 (PSAT1) or phosphoserine phosphatase (PSPH). In some embodiments of the various aspects described, the inhibitor of serine metabolism is an inhibitor of serine import to mitochondria, e.g., via SFXN1. In some embodiments, the inhibitor of PHGDH is a compound described in Pacold, M. E., et al., A PHGDH inhibitor reveals coordination of serine synthesis and 1-carbon unit fate. Nat Chem Biol. 2016 June: 12(6): 452-458. In some embodiments of the various aspects described, the inhibitor of PHGDH is a piperazine-1-thiourea-based inhibitor of PHGDH, as described in Rhode, J. M., et al. Discovery and optimization of piperazine-1-thiourea-based human phosphoglycerate dehydrogenase inhibitors. Bioorganic & Medicinal Chemistry doi: 10.1016/j.bmc.2018.02.016, the teachings of which are incorporated herein by reference. In some embodiments of the various aspects described, the inhibitor of PHGDH is AstraZeneca PHGDH inhibitor, CBR 5884, Raze inhibitor, α-ketothioamide inhibitor, PKUMDL-WQ-2101, PKUMDL-WQ-2201, NCT-502 or NCT-503, as described in Ravez, S., et al., Challenges and Opportunities in the Development of Serine Synthetic Pathway Inhibitors for Cancer Therapy. J Med Chem 2017, 60 (4), 1227-1237; Fuller, N. O., et al., In Fragment-based discovery of the first known inhibitors of PHGDH, American Chemical Society: 2015; pp MEDI-366; Fuller, N. O., et al., An improved model for fragment-based lead generation at AstraZeneca. Drug Discov Today 2016, 21 (8), 1272-83; Mullarky, E., et al., Identification of a small molecule inhibitor of 3-phosphoglycerate dehydrogenase to target serine biosynthesis in cancers. Proc Natl Acad Sci USA 2016, 113 (7), 1778-83; Mullarky, E., et al., A novel small-molecule inhibitor of 3-phosphoglycerate dehydrogenase. Mol Cell Oncol 2016, 3 (4), e1164280; Pacold, M. E., et al., A PHGDH inhibitor reveals coordination of serine synthesis and one-carbon unit fate. Nat Chem Biol 2016, 12 (6), 452-8; Saiah, E. et al., 3-Phosphoglycerate dehydrogenase inhibitors as Antitumor agents. PCT2015/049149; Wang, Q., et al., Rational Design of Selective Allosteric Inhibitors of PHGDH and Serine Synthesis with Anti-tumor Activity. Cell Chem Biol 2017, 24 (1), 55-65; and Ravez, S., et al., alpha-Ketothioamide Derivatives: A Promising Tool to Interrogate. Phosphoglycerate Dehydrogenase (PHGDH). J Med Chem 2017, 60 (4), 1591-1597, the contents of which are incorporated herein by reference. In some embodiments of the various aspects described, the inhibitor of serine metabolism is an inhibitor targeting SHMT2 downstream enzymes, in one-carbon metabolism including but not limited to 5-FU and gemcitabine.

A modulator described herein (e.g., an inhibitor of serine catabolism) can be used in combination with one or more other therapies (e.g., one or more other therapeutically active agents, radiation therapy). When an agent is “in combination” with one or more other therapies, the agent and the one or more other therapies can be administered to a subject sequentially (in any order) or concurrently, or any combination thereof. When two or more agents are administered concurrently, the agents can be in separate formulations or the same formulation. Alternatively, the agents can be administered sequentially, as separate formulations. When an agent is “in combination” with one or more other therapies and the agent and the one or more other therapies are administered to a subject sequentially, the agent and the one or more other therapies should typically be administered to a subject within an appropriate time frame as determined by a skilled clinician (e.g., a time sufficient to allow an overlap of the pharmaceutical effects of the therapies).

In some embodiments, a method described herein further comprises administering to the subject a therapeutically effective amount of an additional therapy (e.g., an additional therapeutic agent, such as an electron transport chain inhibitor). In some embodiments, the additional therapy is administered in combination with the original agent or therapy.

In some embodiments of any aspect described herein, the methods further comprise administering (e.g., a therapeutically effective amount of) an electron transport chain inhibitor (e.g., metformin, IACS-10759) to the subject, e.g. in combination with a modulator of serine metabolism (e.g., an inhibitor of serine metabolism, an inhibitor of serine catabolism) or serine supplement. In some embodiments, the electron transport chain inhibitor and the modulator of serine metabolism are administered sequentially. In some embodiments, the electron transport chain inhibitor and the modulator of serine metabolism are administered concurrently, e.g., in the same formulation or in separate formulations.

In some embodiments of the various aspects described, the disclosure provides methods of treating a mitochondrial disease by administering to the subject in need thereof compounds that modulate folate-mediated NADH generation separate from serine, including but not limited to, compounds that modulate catabolism of glycine, choline, trimethylglycine (betaine), dimethylglycine, and methylglycine (sarcosine). In some embodiments, the compounds that modulate folate-mediated NADH generation separate from serine include but are not limited to modulators of dimethylglycine dehydrogenase, sarcosine dehydrogenase or ALDH7A1. In some embodiments, the disclosure provides methods of treating a mitochondrial disease wherein the inhibitor of serine metabolism is administered in combination with one or more anti-cancer drugs. In certain embodiments, the disclosure provides methods wherein the inhibitor of serine metabolism is administered in combination with an anti-cancer drug or anti-cancer therapy that causes mitochondrial dysfunction. In some embodiments, the anti-cancer drugs include but are not limited to monoclonal antibodies against tyrosine kinase receptors, tyrosine kinase inhibitors, and antiangiogenic drugs. In some embodiments, the anti-cancer drugs include drugs related to but not limited to doxorubicin, paclitaxel, cyclophosphamide, ifosfamide, melphalan, chlorambucil, dacarbazine, cisplatin, oxaliplatin, idarubicin, mitoxantrone, etoposide, dexamethasone, tamoxifen, anti-aromatase, anti-androgens, anti-estrogens, LH-RH agonists, retinoids or a combination thereof. In some embodiments, the anti-cancer drug is the compound, deguelin, as described in Wang. Y., et al., Deguelin, a novel anti- tumorigenic agent targeting apoptosis, cell cycle arrest and anti-angiogenesis for cancer chemoprevention. Mol Clin Oncol. 2013 March-April; 1(2): 215-219, the teachings of which are incorporated herein by reference. In some embodiments, the anti-cancer drugs include drugs related to but not limited to doxorubicin, trastazumab or sunitinib or a combination thereof. In some embodiments, the disclosure provides methods wherein the inhibitor of serine metabolism is administered in combination with radiation therapy.

In another aspect, the disclosure provides methods for treating a mitochondrial disease in a subject (e.g., a subject in need thereof), comprising administering a diet or dietary supplement deficient in serine compared to other amino acids to the subject.

In another aspect, the disclosure provides methods of improving the therapeutic index of an electron transport chain inhibitor (e.g., metformin, IACS-10759) in a subject, comprising administering a therapeutically effective amount of an inhibitor of serine metabolism (e.g., an inhibitor of serine catabolism) to the subject, wherein the subject is receiving the electron transport chain inhibitor in combination with the inhibitor of serine metabolism. In some embodiments, the methods further comprise administering (e.g., a therapeutically effective amount of) the electron transport chain inhibitor (e.g., metformin, IACS-10759) to the subject.

In another aspect, the disclosure provides methods of improving the therapeutic index of metformin in a subject (e.g., a subject in need thereof), comprising administering a therapeutically effective amount of an inhibitor of serine metabolism to the subject.

The therapeutic index of a drug is the ratio of the dose of a drug that achieves a predetermined toxicity endpoint (e.g., toxic dose in 50% of subjects, TD₅₀) to the dose of drug that achieves a desired pharmacological effect (e.g., efficacious dose in 50% of subjects, ED₅₀). A skilled worker can calculate the therapeutic index of a drug.

In a further aspect, the disclosure provides methods for improving the effectiveness of an electron transport chain inhibitor (e.g., metformin, IACS-10759) in a subject, comprising administering to the subject an effective amount of a serine supplement, wherein the subject is receiving the electron transport chain inhibitor in combination with the serine supplement. In some embodiments, the methods further comprise administering (e.g., a therapeutically effective amount of) the electron transport chain inhibitor (e.g., metformin, IACS-10759) to the subject.

In a further aspect, the disclosure provides methods for improving the effectiveness of metformin in a subject (e.g., a subject in need thereof), comprising administering a serine supplement to the subject.

Examples of electron transport chain inhibitors include metformin, phenformin, rotenone, deguelin, antimycin and oligomycin. A further example of an electron transport chain inhibitor is IACS-10759. In some embodiments, an electron transport chain inhibitor is metformin, phenformin, rotenone, deguelin, antimycin, oligomycin or a combination thereof. In some embodiments, an electron transport chain inhibitor is metformin. In some embodiments, an electron transport chain inhibitor is IACS-10759.

Serine supplements can contain L-serine and/or D-serine, but typically contain L-serine. Choline or phosphatidylcholine can also be used to provide one-carbon unit supplementation and thereby mimic serine supplementation. The following foods are examples of sources of L-serine that can be used as a serine supplement: eggs, soy protein, fish, bacon and turkey.

In another aspect, the disclosure provides methods of treating a tumor in a subject (e.g., a subject in need thereof), comprising administering to the subject an effective amount of an activator of serine catabolism. In one embodiment, the method of treating a tumor further comprises administering an electron transport chain inhibitor to the subject. The electron transport chain inhibitor can be, e.g., metformin, phenformin, rotenone, deguelin, antimycin, oligomycin, or a combination thereof.

The administration of the agents, modulators (e.g., inhibitors) and compositions (e.g., compositions) described herein may be carried out in any manner, e.g., by parenteral or nonparenteral administration, including by aerosol inhalation, injection, infusions, ingestion, implantation or transplantation. For example, the agents, modulators and compositions (e.g., compositions) described herein may be administered to a patient trans-arterially, intradermally, subcutaneously, intratumorally, intramedullary, intranodally, intramuscularly, by intravenous (i.v.) injection, or intraperitoneally. For example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration may all be used, depending, for example, on the agent, inhibitor or composition and the particular disease to be treated. Administration can be local or systemic as indicated. In one aspect, the agent, inhibitor or composition (e.g., composition) is administered by i.v. injection. In one aspect, the agent, inhibitor or composition (e.g., composition) is administered to a subject by intradermal or subcutaneous injection.

Delivery systems useful in the context of embodiments of the disclosure may include time-released, delayed release, and sustained release delivery systems such that the delivery of the drugs/compounds occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the disclosure.

In some embodiments, the methods and compositions described herein, and in some of each and every embodiment, the modulators of serine metabolism (e.g., modulator of serine catabolism, inhibitor of serine catabolism) may be provided in suitable pharmaceutical compositions comprising the modulator of serine metabolism and a pharmaceutically acceptable carrier. The carrier may be diluent, adjuvant, excipient, or vehicle with which the modulator (e.g., inhibitor) of serine metabolism is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or antibodies of the disclosure in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 2P1 Edition, Troy, D. B. ed., Lipincott\Williams and Wilkins, Philadelphia, Pa. 2006, Part 5, Pharmaceutical Manufacturing pp 691-1092, see especially pp. 958-989.

It should also be understood that, unless clearly indicated to the contrary, in any methods described herein that include more than one step or act, the order of the steps or acts of the method is not necessarily limited to the order in which the steps or acts of the method are recited.

### SHMT Inhibitor Compounds

Inhibitors (e.g., small molecule inhibitors) of SHMT, such as inhibitors of SHMT1 and/or SHMT2, and methods of making and using the same, are described in U.S. Patent Application Publication Nos. US 2018/0117010 and US 2018/0072751, the contents of which are incorporated herein by reference in their entireties. SHMT inhibitors (e.g., SHMT1 and/or SHMT2 inhibitors), including any of the SHMT inhibitors described herein, can be used in the methods described herein.

Accordingly, in some embodiments, a modulator of serine metabolism described herein (e.g., a modulator of serine catabolism, such as an inhibitor of serine catabolism) is a SHMT inhibitor. In some embodiments, the SHMT inhibitor is a SHMT1 inhibitor. In some embodiments, the SHMT inhibitor is a SHMT2 inhibitor. In some embodiments, the SHMT inhibitor is a SHMT1 inhibitor and a SHMT2 inhibitor.

An embodiment of an SHMT inhibitor is a compound of Formula (I):

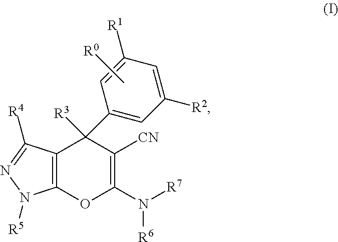

or a pharmaceutically acceptable salt thereof, wherein:
R⁰, R¹ and R² are each independently selected from the group consisting of —H, halogen (such as F, Br, or Cl), hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl (such as C₂-C₈ alkenyl), substituted or unsubstituted alkynyl (such as C₂-C₈ alkynyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy); provided that, at least one of R⁰, R¹ and R² is selected from the group consisting of substituted or unsubstituted alkenyl (such as C₂-C₈ alkenyl), and substituted or unsubstituted alkynyl (such as C₂-C₈ alkynyl);
R³ is selected from the group consisting of —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy);
R⁴ is selected from the group consisting of —H, substituted or unsubstituted alkyl (such as C₁-C₈alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl;
R⁵, R⁶ and R⁷ are each independently selected from the group consisting of —H, —C(O)R¹¹, substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹ is independently selected from the group consisting of substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl.

In some embodiments, an SHMT inhibitor is represented by Formula (Ia):

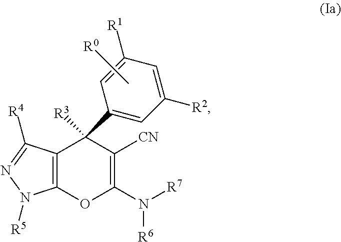

or a pharmaceutically acceptable salt there.

In certain embodiments, an SHMT inhibitor is represented by Formula (Ib):

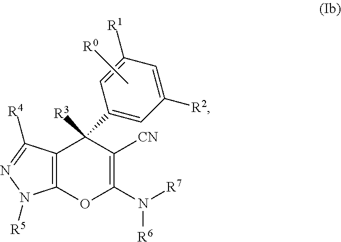

or a pharmaceutically acceptable salt thereof.

In certain embodiments of any of the foregoing or following, R⁰, R¹ and R² are each independently selected from the group consisting of —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl (such as C₂-C₈ alkenyl), substituted or unsubstituted alkynyl (such as C₂-C₈ alkynyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy); provided that, at least one of R⁰, R¹ and R² is independently selected from the group consisting of substituted or unsubstituted alkenyl, and substituted or unsubstituted alkynyl.

In certain embodiments of any of the foregoing or following, one of R¹ and R² is selected from the group consisting of substituted or unsubstituted alkenyl (such as C₂-C₈ alkenyl), and substituted or unsubstituted alkynyl (such as C₂-C₈ alkynyl); the other is independently selected from the group consisting of —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl (such as C₂-C₈ alkenyl), substituted or unsubstituted alkynyl (such as C₂-C₈ alkynyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy).

In other embodiments of any of the foregoing or following, one of R¹ and R² is selected from the group consisting of substituted or unsubstituted alkenyl (such as C₂-C₈ alkenyl), and substituted or unsubstituted alkynyl (such as C₂-C₈ alkynyl); the other is independently selected from the group consisting of —H, halogen, hydroxyl, nitro, nitrile, —OR¹¹, substituted or unsubstituted alkyl, substituted or unsubstituted alkenyl, substituted or unsubstituted alkynyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈haloalkoxy or C₁-C₆ haloalkoxy).

In certain embodiments of any of the foregoing or following, one of R¹ and R² is substituted or unsubstituted alkynyl (such as C₂-C₈ alkynyl), the other is not. In other embodiments, both R¹ and R² are substituted or unsubstituted alkynyl (such as C₂-C₈ alkynyl).

In some embodiments of any of the foregoing or following, one of R¹ and R² is substituted or unsubstituted alkenyl (such as C₂-C₈ alkenyl), the other is not. In other embodiments, both R¹ and R² are substituted or unsubstituted alkenyl (such as C₂-C₈ alkenyl).

In certain embodiments of any of the foregoing or following, R¹ is substituted or unsubstituted alkynyl and R² is not a substituted or unsubstituted alkynyl (such as C₂-C₈ alkynyl). In other embodiments, R² is substituted or unsubstituted alkynyl and R¹ is not a substituted or unsubstituted alkynyl (such as C₂-C₈ alkynyl).

In certain embodiments of any of the foregoing or following, R¹ is substituted or unsubstituted alkenyl and R² is not a substituted or unsubstituted alkenyl (such as C₂-C₈ alkenyl). In other embodiments, R² is substituted or unsubstituted alkenyl and R¹ is not a substituted or unsubstituted alkenyl (such as C₂-C₈ alkenyl).

In certain embodiments of any of the foregoing or following, the alkenyl (such as C₂-C₈ alkenyl) or alkynyl (such as C₂-C₈ alkynyl), when substituted, is substituted with one or more substituents independently selected from the group consisting of halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², optionally substituted aryl, and optionally substituted heteroaryl comprising 1-4 N atoms; or two of the substituents together with the atoms to which they are attached form an optionally substituted ring.

In certain such embodiments of any of the foregoing or following, alkenyl (such as C₂-C₈ alkenyl) or alkynyl (such as C₂-C₈ alkynyl), when substituted, is substituted with one or more substituents independently selected from the group consisting of OH, halogen, —OR¹¹, —C(O)OR¹², —C(O)NR¹⁰R¹², —NR¹⁰R¹², optionally substituted aryl, and optionally substituted heteroaryl comprising 1-4 N atoms; or two of the substituents together with the atoms to which they are attached form an optionally substituted ring.

In certain embodiments of any of the foregoing or following, R⁰ is selected from the group consisting of hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, and —NS(O)₂R¹². In certain such embodiments, R⁰ is H.

In certain embodiments of any of the foregoing or following, R³ is selected from the group consisting of methyl, ethyl, propyl, isopropyl, cyclopropyl, and cyclobutyl. In certain such embodiments, R³ is selected from the group consisting of isopropyl, cyclopropyl, and cyclobutyl. In some embodiments, R³ is isopropyl.

In certain embodiments of any of the foregoing or following, R⁴ is selected from the group consisting of methyl, ethyl, isopropyl, cyclopropyl, cyclobutyl, cyclopentyl, cyclohexyl, phenyl, and benzyl. In certain such embodiments, R⁴ is methyl or isopropyl. In some embodiments, R⁴ is methyl.

In certain embodiments of any of the foregoing or following, R⁵, R⁶, and R⁷ are each independently selected from the group consisting of —H, methyl, ethyl, isopropyl, cyclopropyl, cyclobutyl, cyclopentyl, cyclohexyl, phenyl, benzyl, and —COCH₃. In certain such embodiments, R⁵, R⁶ and R⁷ are each independently selected from the group consisting of —H, methyl, phenyl, and —COCH₃. In some embodiments, R⁵ and R⁶ are each independently selected from the group consisting of —H, methyl, and phenyl.

In certain embodiments of any of the foregoing or following, R⁷ is —H.

In certain embodiments of any of the foregoing or following, R⁵ is selected from the group consisting of —H, methyl, ethyl, isopropyl, cyclopropyl, cyclobutyl, cyclopentyl, cyclohexyl, phenyl, benzyl, and —COCH₃; and R⁶ and R⁷ taken together with the nitrogen atom to which they are attached form a substituted or unsubstituted ring selected from the group consisting of:

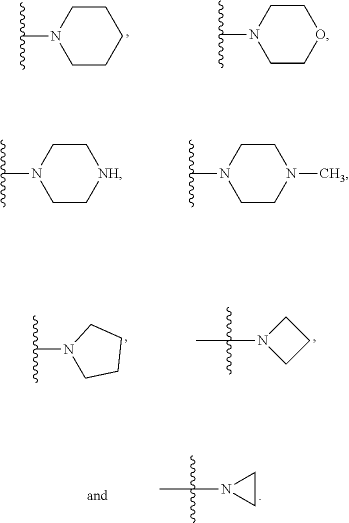

In certain embodiments of any of the foregoing or following, R⁵ is selected from the group consisting of —H, methyl, phenyl, and —COCH₃; and R⁶ and R⁷ taken together with the nitrogen atom to which they are attached form a substituted or unsubstituted ring selected from the group consisting of;

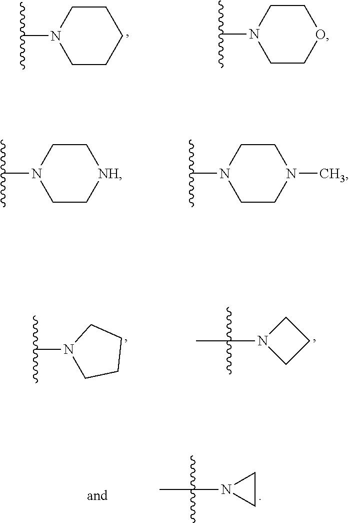

In certain embodiments of any of the foregoing or following, R⁰ is selected from the group consisting of —H, halogen, hydroxyl, nitro, nitrile, —S(O)₂R¹¹, —S(O)₂NR¹⁰R¹², —OR¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy);

one of R¹ and R² is substituted or unsubstituted alkenyl (such as C₂-C₈ alkenyl), or substituted or unsubstituted alkynyl (such as C₂-C₈ alkynyl); the other is independently selected from the group consisting of —H, halogen, hydroxyl, nitro, nitrile, —S(O)₂R¹¹, —S(O)₂NR¹⁰R¹², —OR¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl; e.g., methyl, ethyl, or iso-propyl), unsubstituted or unsubstituted alkenyl (such as C₂-C₈ alkenyl), substituted or unsubstituted alkynyl (such as C₂-C₈ alkynyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl); e.g., trifluoromethyl), and substituted or unsubstituted haloalkoxy (such as C₁-C₈ haloalkoxy or C₁-C₆ haloalkoxy);

R³ is selected from the group consisting of methyl, ethyl, propyl, isopropyl, cyclopropyl, and cyclobutyl;

R⁴ is selected from the group consisting of methyl, ethyl, isopropyl, cyclopropyl, cyclobutyl, cyclopentyl, cyclohexyl, phenyl and benzyl; and

R⁵, R⁶ and R⁷ are each independently selected from the group consisting of —H, methyl, ethyl, isopropyl, cyclopropyl, cyclobutyl, cyclopentyl, cyclohexyl, phenyl, benzyl, and —COCH₃.

In certain embodiments of any of the foregoing of following, R⁰ is —H;

one of R¹ and R² is substituted or unsubstituted alkenyl (such as C₂-C₈ alkenyl), or substituted or unsubstituted alkynyl (such as C₂-C₈ alkenyl); the other is independently selected from the group consisting of —H, halogen, hydroxyl, nitro, nitrile, —OR¹¹, substituted or unsubstituted alkyl (such as C₁-C₈ alkyl C₁-C₆ alkyl); e.g., methyl, ethyl, or iso-propyl), substituted or unsubstituted alkenyl (such as C₂-C₈ alkenyl), substituted or unsubstituted alkynyl (such as C₂-C₈ alkynyl), substituted or unsubstituted haloalkyl (such as C₁-C₈ haloalkyl or C₁-C₆ haloalkyl); e.g., trifluoromethyl), or substituted or unsubstituted haloalkoxy (such as C₁-C₈haloalkoxy or C₁-C₆ haloalkoxy);

R³ is selected from the group consisting of isopropyl, cyclopropyl, and cyclobutyl;

R⁴ is methyl or isopropyl; and

R⁵, R⁶ and R⁷ are each independently selected from the group consisting of —H, alkyl (such as C₁-C₈ alkyl C₁-C₆ alkyl); e.g., methyl, ethyl, or iso-propyl), phenyl, and —COCH₃.

In certain embodiments of any of the following or foregoing, R⁰ is —H;

one of R¹ and R² is substituted or unsubstituted alkenyl (such as C₂-C₈ alkenyl), or substituted or unsubstituted alkynyl (such as C₂-C₈ alkynyl); the other is independently selected from the group consisting of —H, methoxy, fluoro, chloro, bromo, hydroxyl, nitro, nitrile, alkyl, —CCl₃, and —CF₃;

R³ is cyclobutyl or iso-propyl;

R⁴ is methyl;

R⁵ and R⁶ are each independently selected from the group consisting of —H, alkyl (such as C₁-C₆alkyl; e.g., methyl, ethyl, or iso-propyl), and phenyl; and

R⁷ is H.

In certain embodiments of any of the foregoing or following, R⁰ is selected from the group consisting of —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, and —NS(O)₂R¹²;

one of R¹ and R² is substituted or unsubstituted alkynyl (such as C₂-C₈ alkynyl); the other is nitro, —Cl, —OCH₃, or —CF₃; R³ is iso-propyl; R⁴ is methyl; and R⁵, R⁶, and R⁷ are H.

In certain embodiments, an SHMT inhibitor is a compound of Formula (II):

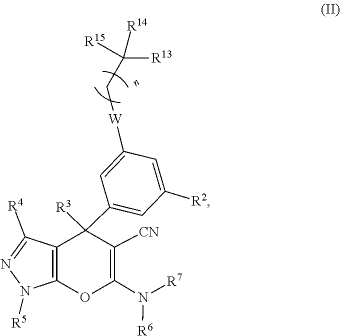

or a pharmaceutically acceptable salt thereof, wherein

R², R³, R⁴, R⁵, R⁶, and R⁷ are as defined herein;

W represents —CR¹⁶═CR¹⁶— or —C≡C—;

n is 0, 1, 2, 3, or 4;

R¹³, R¹⁴, and R¹⁵ are independently selected from the group consisting of hydrogen, —OH, halogen, optionally substituted alkyl (such as C₁-C₈ alkyl or C₁-C₆ alkyl); e.g., methyl, ethyl, or iso-propyl), optionally substituted haloalkyl (such as C₁-C₈ haloalkyl or C₁-C₆ haloalkyl; e.g., trifluoromethyl), —OR^{a}, —OC(O)R^{b}, —C(O)NR^{a}R^{b}, and —NR^{a}R^{b}; or R¹³ and R¹⁴ together with the atom to which they are attached form a 4-7 membered heterocyclic ring comprising 1 or 2 heteroatoms selected from the group consisting of NR^{a}, O, S, or SO, or SO₂; wherein the heterocyclic ring is optionally substituted with one or more substituents independently selected from the group consisting of oxo and optionally substituted alkyl; and R¹⁶, R^{a} and R^{b}, independently at each occurrence, are H or optionally substituted alkyl (such as C₁-C₈ alkyl or C₁-C₆ alkyl; e.g., methyl, ethyl, or iso-propyl).

In certain embodiments, the compound of Formula (II) can be represented by Formula (IIa) or (IIb):

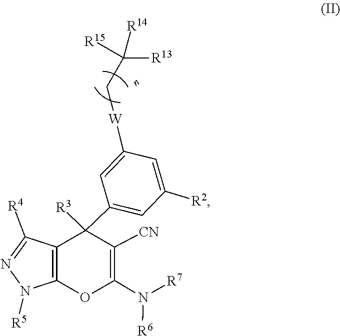

pharmaceutically acceptable salt thereof.

In certain embodiments of any of the foregoing or following, W is —CR¹⁶═CR¹⁶ in cis- or trans-formation. In other embodiments, W is —C≡C—.

In certain embodiments of any of the foregoing or following, R¹³, R¹⁴, and R¹⁵ are independently selected from the group consisting of hydrogen, —OH, halogen, optionally substituted alkyl (such as C₁-C₈ alkyl or C₁-C₆ alkyl; e.g., methyl, ethyl, or iso-propyl), optionally substituted haloalkyl (such as C₁-C₈ haloalkyl or C₁-C₆ haloalkyl; e.g., trifluoromethyl), —OR^{a}, —OC(O)R^{b}, —C(O)NR^{a}R^{b}, and —NR^{a}R^{b}, and wherein R^{a} and R^{b}, independently at each occurrence, are H or optionally substituted alkyl ((such as C₁-C₈ alkyl or C₁-C₆ alkyl; e.g., methyl, ethyl, or iso-propyl).

In certain embodiments of any of the foregoing or following, n is 0; R¹³ and R¹⁴ together with the atom to which they are attached form a 4-7 membered heterocyclic ring comprising 1 or 2 heteroatoms selected from the group consisting of NR^{a}, and O; wherein the heterocyclic ring is optionally substituted with one or more substituents independently selected from the group consisting of oxo and optionally substituted alkyl; and R¹⁵ is H. The disclosure contemplates compounds have any combination of any of the foregoing or following structural and/or functional characteristics.

In certain embodiments of any of the foregoing or following, the SHMT inhibitor is a compound of Formula (III):

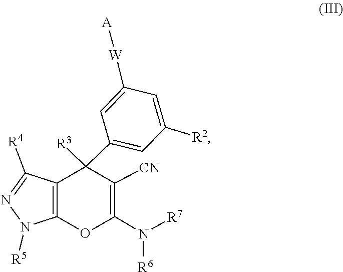

or a pharmaceutically acceptable salt thereof,

wherein

R², R³, R⁴, R⁵, R⁶, and R⁷ are as defined herein;

W represents —CR¹⁶═CR¹⁶ or —C≡C—;

R¹⁶ is H or optionally substituted alkyl (such as C₁-C₈ alkyl or C₁-C₆ alkyl; e.g., methyl, ethyl, or iso-propyl); and

A represents optionally substituted aryl or optionally substituted heteroaryl.

In certain embodiments, the compound of Formula (III) can be represented by Formula (IIIa) or (IIIb):

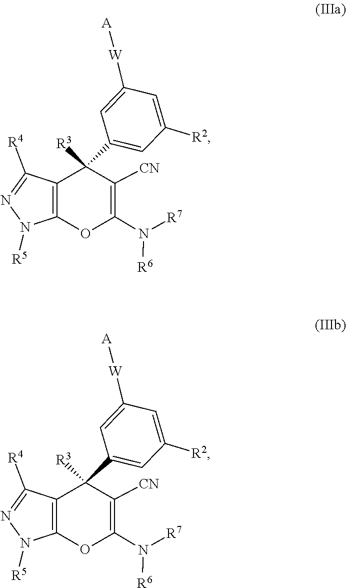

or a pharmaceutically acceptable salt thereof.

In certain embodiments of any of the foregoing or following, W is —C≡C—. In other embodiments, W is —CR¹⁶=CR¹⁶ in cis- or trans-formation.

In certain embodiments of any of the foregoing or following, A is aryl, optionally substituted with one or more substituents independently selected from the group consisting of —OH, halogen, optionally substituted alkyl (such as C₁-C₉ alkyl or C₁-C₆ alkyl; e.g., methyl, ethyl, or iso-propyl), optionally substituted haloalkyl (such as C₁-C₈ haloalkyl or C₁-C₆ haloalkyl; e.g., trifluoromethyl), —OR^{a}, —OC(O)R^{b}, —C(O)NR^{a}R^{b}, and —NR^{a}R^{b}, and wherein R^{a} and R^{b}, independently at each occurrence, are H or optionally substituted alkyl. In certain such embodiments, A is phenyl, optionally substituted with one or more substituents independently selected from the group consisting of —CH₂OH, —OH, —CF₃, —COOH, —F, —CH₂NH₂, —CONH₂, and —NH₂.

In certain embodiments of any of the foregoing or following, A is heteroaryl, optionally substituted with one or more substituents independently selected from the group consisting of —OH, halogen, optionally substituted alkyl (such as C₁-C₈ alkyl or C₁-C₆ alkyl; e.g., methyl, ethyl, or iso-propyl), optionally substituted haloalkyl (such as C₁-C₈ haloalkyl or C₁-C₆ haloalkyl; e.g., trifluoromethyl), —OR^{a}, —OC(O)R^{b}, —C(O)NR^{a}R^{b}, and —NR^{a}R^{b}, and wherein R^{a} and R^{b}, independently at each occurrence, are H or optionally substituted alkyl (such as C₁-C₈ alkyl or C₁-C₆ alkyl; e.g., methyl, ethyl, or iso-propyl).

In some embodiments of any of the foregoing or following, A is an optionally substituted heteroaryl containing 1-4 N atoms.

In some embodiments of any of the foregoing or following, A is an optionally substituted tetrazolyl or optionally substituted triazolyl.

In certain embodiments of any of the foregoing or following, A is pyridinyl, optionally substituted with one or more substituents independently selected from the group consisting of —H, —CH₂OH, —OH, —CF₃, —COOH, —F, —CH₂NH₂, —CONH₂, and —NH2.

In certain embodiments of any of the foregoing or following, R² is nitro, —F, —Cl, —OCH₃, CCl₃, or —CF₃; R³ is selected from the group consisting of isopropyl, cyclopropyl, and cyclobutyl; R⁴ is methyl or isopropyl; and R⁵, R⁶ and R⁷ are each independently selected from the group consisting of —H, alkyl (such as C₁-C₈ alkyl or C₁-C₆ alkyl; e.g., methyl, ethyl, or iso-propyl), phenyl, and —COCH₃.

In certain embodiments of any of the foregoing or following, R² is —CF₃; R³ is iso-propyl; R⁴ is methyl; and R⁵, R⁶, and R⁷ are H.

In certain embodiments, an SHMT inhibitor is a compound of Formula (II′):

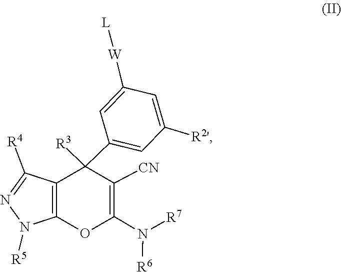

or a pharmaceutically acceptable salt thereof, wherein:
R² and R³ are each independently selected for each occurrence from the group consisting of —H, halogen (such as F, Br, or Cl), hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy);
R⁴ is selected from the group consisting of —H, substituted or unsubstituted alkyl (such as C₁-C₈alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl;
R⁵, R⁶ and R⁷ are each independently selected from the group consisting of —H, —C(O)R¹¹, substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹ is independently selected from the group consisting of substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl;
W represents —CR¹⁶═CR¹⁶— or —C≡C—;

R¹⁶ is H or optionally substituted alkyl (such as C₁-C₈ alkyl or C₁-C₆ alkyl; e.g., methyl, ethyl, or iso-propyl); and

L is selected from the group consisting of —H, substituted or unsubstituted alkyl (such as C₁-C₁₂ alkyl or C₁-C₈ alkyl; e.g., methyl, ethyl, iso-propyl, n-butyl, or n-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted arylalkyl, substituted or unsubstituted heteroarylalkyl.

In certain embodiments, the compound of Formula (II′) can be represented by Formula (IIa′) or (IIb′):

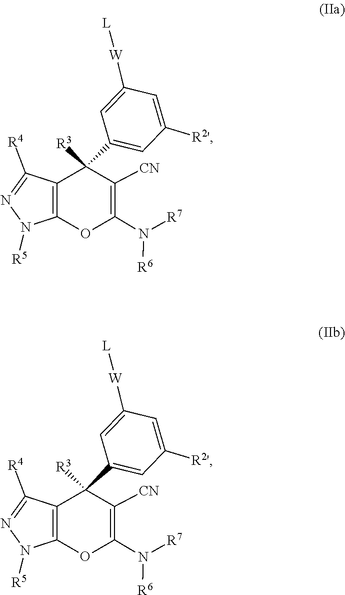

or a pharmaceutically acceptable salt thereof.

In certain embodiments of any of the foregoing or following, W is —CR¹⁶═CR¹⁶ in a cis-formation. In certain embodiments, W is —CR¹⁶═CR¹⁶ in a trans-formation. In some embodiments, W is —C≡C—.

In certain embodiments of any of the foregoing or following, L is alkyl, optionally substituted with one or more substituents selected from the group consisting of halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₁₂ alkyl or C₁-C₈ alkyl; e.g., methyl, ethyl, iso-propyl, or n-butyl), substituted or unsubstituted cycloalkyl (such as C₃-C₇ cycloalkyl; e.g., cyclopropyl or cyclobutyl), substituted or unsubstituted heterocyclyl ring, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy).

In certain embodiments of any of the foregoing or following, L is alkyl optionally substituted with one or more substituents independently selected from the group consisting of hydrogen, —OH, halogen, optionally substituted alkyl (such as C₁-C₁₂ alkyl or C₁-C₈ alkyl; e.g., methyl, ethyl, iso-propyl, n-butyl), substituted or unsubstituted heteroaryl, optionally substituted haloalkyl (such as C₁-C₈ haloalkyl or C₁-C₆ haloalkyl; e.g., trifluoromethyl), —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹².

In certain embodiments of any of the foregoing or following, L is cycloalkyl or heterocyclyl ring, optionally substituted with one or more substituents selected from the group consisting of halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl, and oxo.

In certain embodiments of any of the foregoing or following, L is a four to seven membered heterocyclyl ring comprising one to three heteroatoms selected from the group consisting of NR¹⁰, O, S, SO, or SO₂.

In certain embodiments of any of the foregoing or following, L is H.

In certain embodiments of any of the foregoing or following, an SHMT inhibitor is a compound of Formula (III′):

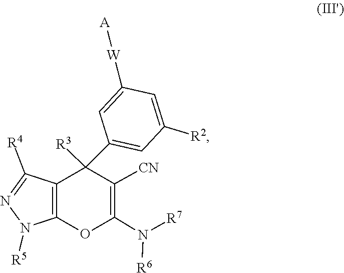

or a pharmaceutically acceptable salt thereof,

wherein:
R² and R³ are each independently selected for each occurrence from the group consisting of —H, halogen (such as F, Br, or Cl), hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy);
R⁴ is selected from the group consisting of —H, substituted or unsubstituted alkyl (such as C₁-C₈alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl;
R⁵, R⁶ and R⁷ are each independently selected from the group consisting of —H, —C(O)R¹¹, substituted or unsubstituted alkyl (such as C₁-C₈ alkyl or C₁-C₆ alkyl; e.g., methyl, ethyl, or iso-propyl), substituted or unsubstituted cycloalkyl (such as C₃-C₇ cycloalkyl; e.g., cyclopropyl or cyclobutyl), substituted or unsubstituted aryl, substituted or unsubstituted heteroaryl, substituted or unsubstituted arylalkyl, and substituted or unsubstituted heteroarylalkyl; or R⁵ is selected from any of the foregoing and R⁶ and R⁷ taken together with the nitrogen atom to which they are attached form a substituted or unsubstituted 3-6 membered ring;
each occurrence of R¹¹ is independently selected from the group consisting of substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl; and
each occurrence of R¹⁰ and R¹² is independently selected from the group consisting of —H, substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, and substituted or unsubstituted heteroaryl;
W represents —CR¹⁶═CR¹⁶ or —C≡C—;

R¹⁶ is H or optionally substituted alkyl (such as C₁-C₈ alkyl or C₁-C₆ alkyl; e.g., methyl, ethyl, or iso-propyl); and

A represents optionally substituted aryl or optionally substituted heteroaryl.

In certain embodiments, the compound of Formula (III) can be represented by Formula (IIIa′) or (IIIb′):

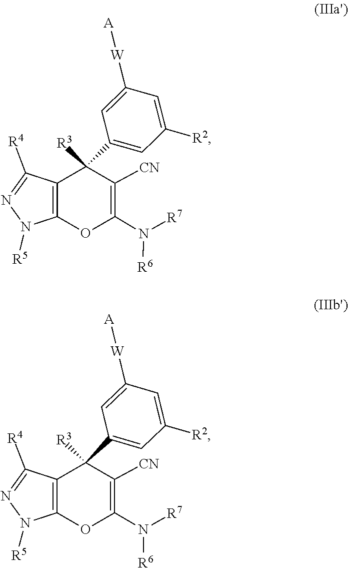

or a pharmaceutically acceptable salt thereof.

In certain embodiments of any of the foregoing or following, W is —C≡C—. In some embodiments, W is —CR¹⁶=CR¹⁶ in a cis-formation. In some embodiments, W is in a trans-formation.

In certain embodiments of any of the foregoing or following, A is aryl, optionally substituted with one or more substituents independently selected from the group consisting of halogen (such as F, Br, or Cl), hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy).

In certain such embodiments, A is phenyl, optionally substituted with one or more substituents independently selected from the group consisting of —CH₂OH, —OH, —CF₃, —COOH, —F, —CH₂NH₂, —CONH₂, and —NH₂.

In certain embodiments of any of the foregoing or following, A is heteroaryl, optionally substituted with one or more substituents independently selected from the group consisting of halogen (such as F, Br, or Cl), hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted alkyl (such as C₁-C₈ alkyl or C₁-C₆ alkyl; e.g., methyl, ethyl, or iso-propyl), substituted or unsubstituted alkenyl, substituted or unsubstituted alkynyl, substituted or unsubstituted cycloalkyl (such as C₃-C₇ cycloalkyl; e.g., cyclopropyl or cyclobutyl), substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl (such as C₁-C₈ haloalkyl or C₁-C₆ haloalkyl; e.g., trifluoromethyl), and substituted or unsubstituted haloalkoxy (such as C₁-C₈ haloalkoxy or C₁-C₆ haloalkoxy).

In certain embodiments of any of the foregoing or following, A is heteroaryl, optionally substituted with one or more substituents independently selected from the group consisting of —OH, halogen, optionally substituted alkyl (such as C₁-C₈ alkyl or C₁-C₆ alkyl; e.g., methyl, ethyl, or iso-propyl), optionally substituted haloalkyl (such as C₁-C₈ haloalkyl or C₁-C₆ haloalkyl; e.g., trifluoromethyl), —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹².

In certain such embodiments, A is an optionally substituted heteroaryl containing 1-4 N atoms.

In some embodiments of any of the foregoing or following, A is an optionally substituted tetrazolyl or optionally substituted triazolyl.

In certain embodiments of any of the foregoing or following, A is pyridinyl, optionally substituted with one or more substituents independently selected from the group consisting of —H, —CH₂OH, —OH, —CF₃, —COOH, —F, —CH₂NH₂, —CONH₂, and —NH2.

In certain embodiments of any of the foregoing or following, R² is nitro, —F, —Cl, —OCH₃, CCl₃, or —CF₃; R³ is selected from the group consisting of isopropyl, cyclopropyl, and cyclobutyl; R⁴ is methyl or isopropyl; and R⁵, R⁶ and R⁷ are each independently selected from the group consisting of —H, alkyl (such as C₁-C₈ alkyl or C₁-C₆ alkyl; e.g., methyl, ethyl, or iso-propyl), phenyl, and —COCH₃.

In certain embodiments of any of the foregoing or following, R² is —CF₃; R³ is iso-propyl; R⁴ is methyl; and R⁵, R⁶, and R⁷ are H.

In certain embodiments of any of the foregoing or following, the SHMT inhibitor is selected from a compound in Table 1, or a pharmaceutically acceptable salt thereof.

TABLE 1
1,4-dihydropyrano[2,3-c]pyrazole derivatives as SHMT inhibitors

| Compound | |
|---|---|
| # | Chemical Structure |
| 1 | 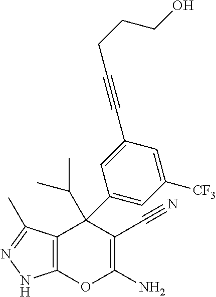 [SMILES: Cc1n[nH]c2c1C(c1cc(C#CCCCO)cc(C(F)(F)F)c1)(C(C)C)C(C#N)=C(N)O2] |
| 2 | 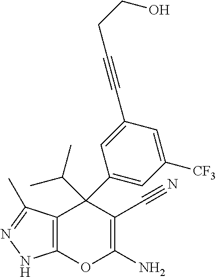 [SMILES: Cc1n[nH]c2c1C(c1cc(C#CCCO)cc(C(F)(F)F)c1)(C(C)C)C(C#N)=C(N)O2] |
| 3 | 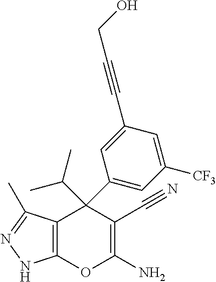 [SMILES: Cc1n[nH]c2c1C(c1cc(C#CCO)cc(C(F)(F)F)c1)(C(C)C)C(C#N)=C(N)O2] |
| 4 | 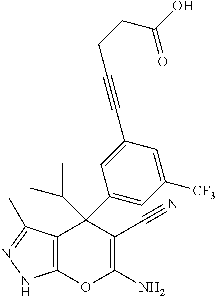 [SMILES: Cc1n[nH]c2c1C(c1cc(C#CCCC(=O)O)cc(C(F)(F)F)c1)(C(C)C)C(C#N)=C(N)O2] |
| 5 | 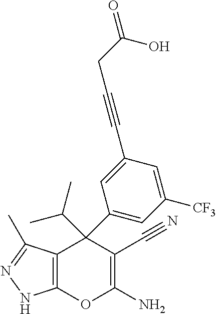 [SMILES: Cc1n[nH]c2c1C(c1cc(C#CCC(=O)O)cc(C(F)(F)F)c1)(C(C)C)C(C#N)=C(N)O2] |
| 6 | 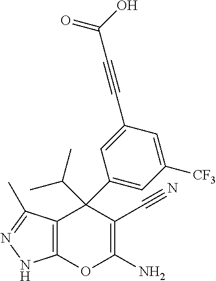 [SMILES: Cc1n[nH]c2c1C(c1cc(C#CC(=O)O)cc(C(F)(F)F)c1)(C(C)C)C(C#N)=C(N)O2] |
| 7 | 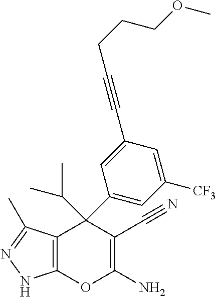 [SMILES: COCCCC#Cc1cc(C(F)(F)F)cc(C2(C(C)C)C(C#N)=C(N)Oc3[nH]nc(C)c32)c1] |
| 8 | 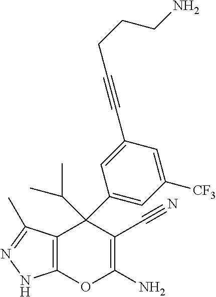 [SMILES: Cc1n[nH]c2c1C(c1cc(C#CCCCN)cc(C(F)(F)F)c1)(C(C)C)C(C#N)=C(N)O2] |
| 9 | 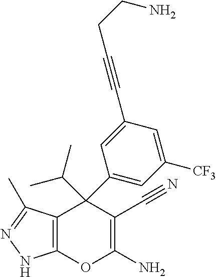 [SMILES: Cc1n[nH]c2c1C(c1cc(C#CCCN)cc(C(F)(F)F)c1)(C(C)C)C(C#N)=C(N)O2] |
| 10 | 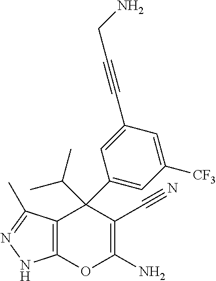 [SMILES: Cc1n[nH]c2c1C(c1cc(C#CCN)cc(C(F)(F)F)c1)(C(C)C)C(C#N)=C(N)O2] |
| 11 | 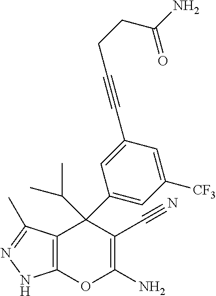 [SMILES: Cc1n[nH]c2c1C(c1cc(C#CCCC(N)=O)cc(C(F)(F)F)c1)(C(C)C)C(C#N)=C(N)O2] |
| 12 | 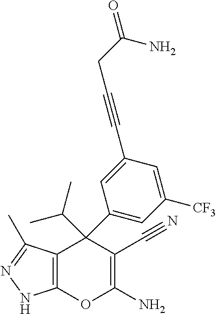 [SMILES: Cc1n[nH]c2c1C(c1cc(C#CCC(N)=O)cc(C(F)(F)F)c1)(C(C)C)C(C#N)=C(N)O2] |
| 13 | 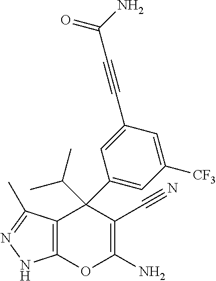 [SMILES: Cc1n[nH]c2c1C(c1cc(C#CC(N)=O)cc(C(F)(F)F)c1)(C(C)C)C(C#N)=C(N)O2] |
| 14 | 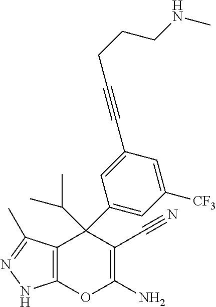 [SMILES: CNCCCC#Cc1cc(C(F)(F)F)cc(C2(C(C)C)C(C#N)=C(N)Oc3[nH]nc(C)c32)c1] |
| 15 | 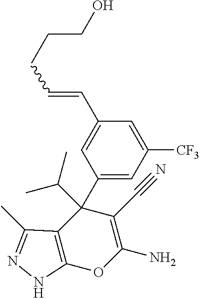 [SMILES: Cc1n[nH]c2c1C(c1cc(C=CCCCO)cc(C(F)(F)F)c1)(C(C)C)C(C#N)=C(N)O2] |
| 16 | 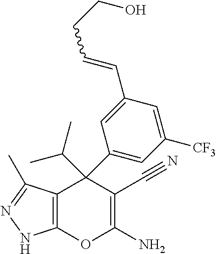 [SMILES: Cc1n[nH]c2c1C(c1cc(C=CCCO)cc(C(F)(F)F)c1)(C(C)C)C(C#N)=C(N)O2] |
| 17 | 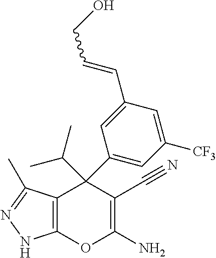 [SMILES: Cc1n[nH]c2c1C(c1cc(C=CCO)cc(C(F)(F)F)c1)(C(C)C)C(C#N)=C(N)O2] |
| 18 | 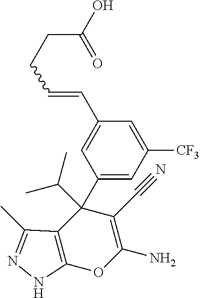 [SMILES: Cc1n[nH]c2c1C(c1cc(C=CCCC(=O)O)cc(C(F)(F)F)c1)(C(C)C)C(C#N)=C(N)O2] |
| 19 | 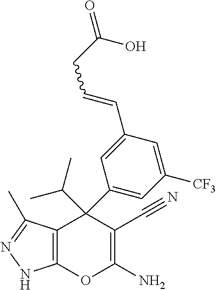 [SMILES: Cc1n[nH]c2c1C(c1cc(C=CCC(=O)O)cc(C(F)(F)F)c1)(C(C)C)C(C#N)=C(N)O2] |
| 20 | 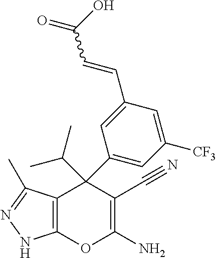 [SMILES: Cc1n[nH]c2c1C(c1cc(C=CC(=O)O)cc(C(F)(F)F)c1)(C(C)C)C(C#N)=C(N)O2] |
| 21 | 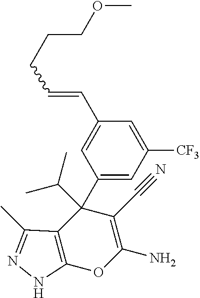 [SMILES: COCCCC=Cc1cc(C(F)(F)F)cc(C2(C(C)C)C(C#N)=C(N)Oc3[nH]nc(C)c32)c1] |
| 22 | 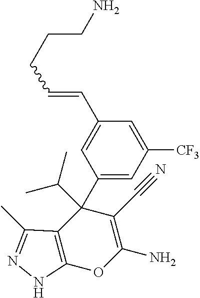 [SMILES: Cc1n[nH]c2c1C(c1cc(C=CCCCN)cc(C(F)(F)F)c1)(C(C)C)C(C#N)=C(N)O2] |
| 23 | 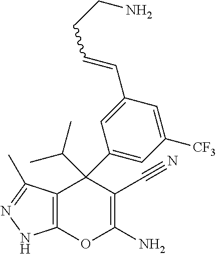 [SMILES: Cc1n[nH]c2c1C(c1cc(C=CCCN)cc(C(F)(F)F)c1)(C(C)C)C(C#N)=C(N)O2] |
| 24 | 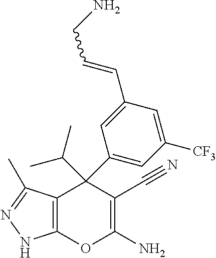 [SMILES: Cc1n[nH]c2c1C(c1cc(C=CCN)cc(C(F)(F)F)c1)(C(C)C)C(C#N)=C(N)O2] |
| 25 | 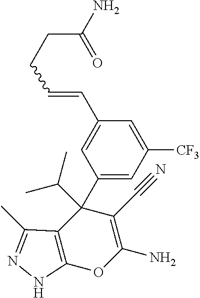 [SMILES: Cc1n[nH]c2c1C(c1cc(C=CCCC(N)=O)cc(C(F)(F)F)c1)(C(C)C)C(C#N)=C(N)O2] |
| 26 | 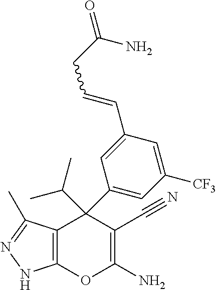 [SMILES: Cc1n[nH]c2c1C(c1cc(C=CCC(N)=O)cc(C(F)(F)F)c1)(C(C)C)C(C#N)=C(N)O2] |
| 27 | 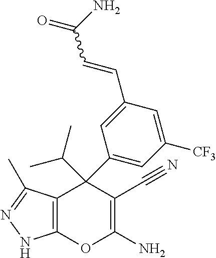 [SMILES: Cc1n[nH]c2c1C(c1cc(C=CC(N)=O)cc(C(F)(F)F)c1)(C(C)C)C(C#N)=C(N)O2] |
| 28 | 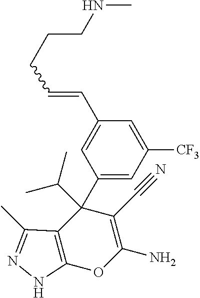 [SMILES: CNCCCC=Cc1cc(C(F)(F)F)cc(C2(C(C)C)C(C#N)=C(N)Oc3[nH]nc(C)c32)c1] |
| 29 | 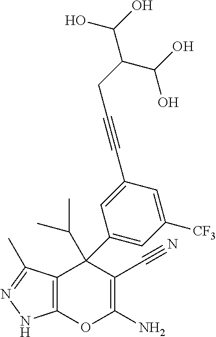 [SMILES: Cc1n[nH]c2c1C(c1cc(C#CCC(C(O)O)C(O)O)cc(C(F)(F)F)c1)(C(C)C)C(C#N)=C(N)O2] |
| 30 | 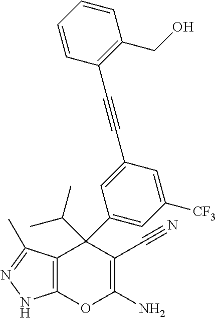 [SMILES: Cc1n[nH]c2c1C(c1cc(C#Cc3ccccc3CO)cc(C(F)(F)F)c1)(C(C)C)C(C#N)=C(N)O2] |
| 31 | 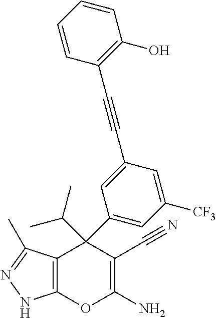 [SMILES: Cc1n[nH]c2c1C(c1cc(C#Cc3ccccc3O)cc(C(F)(F)F)c1)(C(C)C)C(C#N)=C(N)O2] |
| 32 | 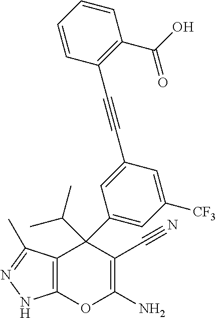 [SMILES: Cc1n[nH]c2c1C(c1cc(C#Cc3ccccc3C(=O)O)cc(C(F)(F)F)c1)(C(C)C)C(C#N)=C(N)O2] |
| 33 | 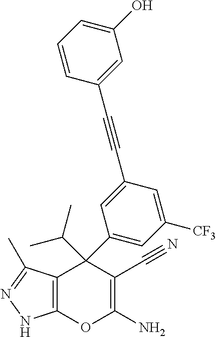 [SMILES: Cc1n[nH]c2c1C(c1cc(C#Cc3cccc(O)c3)cc(C(F)(F)F)c1)(C(C)C)C(C#N)=C(N)O2] |
| 34 | 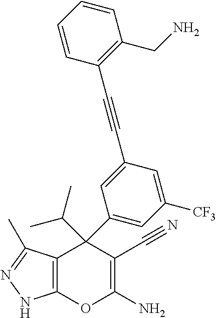 [SMILES: Cc1n[nH]c2c1C(c1cc(C#Cc3ccccc3CN)cc(C(F)(F)F)c1)(C(C)C)C(C#N)=C(N)O2] |
| 35 | 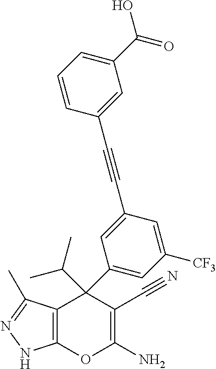 [SMILES: Cc1n[nH]c2c1C(c1cc(C#Cc3cccc(C(=O)O)c3)cc(C(F)(F)F)c1)(C(C)C)C(C#N)=C(N)O2] |
| 36 | 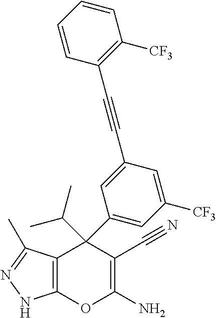 [SMILES: Cc1n[nH]c2c1C(c1cc(C#Cc3ccccc3C(F)(F)F)cc(C(F)(F)F)c1)(C(C)C)C(C#N)=C(N)O2] |
| 37 | 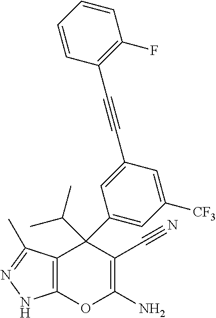 [SMILES: Cc1n[nH]c2c1C(c1cc(C#Cc3ccccc3F)cc(C(F)(F)F)c1)(C(C)C)C(C#N)=C(N)O2] |
| 38 | 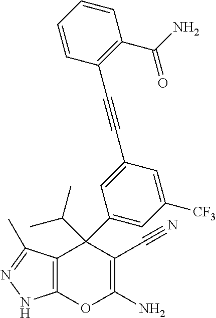 [SMILES: Cc1n[nH]c2c1C(c1cc(C#Cc3ccccc3C(N)=O)cc(C(F)(F)F)c1)(C(C)C)C(C#N)=C(N)O2] |
| 39 | 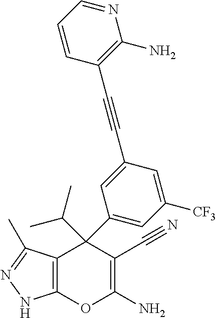 [SMILES: Cc1n[nH]c2c1C(c1cc(C#Cc3cccnc3N)cc(C(F)(F)F)c1)(C(C)C)C(C#N)=C(N)O2] |
| 40 | 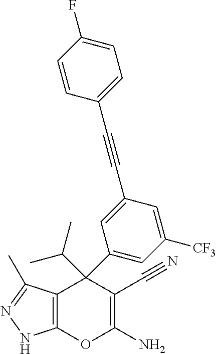 [SMILES: Cc1n[nH]c2c1C(c1cc(C#Cc3ccc(F)cc3)cc(C(F)(F)F)c1)(C(C)C)C(C#N)=C(N)O2] |
| 41 | 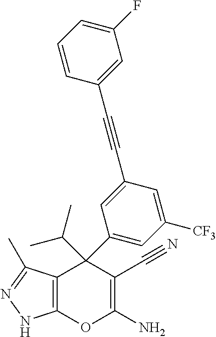 [SMILES: Cc1n[nH]c2c1C(c1cc(C#Cc3cccc(F)c3)cc(C(F)(F)F)c1)(C(C)C)C(C#N)=C(N)O2] |
| 42 | 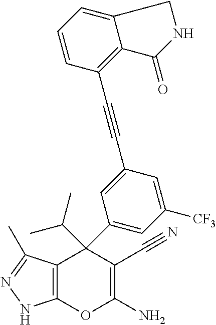 [SMILES: Cc1n[nH]c2c1C(c1cc(C#Cc3cccc4c3C(=O)NC4)cc(C(F)(F)F)c1)(C(C)C)C(C#N)=C(N)O2] |
| 43 | 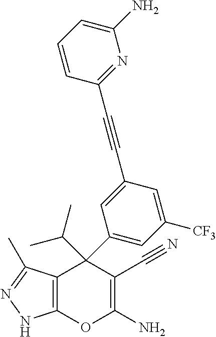 [SMILES: Cc1n[nH]c2c1C(c1cc(C#Cc3cccc(N)n3)cc(C(F)(F)F)c1)(C(C)C)C(C#N)=C(N)O2] |
| 44 | 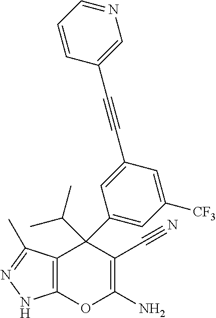 [SMILES: Cc1n[nH]c2c1C(c1cc(C#Cc3cccnc3)cc(C(F)(F)F)c1)(C(C)C)C(C#N)=C(N)O2] |
| 45 | 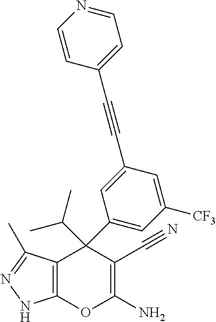 [SMILES: Cc1n[nH]c2c1C(c1cc(C#Cc3ccncc3)cc(C(F)(F)F)c1)(C(C)C)C(C#N)=C(N)O2] |
| 46 | 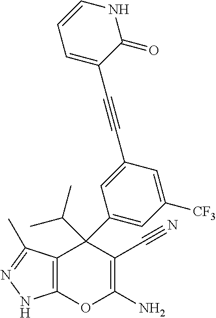 [SMILES: Cc1n[nH]c2c1C(c1cc(C#Cc3ccc[nH]c3=O)cc(C(F)(F)F)c1)(C(C)C)C(C#N)=C(N)O2] |
| 47 | 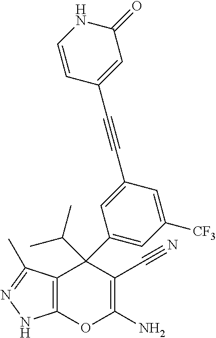 [SMILES: Cc1n[nH]c2c1C(c1cc(C#Cc3cc[nH]c(=O)c3)cc(C(F)(F)F)c1)(C(C)C)C(C#N)=C(N)O2] |
| 48 | 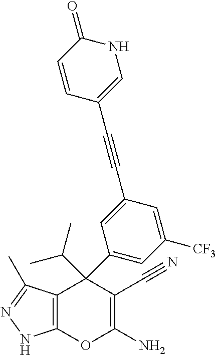 [SMILES: Cc1n[nH]c2c1C(c1cc(C#Cc3ccc(=O)[nH]c3)cc(C(F)(F)F)c1)(C(C)C)C(C#N)=C(N)O2] |
| 49 | 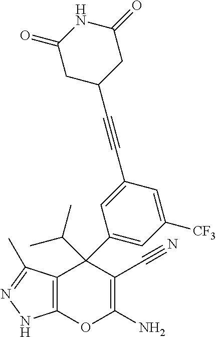 [SMILES: Cc1n[nH]c2c1C(c1cc(C#CC3CC(=O)NC(=O)C3)cc(C(F)(F)F)c1)(C(C)C)C(C#N)=C(N)O2] |
| 50 | 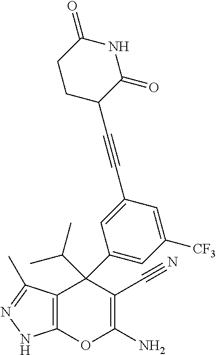 [SMILES: Cc1n[nH]c2c1C(c1cc(C#CC3CCC(=O)NC3=O)cc(C(F)(F)F)c1)(C(C)C)C(C#N)=C(N)O2] |
| 51 | 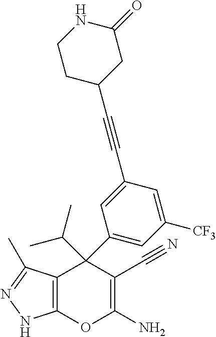 [SMILES: Cc1n[nH]c2c1C(c1cc(C#CC3CCNC(=O)C3)cc(C(F)(F)F)c1)(C(C)C)C(C#N)=C(N)O2] |
| 52 | 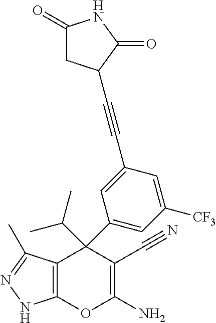 [SMILES: Cc1n[nH]c2c1C(c1cc(C#CC3CC(=O)NC3=O)cc(C(F)(F)F)c1)(C(C)C)C(C#N)=C(N)O2] |
| 53 | 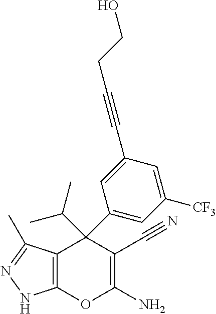 [SMILES: Cc1n[nH]c2c1C(c1cc(C#CCCO)cc(C(F)(F)F)c1)(C(C)C)C(C#N)=C(N)O2] |
| 54 | 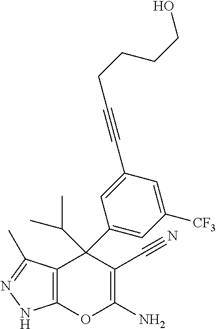 [SMILES: Cc1n[nH]c2c1C(c1cc(C#CCCCCO)cc(C(F)(F)F)c1)(C(C)C)C(C#N)=C(N)O2] |
| 55 | 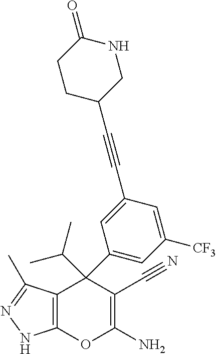 [SMILES: Cc1n[nH]c2c1C(c1cc(C#CC3CCC(=O)NC3)cc(C(F)(F)F)c1)(C(C)C)C(C#N)=C(N)O2] |
| 56 | 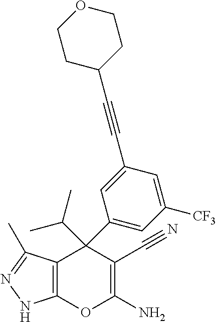 [SMILES: Cc1n[nH]c2c1C(c1cc(C#CC3CCOCC3)cc(C(F)(F)F)c1)(C(C)C)C(C#N)=C(N)O2] |
| 57 | 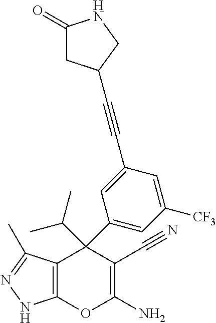 [SMILES: Cc1n[nH]c2c1C(c1cc(C#CC3CNC(=O)C3)cc(C(F)(F)F)c1)(C(C)C)C(C#N)=C(N)O2] |
| 58 | 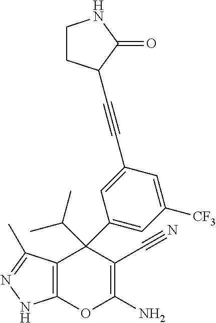 [SMILES: Cc1n[nH]c2c1C(c1cc(C#CC3CCNC3=O)cc(C(F)(F)F)c1)(C(C)C)C(C#N)=C(N)O2] |
| 59 | 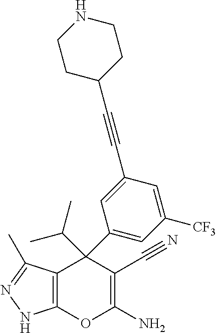 [SMILES: Cc1n[nH]c2c1C(c1cc(C#CC3CCNCC3)cc(C(F)(F)F)c1)(C(C)C)C(C#N)=C(N)O2] |
| 60 | 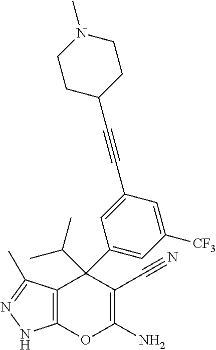 [SMILES: Cc1n[nH]c2c1C(c1cc(C#CC3CCN(C)CC3)cc(C(F)(F)F)c1)(C(C)C)C(C#N)=C(N)O2] |
| 61 | 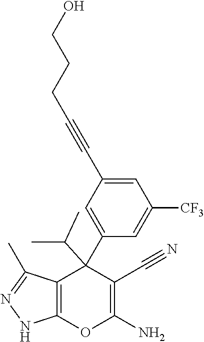 [SMILES: Cc1n[nH]c2c1C(c1cc(C#CCCCO)cc(C(F)(F)F)c1)(C(C)C)C(C#N)=C(N)O2] |
| 62 | 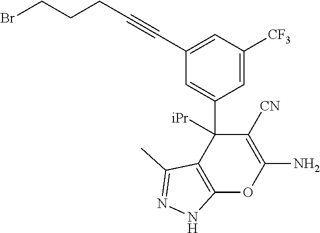 [SMILES: Cc1n[nH]c2c1C(c1cc(C#CCCCBr)cc(C(F)(F)F)c1)(C(C)C)C(C#N)=C(N)O2] |
| 63 | 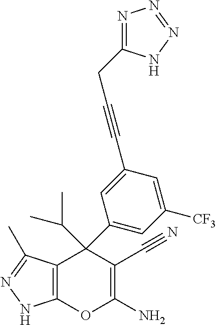 [SMILES: Cc1n[nH]c2c1C(c1cc(C#CCc3nnn[nH]3)cc(C(F)(F)F)c1)(C(C)C)C(C#N)=C(N)O2] |
| 64 | 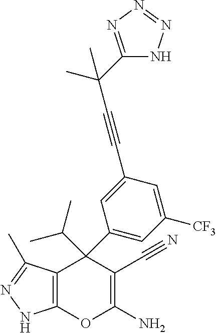 [SMILES: Cc1n[nH]c2c1C(c1cc(C#CC(C)(C)c3nnn[nH]3)cc(C(F)(F)F)c1)(C(C)C)C(C#N)=C(N)O2] |
| 65 | 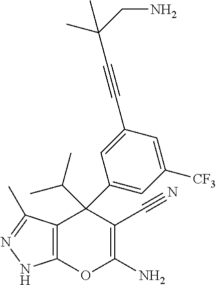 [SMILES: Cc1n[nH]c2c1C(c1cc(C#CC(C)(C)CN)cc(C(F)(F)F)c1)(C(C)C)C(C#N)=C(N)O2] |
| 66 | 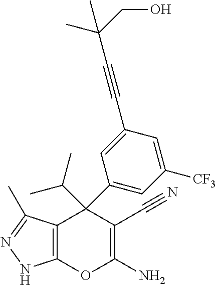 [SMILES: Cc1n[nH]c2c1C(c1cc(C#CC(C)(C)CO)cc(C(F)(F)F)c1)(C(C)C)C(C#N)=C(N)O2] |
| 67 | 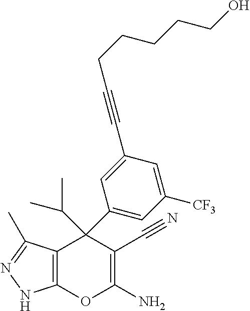 [SMILES: Cc1n[nH]c2c1C(c1cc(C#CCCCCCO)cc(C(F)(F)F)c1)(C(C)C)C(C#N)=C(N)O2] |
| 68 | 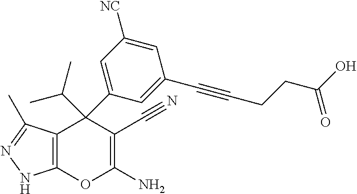 [SMILES: Cc1n[nH]c2c1C(c1cc(C#N)cc(C#CCCC(=O)O)c1)(C(C)C)C(C#N)=C(N)O2] |
| 69 | 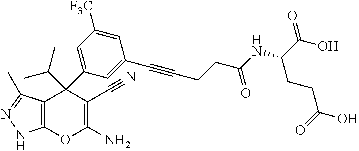 [SMILES: Cc1n[nH]c2c1C(c1cc(C#CCCC(=O)N[C@@H](CCC(=O)O)C(=O)O)cc(C(F)(F)F)c1)(C(C)C)C(C#N)=C(N)O2] |
| 70 | 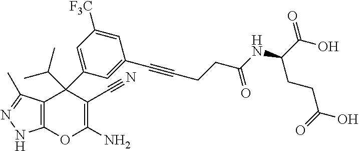 [SMILES: Cc1n[nH]c2c1C(c1cc(C#CCCC(=O)N[C@H](CCC(=O)O)C(=O)O)cc(C(F)(F)F)c1)(C(C)C)C(C#N)=C(N)O2] |
| 71 | 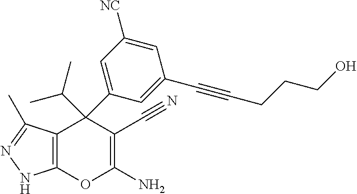 [SMILES: Cc1n[nH]c2c1C(c1cc(C#N)cc(C#CCCCO)c1)(C(C)C)C(C#N)=C(N)O2] |
| 72 | 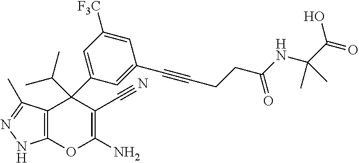 [SMILES: Cc1n[nH]c2c1C(c1cc(C#CCCC(=O)NC(C)(C)C(=O)O)cc(C(F)(F)F)c1)(C(C)C)C(C#N)=C(N)O2] |
| 73 | 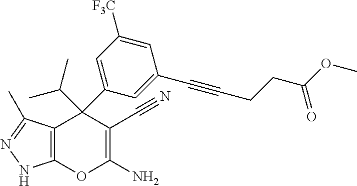 [SMILES: COC(=O)CCC#Cc1cc(C(F)(F)F)cc(C2(C(C)C)C(C#N)=C(N)Oc3[nH]nc(C)c32)c1] |
| 74 (SHIN2, KDG112) | 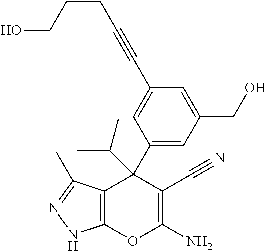 [SMILES: Cc1n[nH]c2c1C(c1cc(C#CCCCO)cc(CO)c1)(C(C)C)C(C#N)=C(N)O2] |

In one aspect, an SHMNT inhibitor is a compound of Formula IV:

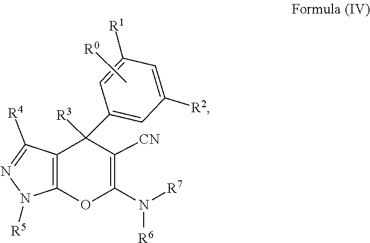

or a pharmaceutically acceptable salt thereof, wherein:

R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁴ is selected from H, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl; R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl, or R⁵ is selected from any of the foregoing and R⁶ and R⁷ taken together with the nitrogen atom to which they are attached form a substituted or unsubstituted 3-6 membered ring; with the proviso that the occurrences of R⁵, R⁶ and R⁷ are not all H simultaneously;

each occurrence of R⁵ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl, or a pharmaceutically acceptable salt thereof.

In certain embodiments, the compound of Formula (IV) is represented by Formula (IVa) (wherein the R groups are as described above for Formula (IV):

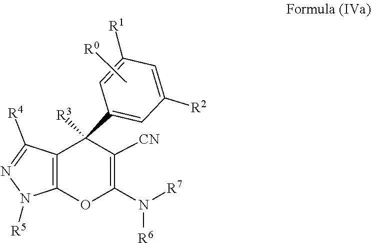

or a pharmaceutically acceptable salt thereof.

In certain embodiments, the compound of Formula (IV) is represented by Formula (IVb) (wherein the R groups are as described above for Formula (IV)):

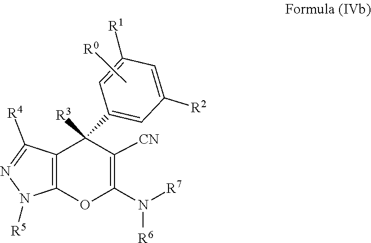

or a pharmaceutically acceptable salt thereof.

In certain embodiments of any of the foregoing or following, R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy.

In certain embodiments of any of the foregoing or following, R⁰ is selected from hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² (and, optionally R⁰) are each independently selected from —H, halogen, hydroxyl, nitro, nitrile, —OR¹¹, substituted or unsubstituted C₁-C₆ alkyl, substituted or unsubstituted C₁-C₆ haloalkyl, or substituted or unsubstituted C₁-C₆ haloalkoxy. In other embodiments, R¹ and R² (and, optionally R⁰) are each independently selected from —H, halogen, substituted or unsubstituted C₁-C₆ haloalkyl, substituted or unsubstituted C₁-C₆ haloalkoxy, or substituted or unsubstituted C₁-C₆ alkyl.

In certain embodiments of any of the foregoing or following, R¹ and R² (and, optionally R⁰) are each independently selected from —H, methoxy, fluoro, chloro, bromo, hydroxyl, nitro, nitrile, methyl, trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² (and, optionally R⁰) are each independently selected from —H, methoxy, chloro, nitro, nitrile, or trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² (and, optionally, R⁰) are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from substituted or unsubstituted C₁-C₆ alkyl, or substituted or unsubstituted cycloalkyl.

In certain embodiments of any of the foregoing or following, R³ is selected from methyl, ethyl, propyl, isopropyl, cyclopropyl, or cyclobutyl. In certain embodiments, any of the foregoing may be optionally substituted.

In certain embodiments of any of the foregoing or following, R³ is selected from isopropyl, cyclopropyl, or cyclobutyl. In certain embodiments, any of the foregoing may be optionally substituted.

In certain embodiments of any of the foregoing or following, R³ is cyclobutyl. In certain embodiments, acyclobutyl may be optionally substituted.

In certain embodiments of any of the foregoing or following, R⁴ is selected from —H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁴ is selected from —H, substituted or unsubstituted C₁-C₆ alkyl, substituted or unsubstituted cycloalkyl, substituted or unsubstituted aryl, or substituted or unsubstituted arylalkyl.

In certain embodiments of any of the foregoing or following, R⁴ is selected from methyl, ethyl, isopropyl, cyclopropyl, cyclobutyl, cyclopentyl, cyclohexyl, phenyl or benzyl. In certain embodiments, any of the foregoing may be optionally substituted.

In certain embodiments of any of the foregoing or following, R⁴ is methyl or isopropyl. In certain embodiments, any of the foregoing may be optionally substituted.

In certain embodiments of any of the foregoing or following, R⁴ is methyl. In certain embodiments, methyl may be optionally substituted.

In certain embodiments of any of the foregoing or following, R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, substituted or unsubstituted arylalkyl, or R⁵ is selected from any of the foregoing and R⁶ and R⁷ taken together with the nitrogen atom to which they are attached form a substituted or unsubstituted 3-6 membered ring.

In certain embodiments of any of the foregoing or following, R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, or substituted or unsubstituted arylalkyl.

In certain embodiments of any of the foregoing or following, R⁵, R⁶ and R⁷ are each independently selected from —H, methyl, ethyl, isopropyl, cyclopropyl, cyclobutyl, cyclopentyl, cyclohexyl, phenyl, benzyl, or —COCH₃. In certain embodiments, any of the foregoing, except —H, may be optionally substituted.

In certain embodiments of any of the foregoing or following, R⁵, R⁶ and R⁷ are each independently selected from —H, methyl, phenyl, or —COCH₃. In certain embodiments, any of the foregoing, except —H, may be optionally substituted.

In certain embodiments of any of the foregoing or following, R⁵ and R⁶ are each independently selected from —H, methyl or phenyl. In certain embodiments, any of the foregoing, except —H, may be optionally substituted.

In certain embodiments of any of the foregoing or following, R⁷ is —H.

In certain embodiments of any of the foregoing or following, R⁶ and R⁷ taken together with the nitrogen atom to which they are attached form a substituted or unsubstituted 3-6 membered ring. In certain embodiments the 3-6 membered ring is a monocylic ring. In certain embodiments, the 3-6 membered ring may be saturated or unsaturated (e.g., contain at least one double bond). In certain embodiments, the 3-6 membered ring may contain one or two additional heteroatoms, other than the nitrogen atom to which R⁶ and R⁷ are attached.

In certain embodiments of any of the foregoing or following, R⁵ is selected from —H, methyl, ethyl, isopropyl, cyclopropyl, cyclobutyl, cyclopentyl, cyclohexyl, phenyl, benzyl, or —COCH₃, and R⁶ and R⁷ taken together with the nitrogen atom to which they are attached form a substituted or unsubstituted ring selected from:

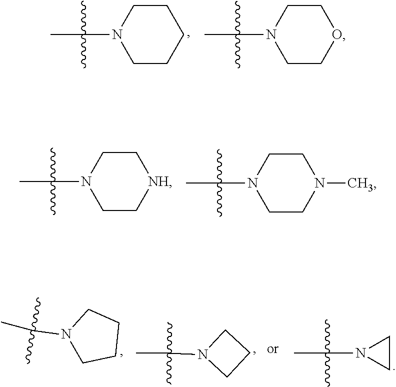

In certain embodiments of any of the foregoing or following, R⁵ is selected from —H, methyl, phenyl, or —COCH₃, and R⁶ and R⁷ taken together with the nitrogen atom to which they are attached form a substituted or unsubstituted ring selected from:

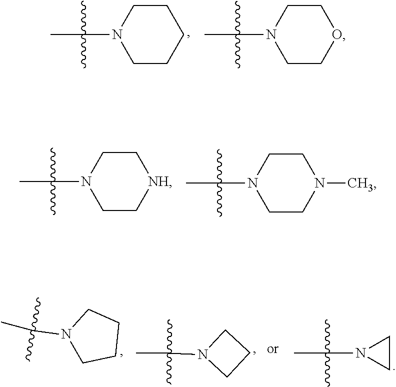

In certain embodiments of any of the foregoing or following, each occurrence of R¹¹ is independently selected from substituted or unsubstituted C₁-C₆ alkyl or substituted or unsubstituted cycloalkyl. In certain embodiments, there is no occurrence of R¹¹.

In certain embodiments of any of the foregoing or following, each occurrence of R¹⁰ and R¹² is each independently selected from —H, substituted or unsubstituted C₁-C₆ alkyl, or substituted or unsubstituted cycloalkyl, such as from —H, or substituted or unsubstituted C₁-C₆ alkyl. In certain embodiments, each occurrence of R¹⁰ and R¹² is —H. In certain embodiments, there is no occurrence of R¹⁰ and/or R¹².

In certain embodiments of any of the foregoing or following, R⁰ is selected from —H, halogen, hydroxyl, nitro, nitrile, —S(O)₂R¹¹, —S(O)₂NR¹⁰R¹², —OR¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R¹ and R² are each independently selected from —H, methoxy, fluoro, chloro, bromo, hydroxyl, nitro, nitrile, methyl, or trifluoromethyl;

R³ is selected from methyl, ethyl, propyl, isopropyl, cyclopropyl, or cyclobutyl;

R⁴ is selected from methyl, ethyl, isopropyl, cyclopropyl, cyclobutyl, cyclopentyl, cyclohexyl, phenyl or benzyl; and

R⁵, R⁶ and R⁷ are each independently selected from —H, methyl, ethyl, isopropyl, cyclopropyl, cyclobutyl, cyclopentyl, cyclohexyl, phenyl, benzyl, or —COCH₃.

In certain embodiments of any of the foregoing or following, R⁰ is selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹²;

R¹ and R² are each independently selected from —H, methoxy, chloro, nitro, nitrile, or trifluoromethyl;

R³ is selected from isopropyl, cyclopropyl, or cyclobutyl;

R⁴ is methyl or isopropyl; and

R⁵, R⁶ and R⁷ are each independently selected from —H, methyl, phenyl, or —COCH₃.

In certain embodiments of any of the foregoing or following, R⁰ is selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹²;

R¹ and R² are each —CF₃;

R³ is cyclobutyl;

R⁴ is methyl;

R⁵ and R⁶ are each independently selected from H, methyl or phenyl; and

R⁷ is H.

In certain embodiments, the compound is selected from the group consisting of:

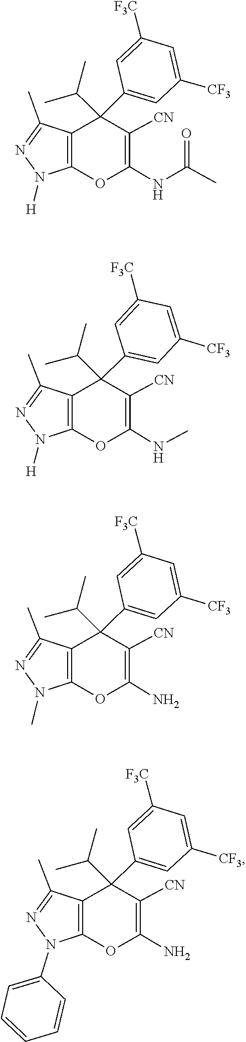 [SMILES: CC(=O)NC1=C(C#N)C(c2cc(C(F)(F)F)cc(C(F)(F)F)c2)(C(C)C)c2c(C)n[nH]c2O1 ; CNC1=C(C#N)C(c2cc(C(F)(F)F)cc(C(F)(F)F)c2)(C(C)C)c2c(C)n[nH]c2O1 ; Cc1nn(C)c2c1C(c1cc(C(F)(F)F)cc(C(F)(F)F)c1)(C(C)C)C(C#N)=C(N)O2 ; Cc1nn(-c2ccccc2)c2c1C(c1cc(C(F)(F)F)cc(C(F)(F)F)c1)(C(C)C)C(C#N)=C(N)O2]

or a pharmaceutically acceptable salt of any of the foregoing.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and R² are not —H.

In one aspect, an SHMT inhibitor is a compound of Formula (V):

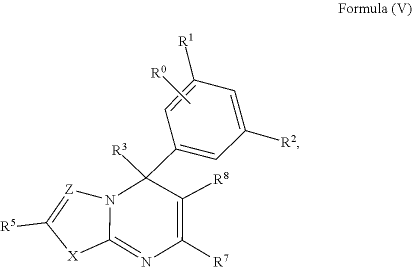

or a pharmaceutically acceptable salt thereof, wherein:

Z is N or CR⁴;

X is O, S, CH₂, or NR⁶;

R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁴ is selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

R⁵ is selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl, or R⁵ and R⁴ taken together with the respective carbon atom to which they are attached form a substituted or unsubstituted 4-12 membered ring;

R⁶ is selected from H, substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, or substituted or unsubstituted heteroarylalkyl;

R⁷ and R⁸ are each independently selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

each occurrence of R¹¹ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and

each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments, the compounds of Formula (V) are represented by Formula (Va) (wherein the R groups are as described above for Formula (V)):

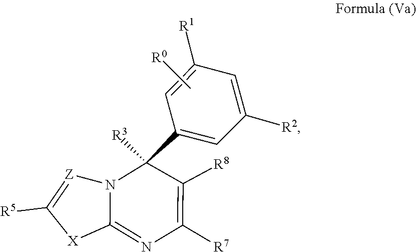

or a pharmaceutically acceptable salt thereof.

In certain embodiments, the compounds of Formula (V) are represented Formula (Vb) (wherein the R groups are as described above for Formula (V)):

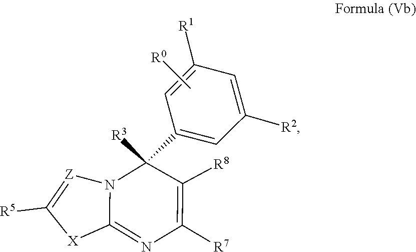

or a pharmaceutically acceptable salt thereof.

In certain embodiments of any of the foregoing or following, R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy.

In certain embodiments of any of the foregoing or following, R⁰ is selected from hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, halogen, hydroxyl, nitro, nitrile, —OR¹¹, substituted or unsubstituted C₁-C₆ alkyl, substituted or unsubstituted C₁-C₆ haloalkyl, substituted or unsubstituted C₁-C₆ haloalkoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, methoxy, fluoro, chloro, bromo, hydroxyl, nitro, nitrile, methyl, trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from methyl, ethyl, propyl, isopropyl, cyclopropyl, or cyclobutyl. In certain embodiments, any of the foregoing may be optionally substituted.

In certain embodiments of any of the foregoing or following, R³ is isopropyl or cyclopropyl. In certain embodiments, any of the foregoing may be optionally substituted.

In certain embodiments of any of the foregoing or following, R⁴ and R⁵ are each independently selected from —H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁴ and R⁵ are each independently selected from —H, substituted or unsubstituted C₁-C₆ alkyl, substituted or unsubstituted cycloalkyl, substituted or unsubstituted aryl, or substituted or unsubstituted arylalkyl.

In certain embodiments of any of the foregoing or following, R⁴ and R⁵ are each independently selected from —H, methyl, ethyl, propyl, isopropyl, cyclopropyl, cyclobutyl, cyclopentyl, cyclohexyl, phenyl or benzyl. In certain embodiments, any of the foregoing, except —H, may be optionally substituted.

In certain embodiments of any of the foregoing or following, R⁴ and R⁵ are independently —H or methyl. In certain embodiments, methyl may be optionally substituted.

In certain embodiments of any of the foregoing or following, R⁵ and R⁴ taken together with the respective carbon atom to which they are attached form a 4-12 membered ring selected from substituted or unsubstituted aryl, or substituted or unsubstituted heteroaryl.

In certain embodiments of any of the foregoing or following, R⁵ and R⁴ taken together with the respective carbon atom to which they are attached form a substituted or unsubstituted 4-12 membered ring containing 0-4 heteroatoms (0, 1, 2, 3 or 4) independently selected from N, O, or S.

In certain embodiments of any of the foregoing or following, the 4-12 membered ring is a monocyclic ring. In certain embodiments of any of the foregoing or following, the 4-12 membered ring is a polycyclic ring. In certain embodiments of any of the foregoing or following, the 4-12 membered ring is a bicyclic ring. In certain embodiments of any of the foregoing or following, when the 4-12 membered ring is a polycyclic ring, each ring is independently selected from saturated or unsaturated, and each ring may independently contain one or more heteroatoms (e.g., for a total of 1, 2, 3, 4 or 4 heteroatoms).

In certain embodiments of any of the foregoing or following, R⁵ and R⁴ taken together with the respective carbon atom to which they are attached form a phenyl ring. In certain embodiments, the phenyl ring may be optionally substituted.

In certain embodiments of any of the foregoing or following, R⁶ is selected from H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁶ is selected from —H, methyl, ethyl, isopropyl, cyclopropyl, cyclobutyl, cyclopentyl, cyclohexyl, phenyl, pyridyl, or benzyl. In certain embodiments, any of the foregoing may be optionally substituted.

In certain embodiments of any of the foregoing or following, R⁶ is —H.

In certain embodiments of any of the foregoing or following, R⁷ and R⁸ are each independently selected from —H, —NR¹⁰R¹², —C(O)NR¹⁰R¹², —N(R¹²)C(O)R¹¹, nitrile, methyl, ethyl, isopropyl, cyclopropryl, cyclobutyl, cyclopentyl, cyclohexyl, phenyl, or benzyl.

In certain embodiments of any of the foregoing or following, R⁷ is selected from —H, —NH₂, methyl, or phenyl. In certain embodiments, any of the foregoing except —H, may be optionally substituted.

In certain embodiments of any of the foregoing or following, R⁸ is selected from —H, nitrile, or —C(O)NH₂. In certain embodiments, —C(O)NH₂ may be optionally substituted.

In certain embodiments of any of the foregoing or following, each occurrence of R¹¹ is independently selected from substituted or unsubstituted C₁-C₆ alkyl or substituted or unsubstituted cycloalkyl. In certain embodiments, there is no occurrence of R¹¹.

In certain embodiments of any of the foregoing or following, each occurrence of R¹⁰ and R¹² is each independently selected from —H, substituted or unsubstituted C₁-C₆ alkyl, or substituted or unsubstituted cycloalkyl, such as from —H, or substituted or unsubstituted C₁-C₆ alkyl. In certain embodiments, each occurrence of R¹⁰ and R¹² is —H. In certain embodiments, there is no occurrence of R¹⁰ and/or R¹².

In certain embodiments of any of the foregoing or following, the disclosure provides a pharmaceutically acceptable salt thereof or an enantiomer thereof.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and R² are not —H.

In certain embodiments, when Z is N, and R⁸ is H, R⁷ cannot be substituted or unsubstituted aryl.

In certain embodiments, when R⁴ and R⁵ taken together with the respective carbon atoms to which they are attached for a substituted aryl ring, R⁸ is not —H, —OH, or —CN.

In certain embodiments, the compound of Formulae (V), (Va), or (Vb) is:

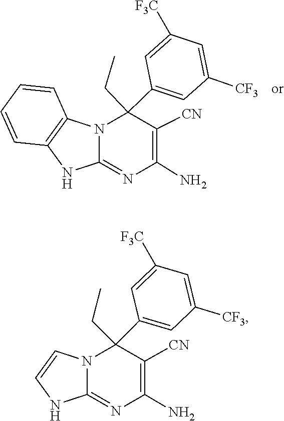 [SMILES: CCC1(c2cc(C(F)(F)F)cc(C(F)(F)F)c2)C(C#N)=C(N)N=C2Nc3ccccc3N21 ; CCC1(c2cc(C(F)(F)F)cc(C(F)(F)F)c2)C(C#N)=C(N)N=C2NC=CN21]

or a pharmaceutically acceptable salt thereof.

In one aspect, an SHMT inhibitor is a compound represented by Formula (VI):

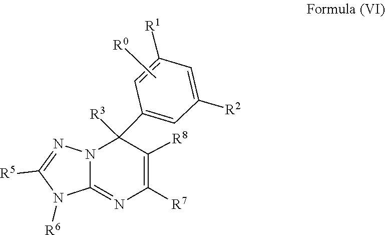

or a pharmaceutically acceptable salt thereof, wherein:

R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁵ is selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

R⁶ is selected from —H, substituted or unsubstituted C₁-C₆ alkyl, substituted or unsubstituted cycloalkyl, substituted or unsubstituted heterocyclyl, substituted or unsubstituted aryl, substituted or unsubstituted heteroaryl substituted or unsubstituted arylalkyl, or substituted or unsubstituted heteroarylalkyl;

R⁷ and R⁸ are each independently selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

each occurrence of R¹¹ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and

each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments, the compounds of Formula (VI) are represented by Formula (VIa) (wherein the R groups are as described above for Formula (VI)):

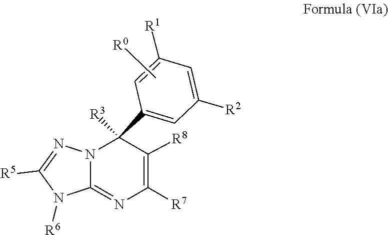

or a pharmaceutically acceptable salt thereof.

In certain embodiments, the compounds of Formula (VI) are represented by Formula (VIb) (wherein the R groups are as described above for Formula (VI)):

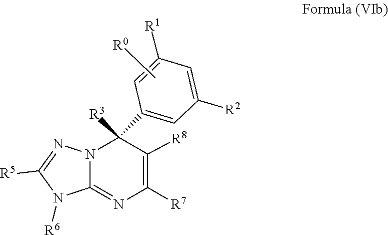

or a pharmaceutically acceptable salt thereof.

In certain embodiments of any of the foregoing or following, R⁰, R¹ and R² are each independently selected from —H, halogen, hydroxyl, nitro, nitrile, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹², substituted or unsubstituted C₁-C₆ alkyl, substituted or unsubstituted cycloalkyl, substituted or unsubstituted C₁-C₆ haloalkyl, or substituted or unsubstituted C₁-C₆ haloalkoxy.

In certain embodiments of any of the foregoing or following, R⁰ is selected from hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, halogen, hydroxyl, nitro, nitrile, —OR¹¹, substituted or unsubstituted C₁-C₆ alkyl, substituted or unsubstituted C₁-C₆ haloalkyl, or substituted or unsubstituted C₁-C₆ haloalkoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, methoxy, fluoro, chloro, bromo, hydroxyl, nitro, nitrile, methyl, trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from methyl, ethyl, propyl, isopropyl, cyclopropyl, or cyclobutyl. In certain embodiments, any of the foregoing may be optionally substituted.

In certain embodiments of any of the foregoing or following, R³ is isopropyl or cyclopropyl. In certain embodiments, any of the foregoing may be optionally substituted.

In certain embodiments of any of the foregoing or following, R⁵ is selected from —H, methyl, ethyl, propyl, isopropyl, cyclopropyl, cyclobutyl, cyclopentyl, cyclohexyl, phenyl or benzyl. In certain embodiments, any of the foregoing except —H, may be optionally substituted.

In certain embodiments of any of the foregoing or following, R⁵ is —H.

In certain embodiments of any of the foregoing or following, R⁶ is selected from H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁶ is selected from —H, methyl, ethyl, isopropyl, cyclopropyl, cyclobutyl, cyclopentyl, cyclohexyl, phenyl, pyridyl or benzyl. In certain embodiments, any of the foregoing except —H, may be optionally substituted.

In certain embodiments of any of the foregoing or following, R⁶ is —H.

In certain embodiments of any of the foregoing or following, R⁷ and R⁸ are each independently selected from —H, —NR¹⁰R¹², —C(O)NR¹⁰R¹², —N(R¹²)C(O)R¹¹, nitrile, methyl, ethyl, isopropyl, cyclopropryl, cyclobutyl, cyclopentyl, cyclohexyl, phenyl, or benzyl.

In certain embodiments of any of the foregoing or following, R⁷ is selected from —NH₂, methyl, or phenyl. In certain embodiments, any of the foregoing, may be optionally substituted.

In certain embodiments of any of the foregoing or following, R⁸ is selected from —H, nitrile, or —C(O)NH₂. In certain embodiments, —C(O)NH₂ may be optionally substituted.

In certain embodiments of any of the foregoing or following, each occurrence of R¹¹ is independently selected from substituted or unsubstituted C₁-C₆ alkyl or substituted or unsubstituted cycloalkyl. In certain embodiments, there is no occurrence of R¹¹.

In certain embodiments of any of the foregoing or following, each occurrence of R¹⁰ and R¹² is each independently selected from —H, substituted or unsubstituted C₁-C₆ alkyl, or substituted or unsubstituted cycloalkyl, such as from —H, or substituted or unsubstituted C₁-C₆ alkyl. In certain embodiments, each occurrence of R¹⁰ and R¹² is —H. In certain embodiments, there is no occurrence of R¹⁰ and/or R¹².

In certain embodiments of any of the foregoing or following, R⁰ is selected from —H, halogen, hydroxyl, nitro, nitrile, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹², substituted or unsubstituted C₁-C₆ alkyl, substituted or unsubstituted C₁-C₆ haloalkyl, or substituted or unsubstituted C₁-C₆ haloalkoxy;

R¹ and R² are each independently selected from —H, methoxy, fluoro, chloro, bromo, hydroxyl, nitro, nitrile, methyl, or trifluoromethyl;

R³ is selected from methyl, ethyl, propyl, isopropyl, cyclopropyl, or cyclobutyl;

R⁵ is selected from —H, methyl, ethyl, propyl or isopropyl;

R⁶ is selected from —H, methyl, ethyl, isopropyl, cyclopropyl, cyclobutyl, cyclopentyl, cyclohexyl, phenyl or benzyl; and

R⁷ and R⁸ are each independently selected from —H, —NH₂, —C(O)NH₂, nitrile, methyl, ethyl, isopropyl, cyclopropryl, cyclobutyl, cyclopentyl, cyclohexyl, phenyl, or benzyl.

In certain embodiments of any of the foregoing or following, R⁰ is selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹²;

R¹ and R² are each trifluoromethyl;

R³ is isopropyl or cyclopropyl;

R⁵ is —H;

R⁶ is —H;

R⁷ is selected from —NH₂, methyl, or phenyl; and

R⁸ is selected from H, nitrile, or —C(O)NH₂.

In certain embodiments, the compound is selected from:

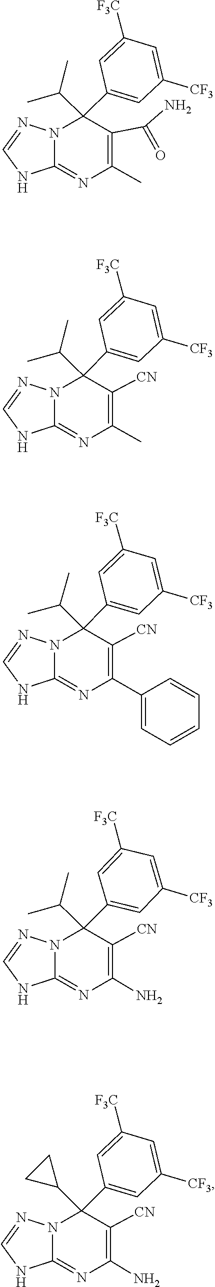 [SMILES: CC1=C(C(N)=O)C(c2cc(C(F)(F)F)cc(C(F)(F)F)c2)(C(C)C)N2N=CNC2=N1 ; CC1=C(C#N)C(c2cc(C(F)(F)F)cc(C(F)(F)F)c2)(C(C)C)N2N=CNC2=N1 ; CC(C)C1(c2cc(C(F)(F)F)cc(C(F)(F)F)c2)C(C#N)=C(c2ccccc2)N=C2NC=NN21 ; CC(C)C1(c2cc(C(F)(F)F)cc(C(F)(F)F)c2)C(C#N)=C(N)N=C2NC=NN21 ; N#CC1=C(N)N=C2NC=NN2C1(c1cc(C(F)(F)F)cc(C(F)(F)F)c1)C1CC1]

or a pharmaceutically acceptable salt of any of the foregoing.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and R² are not —H.

In certain embodiments, when R¹ is H, R⁷ is not a substituted or unsubstituted aryl.

In one aspect, an SHMT inhibitor is a compound of Formula (VII):

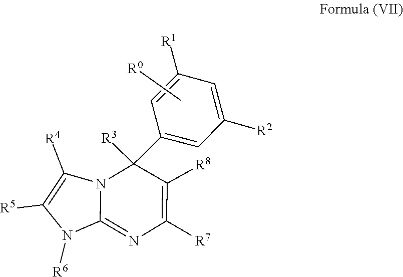

or a pharmaceutically acceptable salt thereof, wherein:

R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁴ and R⁵ are independently selected from H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl, or R⁵ and R⁴ taken together with the respective carbon atom to which they are attached form a substituted or unsubstituted 4-12 membered ring;

R⁶ is selected from —H, substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, or substituted or unsubstituted heteroarylalkyl;

R⁷ and R⁸ are each independently selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

each occurrence of R¹¹ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and

each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl, or a pharmaceutically acceptable salt thereof.

In certain embodiments of any of the foregoing or following, the compounds of Formula (VII) are represented by Formula (VIIa) (wherein the R groups are as described above for Formula (VII)):

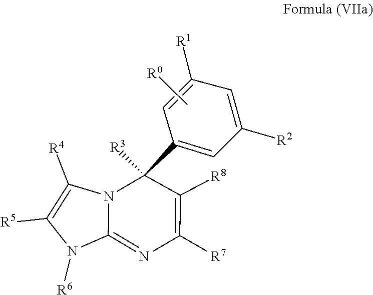

or a pharmaceutically acceptable salt thereof.

In certain embodiments of any of the foregoing or following, the compounds of Formula (VII) are represented by Formula (VIIb) (wherein the R groups are as described above for Formula (VII)):

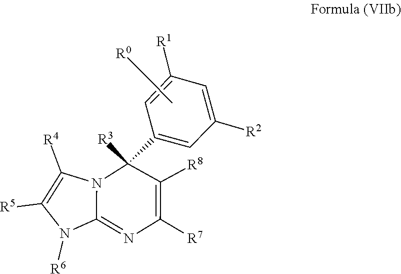

or a pharmaceutically acceptable salt thereof.

In certain embodiments of any of the foregoing or following, R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy.

In certain embodiments of any of the foregoing or following, R⁰ is selected from hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹².

In certain embodiments of any of the foregoing or following, R¹ and R² are each independently selected from —H, methoxy, fluoro, chloro, bromo, hydroxyl, nitro, nitrile, methyl, trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from methyl, ethyl, propyl, isopropyl, cyclopropyl, or cyclobutyl. In certain embodiments, any of the foregoing, may be optionally substituted.

In certain embodiments of any of the foregoing or following, R³ is isopropyl. In certain embodiments, isopropyl may be optionally substituted.

In certain embodiments of any of the foregoing or following, R⁴ and R⁵ are independently selected from H, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl.

In certain embodiments of any of the foregoing or following, R⁴ and R⁵ are each independently selected from —H, methyl, ethyl, propyl, isopropyl, cyclopropyl, cyclobutyl, cyclopentyl, cyclohexyl, phenyl or benzyl. In certain embodiments, any of the foregoing except —H, may be optionally substituted.

In certain embodiments of any of the foregoing or following, R⁴ and R⁵ are each independently —H or methyl. In certain embodiments, methyl may be optionally substituted.

In certain embodiments of any of the foregoing or following, R⁵ and R⁴ taken together with the respective carbon atoms to which they are attached form a substituted or unsubstituted 4-12 membered ring.

In certain embodiments of any of the foregoing or following, R⁵ and R⁴ taken together with the respective carbon atom to which they are attached form a 4-12 membered ring selected from substituted or unsubstituted aryl or substituted or unsubstituted heteroaryl.

In certain embodiments of any of the foregoing or following, R⁵ and R⁴ taken together with the respective carbon atom to which they are attached form a substituted or unsubstituted 4-12 membered ring containing 0-4 heteroatoms (0, 1, 2, 3 or 4) independently selected from N, O, or S.

In certain embodiments of any of the foregoing or following, the 4-12 membered ring is a monocyclic ring. In certain embodiments of any of the foregoing or following, the 4-12 membered ring is a polycyclic ring. In certain embodiments of any of the foregoing or following, the 4-12 membered ring is a bicyclic ring. In certain embodiments of any of the foregoing or following, when the 4-12 membered ring is a polycyclic ring, each ring is independently selected from saturated or unsaturated, and each ring may independently contain one or more heteroatoms.

In certain embodiments of any of the foregoing or following, R⁵ and R⁴ taken together with the respective carbon atom to which they are attached form a phenyl ring. In certain embodiments, the phenyl ring may be optionally substituted.

In certain embodiments of any of the foregoing or following, R⁶ is selected from H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁶ is selected from —H, methyl, ethyl, isopropyl, cyclopropyl, cyclobutyl, cyclopentyl, cyclohexyl, phenyl, pyridyl, or benzyl. In certain embodiments, any of the foregoing except —H, may be optionally substituted.

In certain embodiments of any of the foregoing or following, R⁶ is —H.

In certain embodiments of any of the foregoing or following, R⁷ and R⁸ are each independently selected from —H, —NR¹⁰R¹², —C(O)NR¹⁰R¹², —N(R¹²)C(O)R¹¹, nitrile, methyl, ethyl, isopropyl, cyclopropryl, cyclobutyl, cyclopentyl, cyclohexyl, phenyl, or benzyl.

In certain embodiments of any of the foregoing or following, R⁷ is selected from —H, —NH₂, methyl, or phenyl. In certain embodiments, any of the foregoing except —H, may be optionally substituted.

In certain embodiments of any of the foregoing or following, R⁸ is selected from —H, nitrile, or —C(O)NH₂. In certain embodiments, —C(O)NH₂ may be optionally substituted.

In certain embodiments of any of the foregoing or following, each occurrence of R¹¹ is independently selected from substituted or unsubstituted C₁-C₆ alkyl or substituted or unsubstituted cycloalkyl. In certain embodiments, there is no occurrence of R¹¹.

In certain embodiments of any of the foregoing or following, each occurrence of R¹⁰ and R¹² is each independently selected from —H, substituted or unsubstituted C₁-C₆ alkyl, or substituted or unsubstituted cycloalkyl, such as from —H, or substituted or unsubstituted C₁-C₆ alkyl. In certain embodiments, each occurrence of R¹⁰ and R¹² is —H. In certain embodiments, there is no occurrence of R¹⁰ and/or R¹².

In certain embodiments, the compound is selected from:

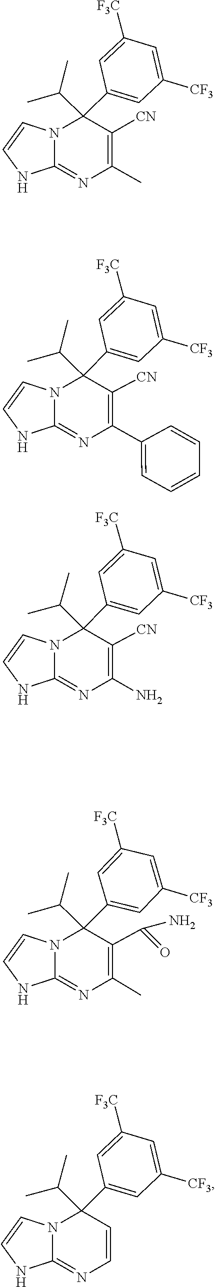 [SMILES: CC1=C(C#N)C(c2cc(C(F)(F)F)cc(C(F)(F)F)c2)(C(C)C)N2C=CNC2=N1 ; CC(C)C1(c2cc(C(F)(F)F)cc(C(F)(F)F)c2)C(C#N)=C(c2ccccc2)N=C2NC=CN21 ; CC(C)C1(c2cc(C(F)(F)F)cc(C(F)(F)F)c2)C(C#N)=C(N)N=C2NC=CN21 ; CC1=C(C(N)=O)C(c2cc(C(F)(F)F)cc(C(F)(F)F)c2)(C(C)C)N2C=CNC2=N1 ; CC(C)C1(c2cc(C(F)(F)F)cc(C(F)(F)F)c2)C=CN=C2NC=CN21]

or a pharmaceutically acceptable salt of any of the foregoing.

In certain embodiments, the compound of Formula (VII), (VIIa), or (VIIb) is:

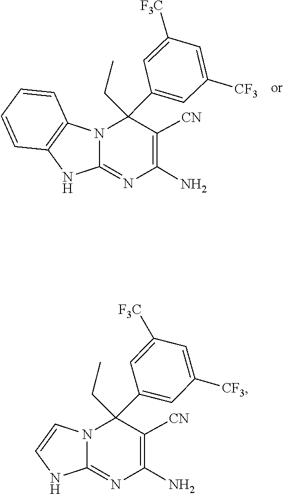 [SMILES: CCC1(c2cc(C(F)(F)F)cc(C(F)(F)F)c2)C(C#N)=C(N)N=C2Nc3ccccc3N21 ; CCC1(c2cc(C(F)(F)F)cc(C(F)(F)F)c2)C(C#N)=C(N)N=C2NC=CN21]

or a pharmaceutically acceptable salt of either of the foregoing.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and R² are not —H.

In one aspect, the disclosure provides compounds represented by general Formula (VIII):

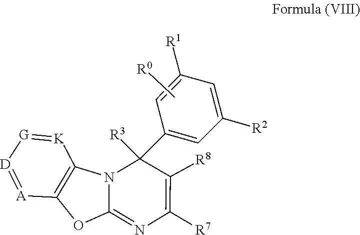

or a pharmaceutically acceptable salt thereof, wherein:

A, D, G and K are each independently N or CR¹⁵, provided that no more than two of A, D, G, and K are N simultaneously;

R⁰, R¹ and R² are each independently selected from —H, halogen, hydroxyl, nitro, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹² substituted or unsubstituted C₁-C₆ alkyl, substituted or unsubstituted cycloalkyl, substituted or unsubstituted heterocyclyl, substituted or unsubstituted aryl, substituted or unsubstituted heteroaryl substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁷ and R⁸ are each independently selected from —H, —NR¹⁰R¹², —C(O)NR¹⁰R¹², —N(R¹²)C(O)R¹¹, nitrile, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

each occurrence of R¹¹ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl;

each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and each occurrence of R¹⁵ is independently selected from —H, halogen, hydroxyl, nitro, nitrile, substituted or unsubstituted C₁-C₆ alkoxy, substituted or unsubstituted C₁-C₆ alkyl, or substituted or unsubstituted C₁-C₆ haloalkyl, substituted or unsubstituted C₁-C₆ haloalkoxy, or a pharmaceutically acceptable salt thereof.

In certain embodiments, the compounds of Formula (VIII) are represented by Formula (VIIIa) (wherein the R groups are as described above for Formula (VIII)):

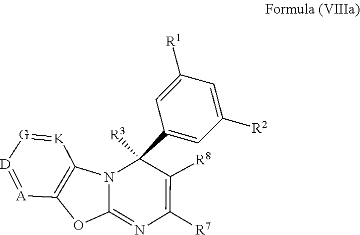

or a pharmaceutically acceptable salt thereof.

In certain embodiments, the compounds of Formula (VIII) are represented by Formula (VIIIb) (wherein the R groups are as described above for Formula (VIII)):

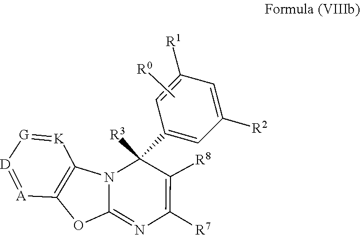

or a pharmaceutically acceptable salt thereof.

In certain embodiments of any of the foregoing or following, R⁰, R¹, and R², are each independently selected from —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy.

In certain embodiments of any of the foregoing or following, R⁰ is selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, halogen, hydroxyl, nitro, nitrile, —OR₁₁, substituted or unsubstituted C₁-C₆ alkyl, or substituted or unsubstituted C₁-C₆ haloalkyl, substituted or unsubstituted C₁-C₆ haloalkoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each independently selected from —H, methoxy, fluoro, chloro, bromo, hydroxyl, nitro, nitrile, methyl, or trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R¹ and R² are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected from methyl, ethyl, propyl, isopropyl, cyclopropyl, or cyclobutyl. In certain embodiments, any of the foregoing, may be optionally substituted.

In certain embodiments, in of any of the foregoing or following, R³ is isopropyl. In certain embodiments, isopropyl may be optionally substituted.

In certain embodiments of any of the foregoing or following, R⁷ and R⁸ are each independently selected from —H, —NR¹⁰R¹², —C(O)NR¹⁰R¹², —N(R¹²)C(O)R¹¹, nitrile, methyl, ethyl, isopropyl, cyclopropryl, cyclobutyl, cyclopentyl, cyclohexyl, phenyl, or benzyl. In certain embodiments, any of the foregoing except —H, may be optionally substituted.

In certain embodiments of any of the foregoing or following, R⁷ is methyl or phenyl. In certain embodiments, any of the foregoing may be optionally substituted.

In certain embodiments of any of the foregoing or following, R⁸ is nitrile or —C(O)NH₂. In certain embodiments, —C(O)NH₂ may be optionally substituted.

In certain embodiments of any of the foregoing or following, each occurrence of R¹¹ is independently selected from substituted or unsubstituted C₁-C₆ alkyl or substituted or unsubstituted cycloalkyl. In certain embodiments, there is no occurrence of R¹¹.

In certain embodiments of any of the foregoing or following, each occurrence of R¹⁰ and R¹² is each independently selected from —H, substituted or unsubstituted C₁-C₆ alkyl, or substituted or unsubstituted cycloalkyl, such as from —H, or substituted or unsubstituted C₁-C₆ alkyl. In certain embodiments, each occurrence of R¹⁰ and R¹² is —H. In certain embodiments, there is no occurrence of R¹⁰ and/or R¹².

In certain embodiments of any of the foregoing or following, R⁰ is independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl substituted or unsubstituted arylalkyl, substituted or unsubstituted heteroarylalkyl, substituted or unsubstituted C₁-C₆ haloalkyl, or substituted or unsubstituted C₁-C₆ haloalkoxy; R¹ and R² are each independently selected from —H, methoxy, fluoro, chloro, bromo, hydroxyl, nitro, nitrile, methyl, or trifluoromethyl;

R³ is selected from methyl, ethyl, propyl, isopropyl, cyclopropyl, or cyclobutyl;

R⁷ and R⁸ are each independently selected from H, —NH₂, —C(O)NH₂, nitrile, methyl, ethyl, isopropyl, cyclopropryl, cyclobutyl, cyclopentyl, cyclohexyl, phenyl, or benzyl; and

A, D, G and K is each independently selected from N or CR¹⁵, wherein each occurrence of R¹⁵ is independently selected from —H, halogen, hydroxyl, nitro, nitrile, substituted or unsubstituted C₁-C₆ alkoxy, substituted or unsubstituted C₁-C₆ alkyl, or substituted or unsubstituted C₁-C₆ haloalkyl, substituted or unsubstituted C₁-C₆ haloalkoxy.

In certain embodiments of any of the foregoing or following, R⁰ is selected from —H, hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹²;

R¹ and R² are each trifluoromethyl;

R³ is isopropyl;

R⁷ is —NH₂, methyl or phenyl;

R⁸ is nitrile or —C(O)NH₂; and

A, D, G and K is each independently selected from N or CR¹⁵, wherein each occurrence of R¹⁵ is independently selected from —H, halogen, hydroxyl, nitro, nitrile, substituted or unsubstituted C₁-C₆ alkoxy, substituted or unsubstituted C₁-C₆ alkyl, or substituted or unsubstituted C₁-C₆ haloalkyl, substituted or unsubstituted C₁-C₆ haloalkoxy.

In certain embodiments, the compound is selected from:

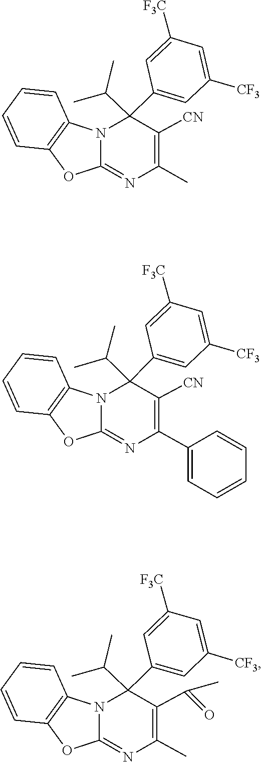 [SMILES: CC1=C(C#N)C(c2cc(C(F)(F)F)cc(C(F)(F)F)c2)(C(C)C)N2C(=N1)Oc1ccccc12 ; CC(C)C1(c2cc(C(F)(F)F)cc(C(F)(F)F)c2)C(C#N)=C(c2ccccc2)N=C2Oc3ccccc3N21 ; CC(=O)C1=C(C)N=C2Oc3ccccc3N2C1(c1cc(C(F)(F)F)cc(C(F)(F)F)c1)C(C)C]

or a pharmaceutically acceptable salt thereof.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and R² are not —H.

In one aspect, an SHMT inhibitor is a compound represented by Formula (IX):

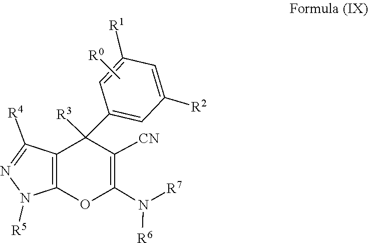

or a pharmaceutically acceptable salt thereof, wherein:

R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R³ is selected from —H, halogen, hydroxyl, nitro, nitrile, —SOR¹¹, —S(O)₂R¹¹, —S(O)₂NR¹⁰R¹², —OR¹¹, —OC(O)R¹², —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy;

R⁴ is selected from H, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl;

R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, substituted or unsubstituted heteroaryl, substituted or unsubstituted arylalkyl, or substituted or unsubstituted heteroarylalkyl, or R⁵ is selected from any of the foregoing and R⁶ and R⁷ taken together with the nitrogen atom to which they are attached form a substituted or unsubstituted 3-6 membered ring;

each occurrence of R¹¹ is independently selected from substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl; and each occurrence of R¹⁰ and R¹² is each independently selected from —H, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In some embodiments, the compound of Formula (IX) is represented by Formula (IXa) (wherein the R groups are as described above for Formula (IX)):

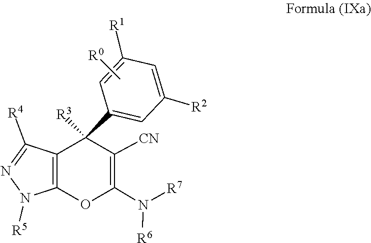

or a pharmaceutically acceptable salt thereof.

In certain embodiments, the compound of Formula (IX) is represented by Formula (IXb) (wherein the R groups are as described above for Formula (IX)):

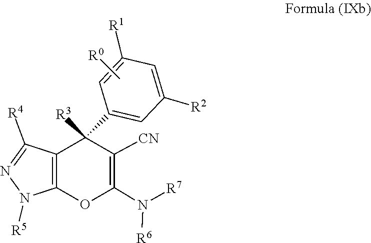

or a pharmaceutically acceptable salt thereof.

In some embodiments, of any of the foregoing or following, R⁰, R¹ and R² are each independently selected from —H, halogen, hydroxyl, nitro, nitrile, —SOR¹¹, —S(O)₂R¹¹, —S(O)₂NR¹⁰R¹², —OR¹¹, —C(O)OR¹², —C(O)R¹¹, —C(O)NR¹⁰R¹², —NR¹⁰R¹², —N(R¹²)C(O)R¹¹, —NS(O)₂R¹², substituted or unsubstituted C₁-C₆ alkyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted C₁-C₆ haloalkyl, or substituted or unsubstituted C₁-C₆ haloalkoxy.

In some embodiments, of any of the foregoing or following, R⁰ is selected from hydroxyl, —S(O)₂R¹¹, —S(O)₂NR¹⁰R¹², —OR¹¹, —C(O)NR¹⁰R¹², —NR¹⁰R¹², —N(R¹²)C(O)R¹¹, or —NS(O)₂R¹². In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In some embodiments, of any of the foregoing or following, R¹ and R² are each independently selected from —H, halogen, hydroxyl, nitro, nitrile, —OR¹¹, substituted or unsubstituted C₁-C₆ alkyl, or substituted or unsubstituted C₁-C₆ haloalkyl, substituted or unsubstituted C₁-C₆ haloalkoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In some embodiments, of any of the foregoing or following, R¹ and R² are each independently selected from —H, methoxy, fluoro, chloro, bromo, hydroxyl, nitro, nitrile, methyl, trifluoromethyl, or trifluoromethoxy. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In some embodiments, of any of the foregoing or following, R¹ and R² are each independently selected from H, methoxy, chloro, nitro, nitrile, or trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In some embodiments, of any of the foregoing or following, R¹ and R² are each trifluoromethyl. In other embodiments, R⁰ is selected from —H, halogen, substituted or unsubstituted C₁-C₆ haloalkyl, substituted or unsubstituted C₁-C₆ haloalkoxy, or substituted or unsubstituted C₁-C₆ alkyl. In other embodiments R⁰ is —H.

In certain embodiments of any of the foregoing or following, R³ is selected from —H, halogen, hydroxyl, nitro, nitrile, substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In certain embodiments of any of the foregoing or following, R³ is selected substituted or unsubstituted C₁-C₆ alkyl, substituted or unsubstituted cycloalkyl, substituted or unsubstituted heterocyclyl, substituted or unsubstituted aryl, or substituted or unsubstituted heteroaryl.

In some embodiments of any of the foregoing or following, R³ is selected from methyl, ethyl, propyl, isopropyl, cyclopropyl, or cyclobutyl. In certain embodiments, any of the foregoing may be optionally substituted.

In some embodiments of any of the foregoing or following, R³ is selected from isopropyl, cyclopropyl, or cyclobutyl. In certain embodiments, any of the foregoing may be optionally substituted.

In some embodiments of any of the foregoing or following, R³ is cyclobutyl. In certain embodiments, cyclobutyl may be optionally substituted.

In certain embodiments of any of the foregoing or following, R⁴ is selected from —H, substituted or unsubstituted C₁-C₆ alkyl, substituted or unsubstituted cycloalkyl, substituted or unsubstituted aryl, substituted or unsubstituted heteroaryl, or substituted or unsubstituted arylalkyl.

In certain embodiments of any of the foregoing or following, R⁴ is selected from —H, substituted or unsubstituted C₁-C₆ alkyl, substituted or unsubstituted cycloalkyl, substituted or unsubstituted aryl, or substituted or unsubstituted arylalkyl.

In some embodiments of any of the foregoing or following, R⁴ is selected from methyl, ethyl, isopropyl, cyclopropyl, cyclobutyl, cyclopentyl, cyclohexyl, phenyl or benzyl. In certain embodiments, any of the foregoing may be optionally substituted.

In some embodiments of any of the foregoing or following, R⁴ is methyl or isopropyl. In certain embodiments, any of the foregoing may be optionally substituted.

In some embodiments, of any of the foregoing or following, R⁴ is methyl. In certain embodiments, methyl may be optionally substituted.

In certain embodiments of any of the foregoing or following, R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, substituted or unsubstituted arylalkyl, or R⁵ is selected from any of the foregoing and R⁶ and R⁷ taken together with the nitrogen atom to which they are attached form a substituted or unsubstituted 3-6 membered ring.

In certain embodiments of any of the foregoing or following, R⁵, R⁶ and R⁷ are each independently selected from —H, —C(O)R¹¹, substituted or unsubstituted C₁-C₆ alkyl, substituted or unsubstituted cycloalkyl, substituted or unsubstituted aryl, or substituted or unsubstituted arylalkyl.

In some embodiments of any of the foregoing or following, R⁵, R⁶ and R⁷ are each independently selected from —H, methyl, ethyl, isopropyl, cyclopropyl, cyclobutyl, cyclopentyl, cyclohexyl, phenyl, benzyl, or COCH₃. In certain embodiments, any of the foregoing, except —H, may be optionally substituted.

In some embodiments of any of the foregoing or following, R⁵, R⁶ and R⁷ are each independently selected from H, methyl, phenyl, or —COCH₃. In certain embodiments, any of the foregoing, except —H, may be optionally substituted.

In some embodiments of any of the foregoing or following, R⁵ and R⁶ are each independent selected from H, methyl or phenyl. In certain embodiments, any of the foregoing, except —H, may be optionally substituted.

In some embodiments of any of the foregoing or following, R⁷ is H.

In some embodiments of any of the foregoing or following, the compound is selected from:

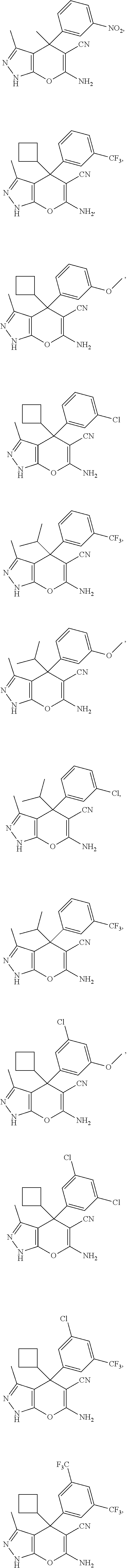

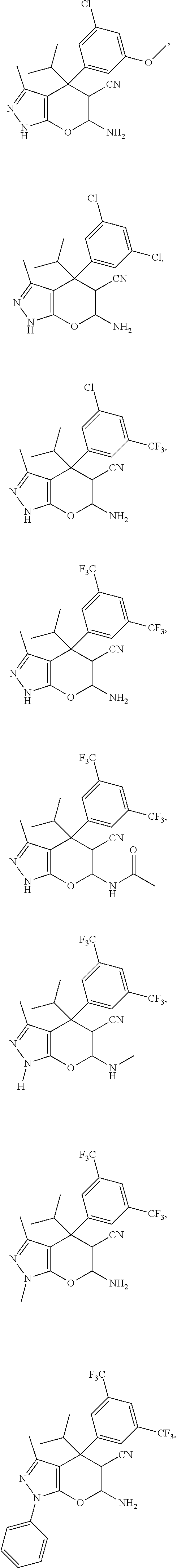

or a pharmaceutically acceptable salt of any of the foregoing.

In certain embodiments of any of the foregoing or following, the R⁰, R¹ and R² are not all simultaneously —H. In other embodiments, R⁰ is —H and the ring to which it is attached, is substituted with a single substituent (other than —H) at one of R¹ or R². In certain embodiments, R⁰ is —H, and R¹ and R² are not —H. In other embodiments, R¹ is —H and the ring to which it is attached is substituted with a single substituent (other than —H) at one of R⁰ or R². In certain embodiments, R¹ is —H, and R⁰ and R² are not —H. In other embodiments, R² is —H and the ring to which it is attached is substituted with a single substituent (other than —H) at one of R⁰ or R¹. In certain embodiments, R² is —H, and R⁰ and R¹ are not —H. In other embodiments, R⁰, R¹ and Rare not —H.

The disclosure also provides variants of Formulae (IV), (V), (VI), (VII), (VIII), and (IX), wherein

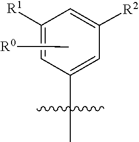

contains 1 to 3 heteroatoms (e.g., 1, 2 or 3 heteroatoms) independently selected from O or N.

In some embodiments, the SHMT inhibitor is selected from a compound in Table 2, or a pharmaceutically acceptable salt thereof.

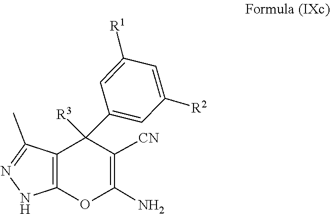

TABLE 2

| | | | | SHMT2 | SHMT1 |
|---|---|---|---|---|---|
| | R³ | R² | R¹ | IC₅₀ (nM) | IC₅₀ (nM |
| GD-07 | -CH₃ | —NO₂ | —H | >5000 | |
| HK-1 | -cyclobutane | —CF₃ | —H | >5000 | |
| HK-2 | -cyclobutane | —OMe | —H | >5000 | >5000 |
| HK-3 | -cyclobutane | —Cl | —H | 1191 | |
| HK-4 | -cyclobutane | —CF₃ | —H | 1768 | |
| HK-5 | —CH(CH₃)₂ | —CF₃ | —H | 756 | |
| HK-6 | —CH(CH₃)₂ | —OMe | —H | 4580 | |
| HK-7 | —CH(CH₃)₂ | —Cl | —H | 400.5 | |
| HK-8 | —CH(CH₃)₂ | —CF₃ | —H | 400.5 | |
| HK-9 | -cyclobutane | —Cl | —OMe | 3464 | |
| HK-10 | -cyclobutane | —Cl | —Cl | 1193 | 593 |
| HK-11 | -cyclobutane | —Cl | —CF₃ | 100 | |
| HK-12 | -cyclobutane | —CF₃ | —CF₃ | 168 | 205.7 |
| HK-13 | —CH(CH₃)₂ | —Cl | —OMe | 500 | |
| HK-14 | —CH(CH₃)₂ | —Cl | —Cl | 131 | 67 |
| HK-15 | —CH(CH₃)₂ | —Cl | —CF₃ | 35 | 20 |
| HK-16 (racemic | —CH(CH₃)₂ | —CF₃ | —CF₃ | 27 | 21 |
| mxture) | | | | | |
| HK-16 (PK-1) | —CH(CH₃)₂ | —CF₃ | —CF₃ | >5000 | >5000 |
| HK-16 (PK-2) | —CH(CH₃)₂ | —CF₃ | —CF₃ | 15 | 5 |
| HK-X1 | —CH(CH₃)₂ | —CF₃ | 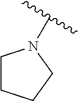 [SMILES: *N1CCCC1] | 66 | 13 |
| HK-X2 (SHIN1) | —CH(CH₃)₂ | —CH₂OH | —C₆H₅ (phenyl) | 13 | 5 |
| HK-X3 | —CH(CH₃)₂ | —CH₂OH | 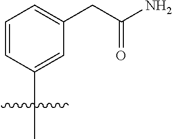 [SMILES: *c1cccc(CC(N)=O)c1] | 7.8 | 16.9 |
| Compound a | —CH(CH₃)₂ | —CF₃ | —Br | 42 | |

### Compositions, Combinations and Kits

In some embodiments, an active agent described herein (e.g., an inhibitor of serine catabolism, such as an SHMT inhibitor) is formulated with one or more pharmaceutically acceptable carriers, excipients and/or diluents. The disclosure provides such compositions, including pharmaceutical compositions. The compositions can be used in the methods described herein, e.g., to supply an agent described herein.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The characteristics of the carrier will depend on the route of administration.

Agents or pharmaceutical compositions may be administered to cells in vitro, such as by addition to culture media. Additionally or alternatively, agents or pharmaceutical compositions may be administered by a route of administration, such as oral, parenteral, intravenous, intra-arterial, cutaneous, subcutaneous, intramuscular, topical, intracranial, intraorbital, ophthalmic, intravitreal, intraventricular, intracapsular, intraspinal, intracisternal, intraperitoneal, intranasal, aerosol, central nervous system (CNS) administration, or administration by suppository. In some embodiments, an agent or pharmaceutical composition is administered topically, systemically, or locally. For example, agents and compositions may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. Agents or compositions may also be formulated as part of an implant or device, or formulated for slow or extended release. When administered parenterally, e.g., by intravenous, cutaneous or subcutaneous injection, agents and compositions are preferably in a pyrogen-free, physiologically acceptable form.

A preferred pharmaceutical composition for intravenous, cutaneous, or subcutaneous injection should contain, in addition to the toxicity-reducing compounds, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the compound of the present disclosure may also contain stabilizers, preservatives, buffers, antioxidants, or other additives known to those of skill in the art. Techniques and formulations generally may be found in Remington's Pharmaceutical Sciences, Meade Publishing Co., Easton, Pa.

A composition may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Agent(s) or compositions may be in the form of a tablet, capsule, powder, solution or elixir, for example, for oral administration. When administered in tablet form, the pharmaceutical composition may additionally contain a solid carrier such as a gelatin or an adjuvant. The tablet, capsule, and powder may contain from about 5% to about 95% by weight of an active agent, and preferably from about 10% to about 90% by weight of the active agent. When administered in liquid form, a liquid carrier such as water, petroleum, oils of animal or plant origin such as peanut oil, mineral oils, phospholipids, tweens, triglycerides, including medium chain triglycerides, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition typically contains from about 0.5% to about 90% by weight of an active agent, and preferably from about 1% to about 50% by weight of the active agent.

In solid dosage forms for oral administration (capsules, tablets, pills, dragees, powders, granules, and the like), a composition may contain one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active agent, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

The pharmaceutical compositions may be in the form of a liposome or micelles in which the toxicity-reducing compounds are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

Suspensions, in addition to the active agent may contain suspending agents, such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The amount of an active agent in a pharmaceutical composition will depend upon, for example, the nature and severity of the condition being treated, the agent used, and on the nature of prior treatments the patient has undergone. Ultimately, the practitioner will decide the amount of the active agent to use to treat each individual patient. Representative doses of the present disclosure include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. It is contemplated that the various pharmaceutical compositions used to practice the methods of the present disclosure should contain, in some embodiments, about 0.1 μg to about 100 mg (preferably about 0.1 mg to about 50 mg, more preferably, about 1 mg to about 2 mg) of active agent per kg body weight.

The desired dose may conveniently be presented in a single dose or as divided doses administered at appropriate intervals, for example, as 2, 3, 4 or more sub-doses, per day. The sub-dose itself may be further divided, e.g., into a number of discrete loosely spaced administrations. The daily dose can be divided, especially when relatively large amounts are administered as deemed appropriate, into several, for example, 2, 3 or 4 administrations. If appropriate, depending on individual behavior, it may be necessary to deviate upward or downward from the daily dose indicated.

In some embodiments, compositions can include more than one active agent, e.g., a combination. Accordingly, also provided herein is a combination comprising an inhibitor of serine catabolism (e.g., an SHMT inhibitor) and an additional therapeutic agent (e.g., an electron transport chain inhibitor). In some embodiments, a combination further includes a pharmaceutically acceptable carrier, excipient or diluent.

When two active agents are administered in combination, they can be administered in separate formulations. Accordingly, also provided herein is a kit comprising an inhibitor of serine catabolism (e.g., an SHMT inhibitor), or a composition comprising the inhibitor of serine catabolism, and an additional therapeutic agent(s) (e.g., an electron transport chain inhibitor), or a composition comprising the additional therapeutic agent(s). In some embodiments, the kit further comprises written instructions for administering the inhibitor of serine catabolism and the additional therapeutic agent(s) to a subject to treat a disorder described herein.

### Exemplification

Serine Catabolism Feeds NADH when Respiration is Impaired

Summary: NADH provides electrons for aerobic ATP production. In cells deprived of oxygen or with impaired electron transport chain activity, NADH accumulation can be toxic. To minimize such toxicity, elevated NADH inhibits the classical NADH producing pathways: glucose, glutamine, and fat oxidation. Here and in Yang et al., 2020, Cell Metab. 31, 809-821, the entire contents of which are incorporated herein by reference, through deuterium tracing studies in cultured cells and mice, it is shown that folate-dependent serine catabolism also produces substantial NADH. Strikingly, when respiration is impaired, serine catabolism through methylene tetrahydrofolate dehydrogenase (MTHFD2) becomes a major NADH source. In cells whose respiration is slowed by hypoxia, metformin, or genetic lesions, mitochondrial serine catabolism inhibition partially normalizes NADH levels and facilitates cell growth. In mice with engineered mitochondrial complex I deficiency (NDUSF4−/−), serine's contribution to NADH is elevated and progression of spasticity is modestly slowed by pharmacological blockade of serine degradation. Thus, when respiration is impaired, serine catabolism contributes to toxic NADH accumulation.

Mammals break down carbohydrates, amino acids, and fatty acids for energy generation (Chen et al., 2018; Hui et al., 2017; Kalucka et al., 2018; Lundsgaard et al., 2018; Neinast et al., 2019). Through TCA turning in mitochondria, these nutrients are burned into CO₂ while producing reducing power in the form of NADH. Electrons from NADH are then transferred to mitochondrial complex I and downstream components of the electron transport chain (ETC), eventually reducing oxygen to water (Titov et al., 2016). By inducing a proton gradient, these reactions power mitochondrial ATP synthase, which provides the bulk of ATP in mammals.

The most common physiological impediment to respiration is hypoxia. Respiration impairment can also be induced by genetic lesions of the ETC (e.g., in mitochondrial disorders) and by pharmaceuticals. Metformin, the first-line treatment for type 2 diabetes, acts at least in part as a complex I inhibitor (El-Mir et al., 2000; Fullerton et al., 2013; Hunter et al., 2018; Liu et al., 2016b; Madiraju et al., 2014; Madiraju et al., 2018; Owen et al., 2000; Wheaton et al., 2014). The consequences of ETC inhibition may vary depending on the respiratory chain component involved. For example, the reduction of complexes III and IV is increased by hypoxia but not by complex I inhibition (Chandel et al., 1998; Wheaton et al., 2014).

Increased NADH and decreased ATP are direct biochemical consequences of ETC impairment. An important question is their relative importance. In cell culture, increasing glycolysis can compensate for impaired mitochondrial ATP generation (DeBerardinis et al., 2008; Lunt and Vander Heiden, 2011). To avoid NADH accumulation, however, requires an alternative electron acceptor, such as pyruvate. Major consequences of NADH accumulation include blockade of TCA turning, impairment de novo aspartate synthesis, and resulting decreases in protein and nucleotide synthesis (Birsoy et al., 2015; Gameiro et al., 2013; Mullen et al., 2012; Sullivan et al., 2015; Wise et al., 2011; Yoo et al., 2008). Cell growth can be rescued by engineered aspartate assimilation or by expression of LbNOX, an enzyme that oxidizes NADH without generating ATP (Sullivan et al., 2018; Titov et al., 2016). Thus, a major driver of the adverse consequences of ETC impairment is NADH accumulation.

Even though ETC impairment is growth inhibitory, for unclear reasons, genetic ETC lesions have been shown to activate the kinase mTORC1. This induces expression of a wide range of anabolic genes, with the one-carbon (1C) metabolic enzyme methylenetetrahydrofolate dehydrogenase 2 (MTHFD2) strongly upregulated (Ben-Sahra et al., 2016; Khan et al., 2017; Nikkanen et al., 2016; Tyynismaa et al., 2010). In mitochondria, MTHFD2 oxidizes 5,10-methylenetetrahydrofolate (THF) to generate the purine precursor 10-formyl-THF, while reducing NAD to NADH. The mTORC1 inhibitor rapamycin decreases MTHFD2 expression, normalizes 1C metabolism, and prolongs the survival of mice with engineered complex I deficiency (NDUSF4−/− mice) (Johnson et al., 2013; Khan et al., 2017; Zheng et al., 2016). The functional importance of MTHFD2 in this context remains, however, unexplored. More generally, the reactions producing the accumulated NADH when ETC activity is impaired remain understudied. Here, using deuterium labeled tracers, it is discovered that mitochondrial serine catabolism can produce substantial NADH, especially in pancreas, spleen and tumors. This pathway turns out to be the major source of NADH for cells with impaired ETC activity due to hypoxia, pharmacological ETC inhibition, or genetic ETC lesions. In these contexts, inhibition of mitochondrial serine catabolism improved metabolic homeostasis and enhanced growth of cultured cells. In addition, it slowed onset of spasticity in complex I deficient mice.

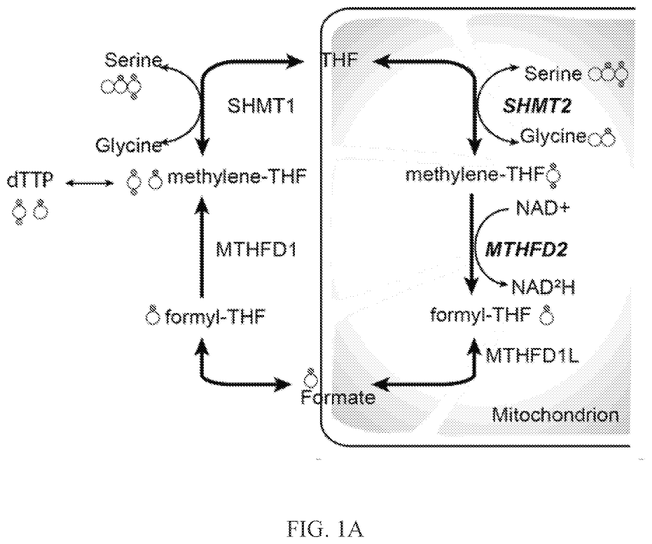

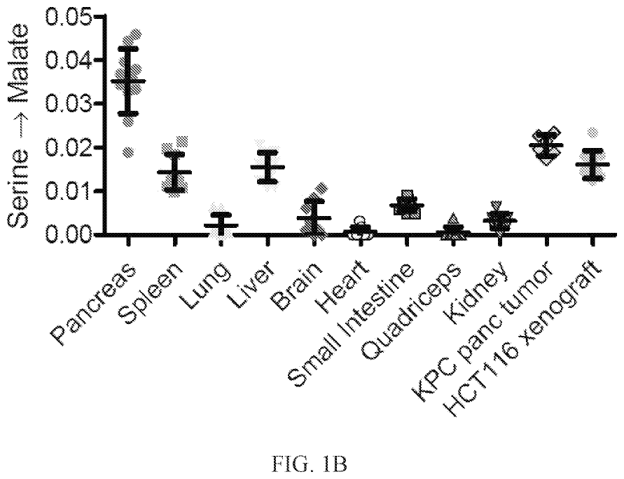

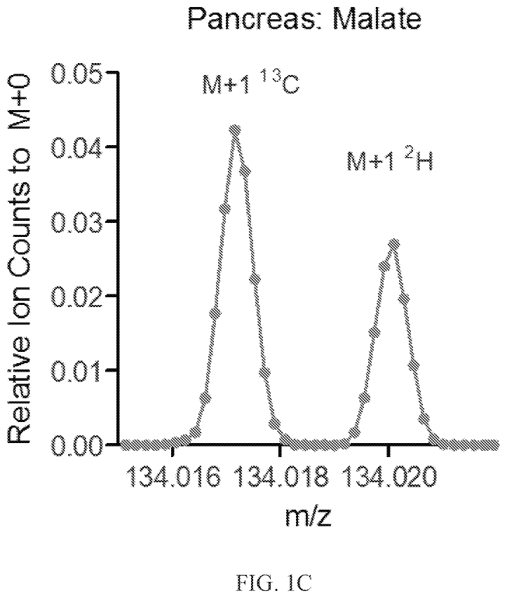

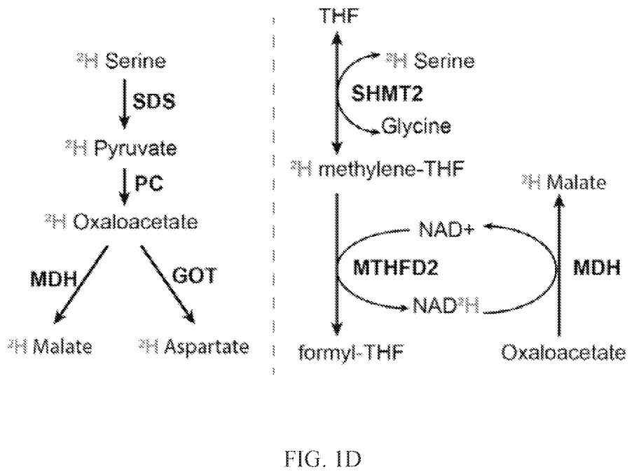

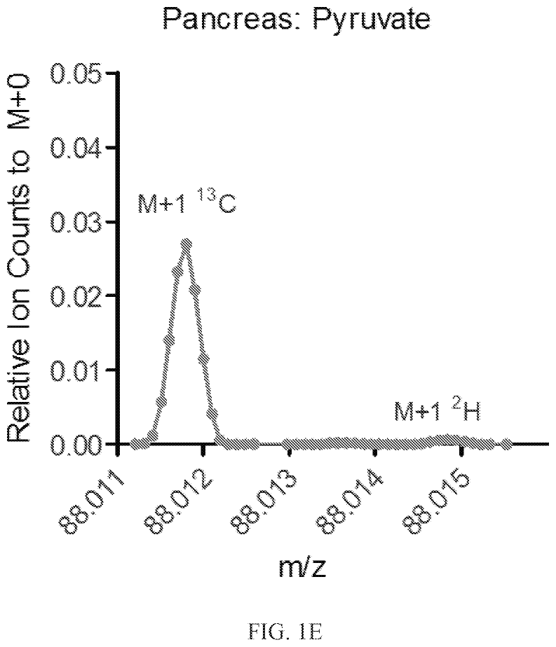

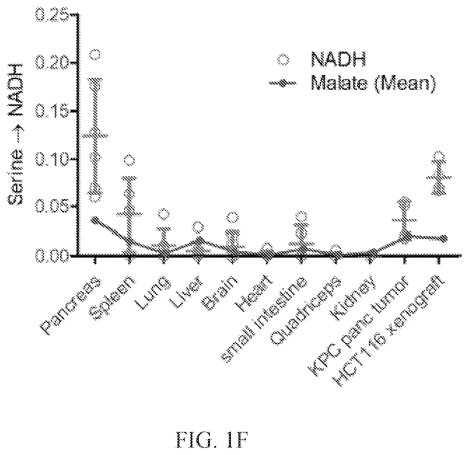

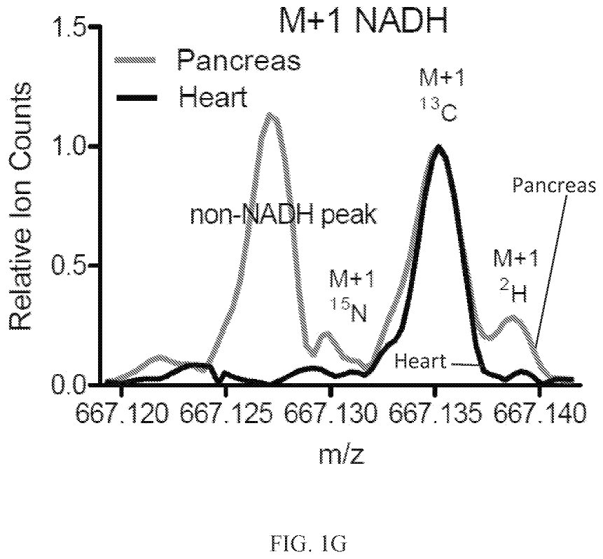

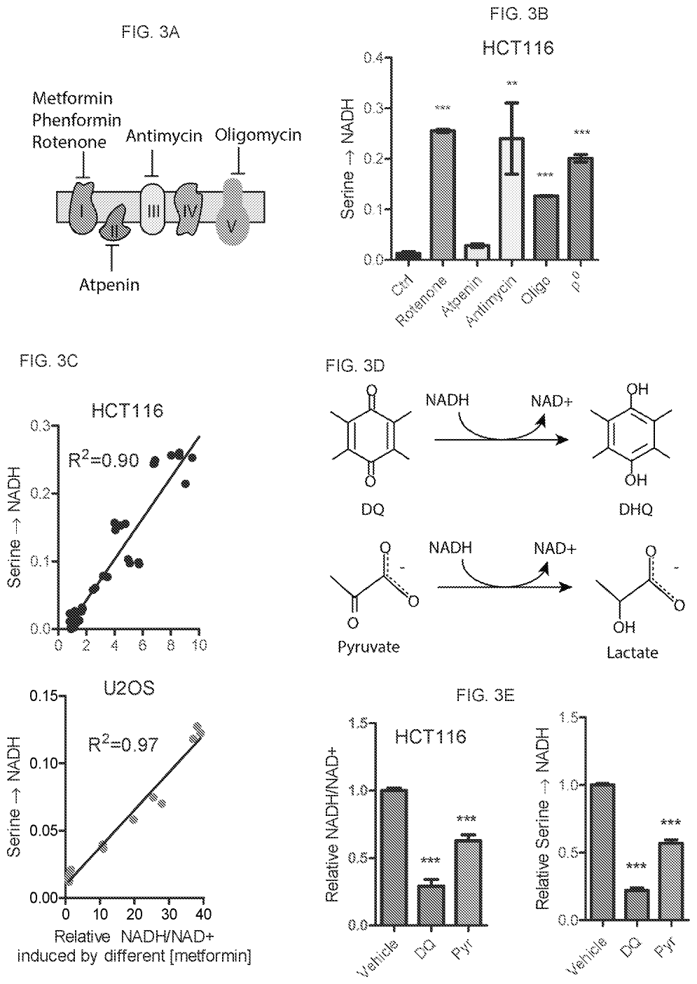

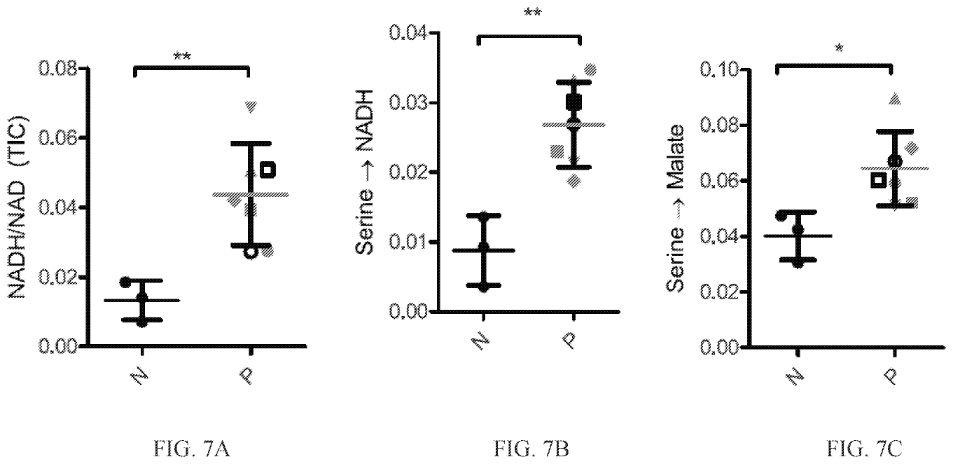

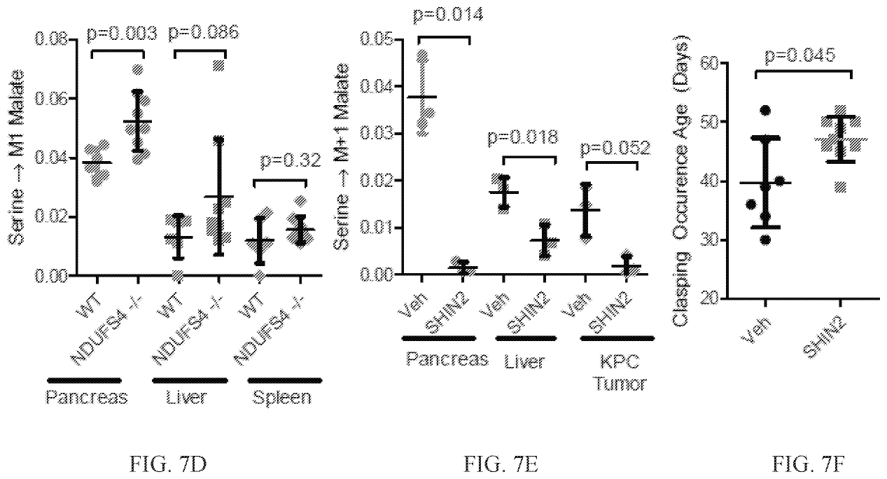

The studies described herein and in Yang et al. (2020) are based on setting up a tracing strategy to directly label active hydride in NADH by using deuterium labeled nutrients (serine, glutamine, lactate, fatty acids). The present disclosure is based in part on the discovery that a significant fraction of mitochondrial NADH is derived from serine in both in vitro cell culture and in vivo tissue/tumor system (FIGS. 1-3). Its contribution linearly correlates with cellular redox state, indicated from NADH/NAD+(FIG. 3C). With an orderly drop of NADH generation from glucose and glutamine, serine catabolism becomes the primary source when cellular respiration is arrested with ETC inhibitors or in hypoxia. Serine catabolism, paradoxically, becomes toxic since loss of serine catabolism rescues cell growth in (pseudo) hypoxia (FIGS. 3-6). Finally, the studies described herein and in Yang et al. (2020) show that the pancreas from NDUFS4−/− mice and fibroblasts derived from Leigh Syndrome patients also increase serine as NADH hydride donor, which suggests a novel therapeutic window to treat mitochondrial diseases (FIG. 7).

Methods: The following materials and methods were used in the experiments described herein.

| Reagent | Source | Catalog No. |
|---|---|---|
| [2,3,3-²H]Serine | Cambridge Isotope | DLM-582 |
| [U- [symbol] C]Glucose | Cambridge Isotope | U-13C6 |
| [U- [symbol] C]Glutamine | Cambridge Isotope | CLM-1822 |
| [U- [symbol] C]Palmitate | Cambridge Isotope | CLM-6059 |
| [2,3,3,4,4-²H]Glutamine | Cambridge Isotope | DLM-1826 |
| [3,3,3-²H]Lactate | Cambridge Isotope | DLM-9071 |
| [U-31-²H]Palmitate | Sigma Aldrich | 366897 |
| Metformin | Sigma Aldrich | PHR 1084 |
| Phenformin | Sigma Aldrich | PHR1573 |
| Atepnin | Cayman chemicals | 119509-24-9 |
| Rotenone | Sigma Aldrich | 557363 |
| Oligomycin | Sgma Aldrich | 75351 |
| Antimycin | Sigma Aldrich | AB674 |
| Duroquinone | Sigma Aldrich | D223204 |
| UK5099 | Cayman Chemical | 16900 |
| IACS-10759 | Axon Medchem | 2909 |
| Mouse: C57BL/6 | Charles River Laboratories | 027 |
| Mouse: NDUFS4−/+ | lackson Laboratory | 027058 |
| Mouse: CD1 nude | Charles River Laboratories | 086 |
| KrasLSL · G12D/+ × | Gift from Haiyong Han (TGEN) | |
| p53R172H/+ × PdxCretg/+ | | |
| pancreatic tumor | | |
| Naive CD8a+ T cell isolation kit | Miltenyi Bictec | 130-096-543 |
| Pan T cell solation kit II | Miltenyi Blotec | 130-095-130 |
| LS columns | Miltenyi Blotec | 130-042-401 |
| Pre-seperation filters (70 μm) | Miltenyi Blotec | 130-095-823 |
| Murine IL-2 | Peprotech | 212-12 |
| Anti-mouse CD3 | SioXCell | Clone 145-2C11, BE0001-1 |
| Anti-mouse CD28 | BloXCell | Clone 37051, BE0001-1 |
| Seahorse Mitostress Kit | Agilent | 103015-100 |
| CyQUANT NIF cell proliferation | Thermofisher | C35006 |
| assay | | |
| CyQUANT cell proliferation for | Thermofisher | C7026 |
| seahorse assay | | |
| Malate dehydrogenase | Sigma Aldrich | M1567 |
| PDH | Sigma Aldrich | P7032 |
| KGDH | sigma Aldrich | K1502 |
| MTHFD2 | Gift from Raze Therapeutics | |
| NAD | Sigma Aldrich | N0632 |
| NADH | Sigma Aldrich | N8129 |
| 5,10-meTHF | Schircks Laboratories | 16.226 |
| CoA | Sigma Aldrich | A2056 |

[symbol]

indicates data missing or illegible when filed

Cell culture. Cancer cell lines were incubated with DMEM+10% FBS. HCT116, HepG2, Panc1, A549, Jurkat, 293T, C2C12 were purchased from ATCC, HAP1 were purchased from Horizon Discovery, U2OS is kindly provided by Reuben Shaw (Salk Institute).

Isolation, culture and stimulation of naïve CD8+ or Total T cells. To isolate naïve CD8+T or total T cells, spleens were harvested and single cell suspensions prepared by manual disruption and passage through a 70 m cell strainer in PBS supplemented with 0.5% BSA and 2 mM EDTA. After red blood cell lysis, naïve CD8+T or total T cells were purified by magnetic bead separation using commercially available kits following vendor instructions (Naïve CD8a+ T Cell Isolation Kit, mouse or Pan T Cell Isolation Kit II, mouse, Miltenyi Biotec Inc).

Cells were cultured in complete RPMI media (11875-093, supplemented with 10% FBS, 100 U/ml penicillin, 100 μg/ml streptomycin, and 50 μM 2-mercaptoethanol). For activation, T cells were stimulated for 48 h with plate-bound anti-CD3 (10 μg/ml) and anti-CD28 (5 μg/ml) in complete media supplemented with 100 μM alanine and recombinant IL-2 (100 U/mL). Cells were maintained in complete RPMI media supplemented with 100 U/mL recombinant IL-2. Metabolomics experiments were performed at day 4-5 post-activation.

Mouse breeding and isotope tracing. Animal studies followed protocols approved by the Princeton University Institutional Animal Care and Use Committee and the Rutgers University Institutional Animal Care and Use Committee. NDUFS4+/− mice were purchased from Jackson Laboratory. The mice were on normal light cycle (7 AM-7 PM). In vivo infusion was performed on 9-week old normal C57BL/6 mice pre-catheterized on the right jugular vein, and 4-week old NDUFS4−/− or NDUFS4+/+ mice pre-catheterized on the jugular vein. Infusion was performed for 2.5 h to achieve isotopic pseudo-steady state. The mouse infusion setup included a tether and swivel system, connecting to the button pre-implanted under the back skin of mice. Mice were fasted from 9:00 am to 2 pm, and infused from 2 pm till 4:30 pm. 400 mM [2,3,3-2H]serine was dissolved in saline, and infused via the catheter at a constant rate (0.1 l/min/g mouse weight) using a Just infusion Syringe Pump. At the end of infusion, mice were dissected and tissues were clamped in aluminum foil and stored in liquid nitrogen.

NDUFS4−/− mice were housed with normal mice and gel food was supplied to guarantee sufficient food supply. Mice were grouped as vehicle control group and SHIN2 treatment groups. SHIN2 was dissolved in 20% 2-hydroxypropyl-cyclodextrin solution, and delivered by intraperitoneal (50 μg/g mice) b.i.d. Mice weights were recorded every day. Limb clasps were recorded daily, with mice annotated as having the pathological limb clasping phenotype on the third consecutive day of clasping. 20% weight loss of the peak weight for each mouse is considered as the humane endpoint.

Mouse Xenograft and allograft. For tumor xenograft tracing experiments, 6- to 7-week-old male CD1/nude mice were injected in the rear flanks with HCT-116 (1×106 cells in 100 μL 1:1 PBS:Matrigel). For tumor allograft tracing experiment, 6- to 8-wk-old male C57BL6 mice were injected in the rear flank with KrasLSL.G12D/+x p53R172H/+x PdxCretg/+ pancreatic tumor tissue suspension (200 μL 1:1 DMEM:Matrigel). Jugular vein catheterization surgery was performed when tumor size reached 250 mm3 [volume=1/2 (length×width×height)]. Animals were euthanized when tumors reached 1,000 mm3 or if they displayed any signs of distress or morbidity. Tracing experiments were performed after 1 week of post-surgery monitoring. Tumors were removed and immediately clamped to aluminum foil using Wollenberger clamp and stored in liquid nitrogen. For Long term storage, tissues were kept in −80° C. until future sample preparation.

Metabolite Extraction from Cell Culture:

Adherent cells. Cells were seeded in 6 well plates overnight and then were treated with ETC inhibitors. Serine-free DMEM with 10% dialyzed FBS was supplemented with 400 M [2,3,3, 2H]serine. Medium was replaced by the isotope tracing medium with corresponding drugs, and cells were incubated for 3 h. Medium was aspirated, and metabolism was quenched with extraction buffer (40:40:20 acetonitrile:methanol: water with 0.5% formic acid; 40 μl of extraction solvent per 1 μl packed cell volume)(Lu et al., 2018). Plates were placed on dry ice for 10 mins, and thereafter the extraction solution was neutralized with 15% NH4HCO3 (70 μl for 800 μl extraction buffer). Cells were then scraped off from plates by using a cell lifter (Corning 3008), transferred into Eppendorf tubes and centrifuged in a benchtop microfuge at maximum speed for 30 min at 4° C. Supernatant was transferred to LC-MS vials for analysis.

T cells. RPMI-1640 media without glucose, glycine or serine was supplemented with 11 mM glucose, 0.133 mM glycine, 0.286 mM [2,3,3-2H]serine, 10% dFBS, 100 U/ml penicillin, 100 μg/ml streptomycin, 50 μM 2-mercaptoethanol and 100 U/mL recombinant IL-2. Cells were seeded at 106 cells/mL and incubated for 3 hours. They were then transferred to 1.5 mL Eppendorf tubes and pelleted (30 s, 6000 g, RT). Media was removed by aspiration and metabolome extraction was performed by the addition of 75 μL of extraction buffer. After a 5-min incubation on ice, the extraction solution was neutralized by the addition of NH4HCO3 as above. After centrifugation (15 min, benchtop microfuge maximum speed, 4° C.), the clean supernatant was transferred to LC-MS vial for analysis.

Metabolites extraction from tissue, tumors and serum. Tissues and tumors were collected from mice and immediately clamped into liquid nitrogen using Wollenberger clamp, tissue were stored in −80° C. Frozen tissues were transferred into 2 ml Eppendorf tubes, which are precooled on dry ice, and pulverized by using cyromill. The resulting tissue powder was weighed (around 10 mg) and mixed well by vortexing in extraction buffer (40 μL extraction buffer per mg tissue). The extraction solution was neutralized with NH4HCO3 as above and centrifuged in a microfuge at maximum speed for 30 min at 4° C. Supernatant was transferred to LC-MS vials for analysis.

Blood samples were drawn from mice tail, using microvette CB300 Z, and kept on ice. After centrifugation (10 min, benchtop microfuge maximum speed, 4° C.), serum were collected to 1.5 ml tube and store in −80° C. for future analysis. 5 μl of serum were mixed with 200 μl of extraction buffer (40:40:20 acetonitrile: methanol: water with 0.5% formic acid) and neutralized with 15% NH4HCO3. After centrifugation (30 min, benchtop microfuge maximum speed, 4° C.), supernatant were transferred into LC-MS vial for analysis.

LC-MS. LC separation was achieved using a Vanquish UHPLC system (Thermo Fisher Scientific) and an Xbridge BEH Amide column (150×2 mm, 2.5 m particle size; Waters, Milford, Mass.), column temperature 25° C. Solvent A is 95:5 water: acetonitrile with 20 mM ammonium acetate and 20 mM ammonium hydroxide at pH 9.4, and solvent B is acetonitrile. The gradient was 0 min, 85% B; 2 min, 85% B; 3 min, 60% B; 9 min, 60% B; 9.5 min, 35% B; 13 min, 5% B; 15.5 min, 5% B; 16 min, 85% B; 20 min, stop run. Injection volume was 15 μL. The Q-Exactive Plus mass spectrometer was operated in negative ion mode scanning from m/z 70-1000 with a resolution (at m/z 200) of 140,000 (AGC target 3e6, Maximum IT 200 ms) (Wang et al., 2019). A second scan from m/z 640-765 with a resolution (at m/z 200) of 35,000 (AGC target 5e5, maximum IT 200 ms) was used to measure NAD(P), NAD(P)H, and their labeling. The purpose of the second scan was to enhance sensitivity for NAD(P)(H) by restricting the range of ions in the orbitrap and to maximize the accuracy of isotope ratios by decreasing the scan range and mass resolution (Su et al., 2017).

Natural isotope abundance correction: Accucor was used to correct all of data except NADH from mice (FIG. 1E) for natural isotope abundance (Su et al., 2017). For NADH data from mice, it was necessary to correct also for the interfering peak (non-NADH peak in FIG. 1F). For this purpose, data were corrected using Elemcor, using unlabeled tissue data as the background (Du et al., 2019; Millard et al., 2012). The background correction approach is necessary because orbitrap (Q-Exactive, resolution 78K at 660 m/z) cannot resolve the 2H-NADH peak neatly from either the 13C-NADH peak or the non-NADH interfering peak.

Calculation of 2H-serine contribution to malate, aspartate, and NADH: For malate and aspartate, the 2H-labeling fraction was measured directly (fractional size of 2H-peak relative to sum of all peaks, after isotope correction as above). For NADH, the 2H-labeling fraction was measured based on the active hydride labeling (x), which was determined from the labeling pattern of NAD+ and NADH by least square fitting in MATLAB of the observed experimental data (after natural isotope correction) to the below vectors (Liu et al., 2016a).

$$NAD = ( \begin{matrix}{a}_{0} \\ {a}_{1} \\ {a}_{2}\end{matrix} ) \qquad NADH = ( \begin{matrix}{a}_{0} ( 1 - x ) \\ {a}_{1} ( 1 - x ) + {a}_{0} x \\ {a}_{2} ( 1 - x ) + {a}_{1} x \\ {a}_{2} x\end{matrix} )$$

For cell culture experiments, serine was nearly completely labeled and fractional labeling as above is reported in the manuscript without further correction. For the in vivo experiments, measured labeling fractions were normalized to the fractional labeling of serine in the tissue of interest. Serine labeling is measured as the fraction M+2 or M+3 serine, as both M+2 and M+3 serine (from [2,3,3-2H]serine) are labeled at the hydrogen that is transfer to NADH by MTHFD2.

FT-ICR MS: FT-ICR MS was used to resolve M+1 2H NADH from M+1 13C NADH (only for FIG. 1E). Analysis was carried out using a 9.4 T Bruker SolariX FT-ICR MS (Bruker Daltonics Inc., MA, USA) with an ESI source in negative mode. Continuous accumulation of selected ions (CASI) was utilized to increase sensitivity. The CASI window was 50 mass units with a center mass of 660. The ion accumulation time was set to 60 ms.

The mass spectra were calibrated externally with 1 mM arginine solution in negative mode. Resolving power at the FT-ICR was measured to be 260 K at m/z 660, which is sufficient to isolate M+1 2H NADH from M+1 13C NADH, as well as the similar mass non-NADH peak (which we discovered via this analysis).

Seahorse XF96e. Cells were seeded to Seahorse XF96 cell seeding plates (101085-004) for overnight attachment. Metformin was added to DMEM+10% dialyzed FBS, and cells were treated for 24 h and oxygen consumption measured. Agilent mitochondrial stress kits (103015-100) were loaded in XF96 calibration plates, and used to test mitochondrial function in different conditions. Oxygen consumption rates were normalized to DNA content by using DNA qualification kits (Thermo Fisher C7206).

Enzymatic Assay for MTHFD2. NAD (Sigma N0632), NADH (Sigma N8129) and 5, 10-meTHF (Schircks Laboratories, 16.226) solution were freshly prepared each time in water. Reaction mixtures consisted of 50 mM KH2PO4 pH 7.4, 5 mM dithiothreitol (DTT), 5% DMSO, and 0.005 mg/mL MTHFD2 (obtained from Raze Therapeutics), and NAD and NADH as indicated in the Figures. Reaction mixtures were incubated for 5 min at 37° C. before initiation of the reaction by addition of 5,10-meTHF to a final concentration of 0.4 mM using multichannel. The plate was mixed for 5 s in the plate reader (Biotek Synergy HT), and the rate of change in absorbance at 340 nm due to NADH accumulation was recorded over the duration of linear NADH accumulation.

Enzymatic Assay for MDH. NAD and NADH solutions were freshly prepared in water; malate solution was prepared in 50 mM Tris-HCl and adjusted pH to 7.6 with 10 M KOH. Reaction mixtures consisted of 30 mM Tris-HCl pH 7.6, 0.00047 mg/ml malate dehydrogenase (Sigma M1567), and NAD and NADH as indicated in the Figures. Reaction mixtures were incubated for 5 min at 25° C. before initiation of the reaction by addition of malate to a final concentration of 20 mM using multichannel. The plate was mixed for 5 s in the plate reader, and absorbance at 340 nm recorded as above.

Enzymatic assay for PDH and KGDH: Pyruvate dehydrogenase and alpha-ketoglutarate dehydrogenase solutions were freshly prepared to a concentration 0.075 unit/ml with buffer containing 20 mM sodium phosphate and 1 mM MgCl2, pH7.2. A mixture of thiamine pyrophosphate and Coenzyme A was freshly prepared in the same buffer and diluted by 5 times before use. NAD and NADH solutions were freshly prepared in the buffer through a serial dilution; sodium pyruvate and alpha-ketoglutarate solutions were prepared in buffer in a stock 52 concentration of 100 mM. All solutions were prepared on ice. Reaction mixtures consisted of a final concentration of 0.015 unit/ml of each enzyme, 0.2 mM TPP and 0.5 mM CoA, and NAD and NADH as indicated in the Figures. Reaction mixtures were incubated for 5 min at room temperature before initiation of the reaction by addition of substrate to a final concentration of 5 mM using multichannel. The reaction took place at 37° C. The plate was mixed for 5 s in the plate reader, and absorbance at 340 nm recorded as above.

Cell Proliferation Assays. Cells were seeded in seeded in 96 well plates (Corning 3603) for overnight attachment. Medium (with or without drugs) was changed next day, and cell numbers were recorded using Thermofisher CyQUANT kit.

Blood sampling from pigs. Pig studies followed protocols approved by the University of Pennsylvania Institutional Animal Care and Use Committee. Five-month old male Yorkshire pigs were fed at 4 PM and then overnight fasted. At 7 AM in the next morning, anesthesia was initiated with a ketamine IM injection (15˜35 mg/kg), followed by intubation and maintenance of anesthesia with inhaled isoflurane at 0.5˜5%. Arterial and venous access was obtained to enable fluid infusion (NaCl 4-10 mcg/kg/min), blood pressure monitoring, 49 pharmacological control of blood pressure (0.2-2.0 mcg/kg/min dobutamine and 0.1-5.0 mcg/kg/min phenylephrine, as required), and blood sampling. The primary initiative venous access was via ultrasound-guided percutaneous puncture of the external jugular vein. Arterial access was through the neck to the carotid artery. The surgical site was blocked with Bupivacaine SQ just after the first incision. The subcutaneous tissues and muscle layers were carefully dissected, with hemostasis achieved with electrocautery. For blood sampling from the hepatic and the portal vein, a catheter was placed on each vein. Blood from inferior vena cava, internal jugular vein, femoral vein, renal vein, splenic vein, inferior pancreatic vein, inferior mesenteric vein, lateral auricular vein, and coronary sinus were obtained by drawing with a 27G needle connected to 1 mL syringe. Blood was drawn from each vessel twice at 1-2 min intervals, except for the coronary sinus (only a single draw). After completion of blood sampling, the animal was euthanized. Blood samples were placed at room temperature for 10-20 min, and centrifuged at 1,000×g for 10 min to obtain serum. Data represents the median value for triplicate technical replicates.

Mouse Model. B6.129S4-Ndufs4+/− mice were purchased from Jackson laboratories. The mice are heterozygous for a protein in mitochondrial complex 1 and have been bred to generate homozygous knockout mouse for the study of mitochondrial deficiency. These mice are a model of mitochondrial disease and knockouts are healthy until approximately 5 weeks age, but develop lethal encephalomyopathy by 7 weeks.

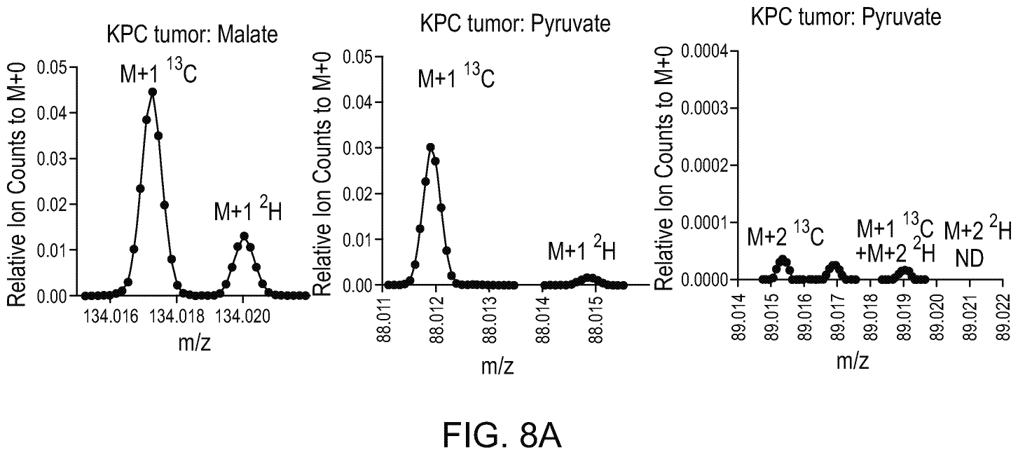

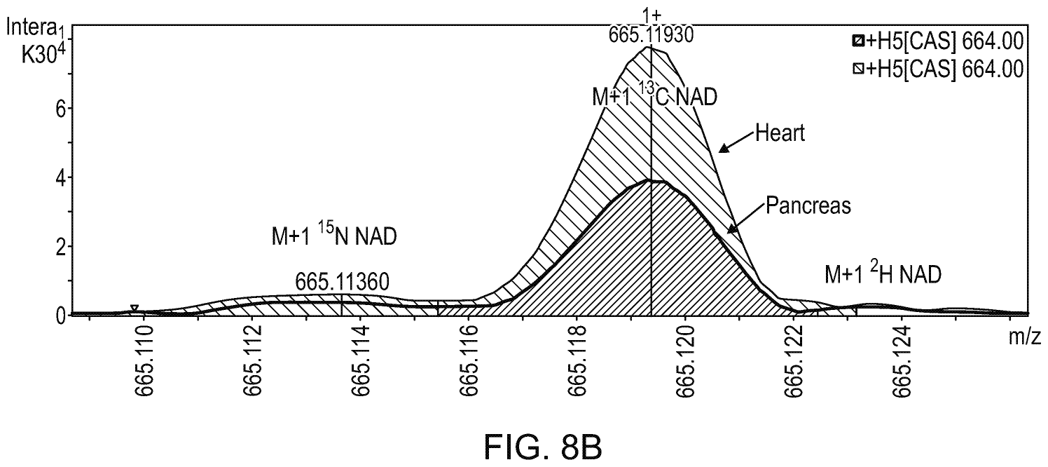

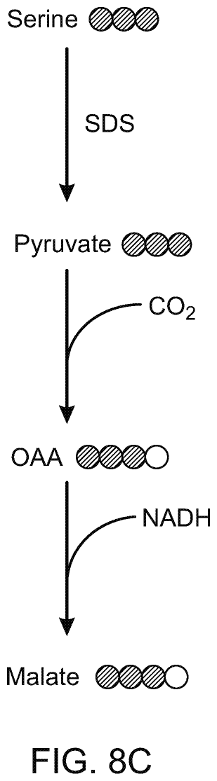

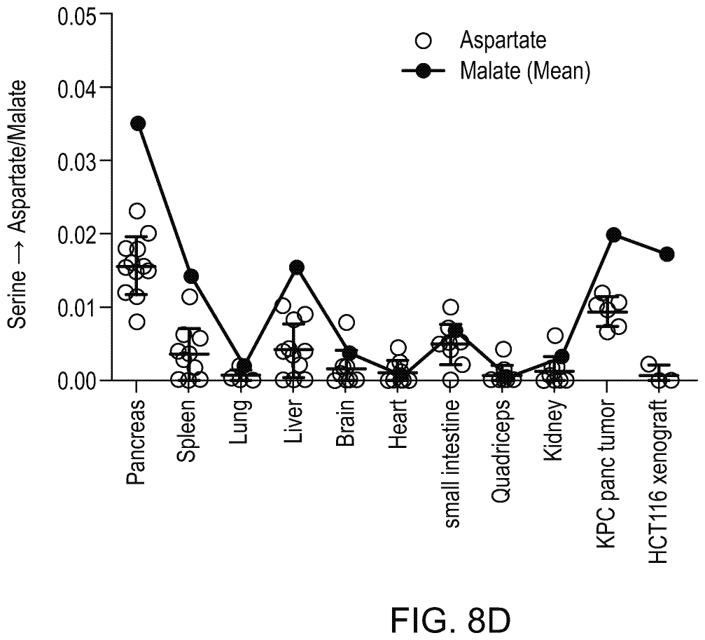

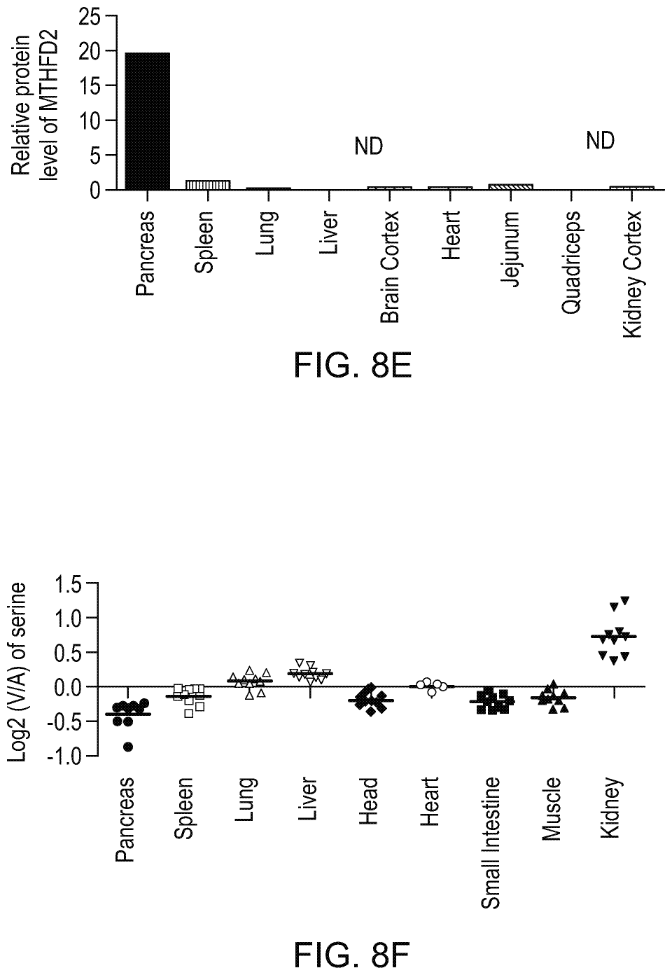

### Example 1. Serine Catabolism Feeds NADH In Vivo

[2,3,3-²H] serine is an isotope tracer useful for determining whether one-carbon (1C) units for thymidine synthesis are generated via cytosolic or mitochondrial serine catabolism (FIG. 1A). In the studies described herein, upon infusing this tracer into mice, unexpected labeling of malate was observed, which is not the canonical product of serine or 1C metabolism (FIG. 1). High-resolution mass spectrometry confirmed that the labeled peak was ²H-malate (as opposed to natural abundance ¹³C-malate) (FIGS. 1C, 8A). Reported values are normalized to circulating [2,3,3-²H] serine labeling (i.e. fraction of serum serine labeled but do not include any other correction factors. While the raw extent of malate labeling was small, due to tracer loss via H-D exchange, even modest ²H labeling is often biologically meaningful.

There are two potential pathways for ²H transfer from serine to malate: (i) via pyruvate (the ²H remains covalently bound to the serine carbon skeleton, which becomes ²H-pyruvate, ²H-oxaloacetate, and eventually ²H-malate) and (ii) via NADH made from serine through 1C metabolism (the ²H from serine is transferred via 1C metabolism to NAD²H, which reduces unlabeled oxaloacetate) (FIG. 1D). No ²H-labeling was observed in pyruvate from [2,3,3-2H] serine, ruling out this pathway (FIG. 1E, 8B). In contrast, ²H-labeling was observed in NADH (FIG. 1F), although it is cautionary to note that the in vivo measurements of NAD²H are difficult, as the mass spectrometer cannot resolve the ²H from ¹³C peaks for NADH. Because of the relatively high mass of NADH, standard orbitrap mass spectrometry cannot resolve ²H-from ¹³C-NADH. Accordingly, Fourier-transform ion cyclotron resonance mass spectrometry (FTICR-MS, 260K resolution at m/z 660) was used to directly confirm ²H-labeled NADH (FIG. 1G). Thus, folate-mediated serine catabolism generates NADH.

A distinguishing feature of the 1C-NADH pathway is transfer of ²H from serine to malate without carbon transfer. Indeed, in most organs (with exceptions being the liver and brain), ²H malate labeling from [2,3,3-²H] serine in excess of ¹³C transfer from [U-¹³C] serine was observed (FIG. 8D). The key redox enzyme of the 1C-NADH pathway is MTHFD2. Labeling of NADH and malate from ²H-serine was highest in pancreas, which also had the highest MTHFD2 gene expression (FIG. 8E) and greatest serine consumption based on depletion of serine in the draining venous blood (FIG. 8F). Substantial malate ²H-labeling was also observed in allografted genetically engineered pancreatic cancer tumors (KPC tumors) and xenografted colon cancer tumors (HCT116). Collectively, these observations suggest that serine catabolism is a meaningful NADH source in certain organs (especially pancreas, spleen, and liver) and tumors.

### Example 2. Serine Catabolism is a Major NADH Source when Respiration is Impaired

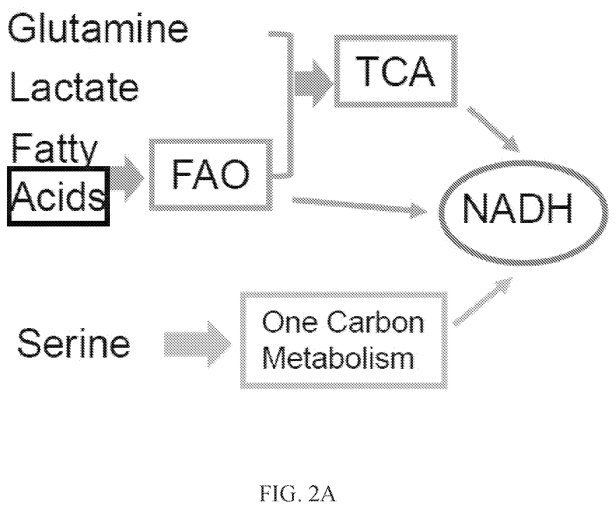

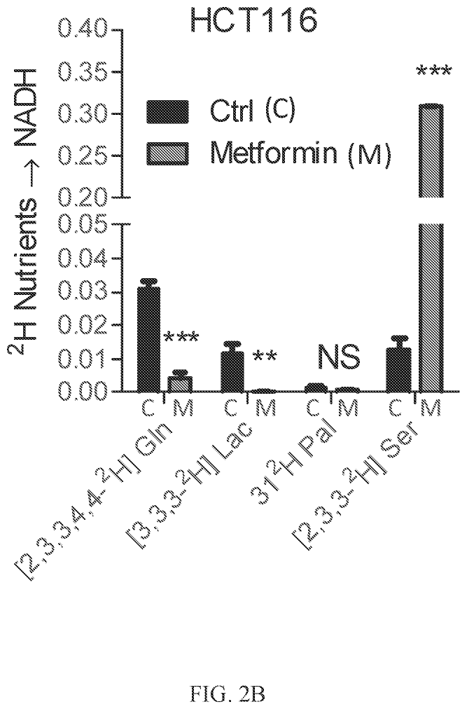

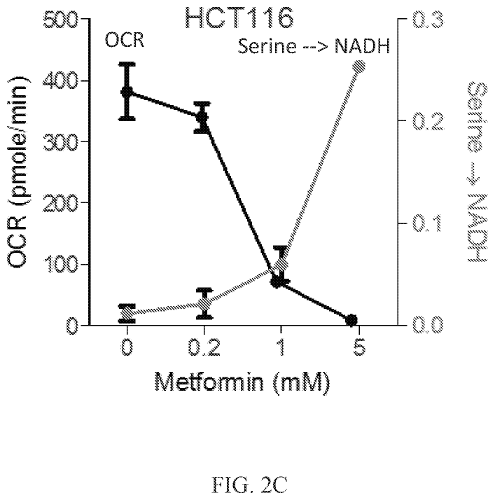

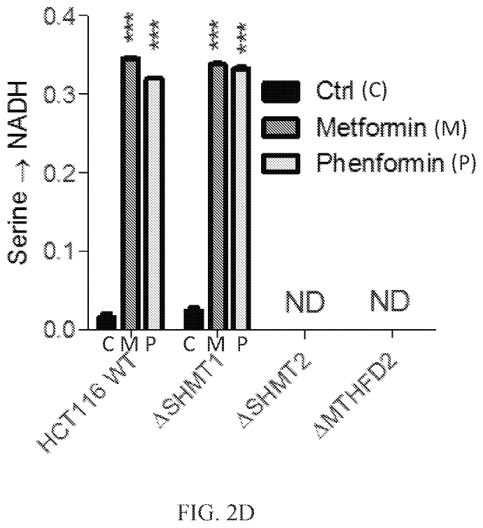

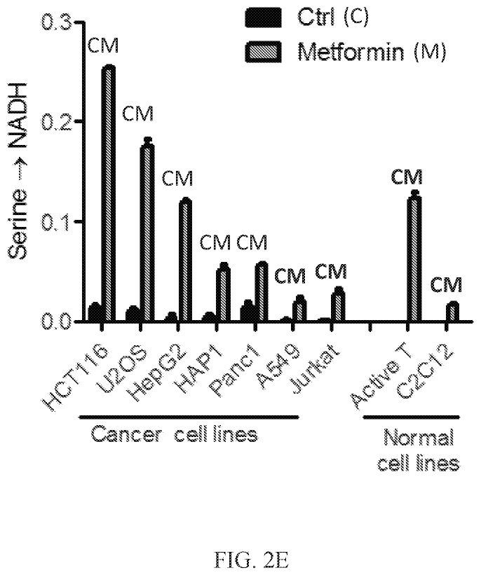

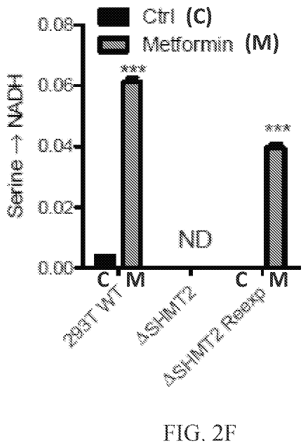

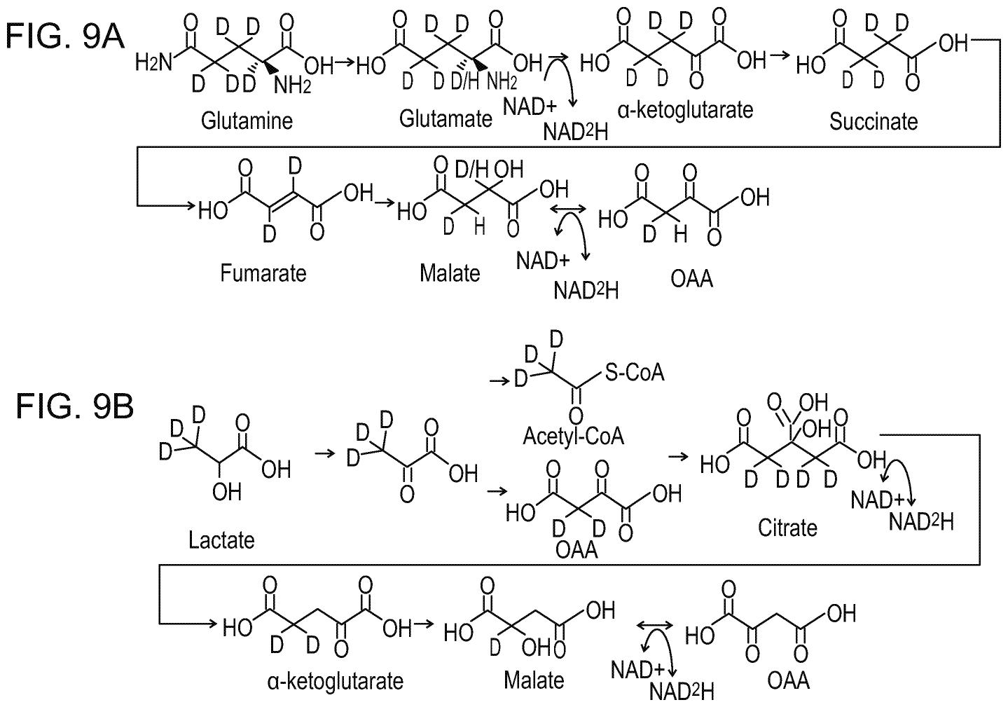

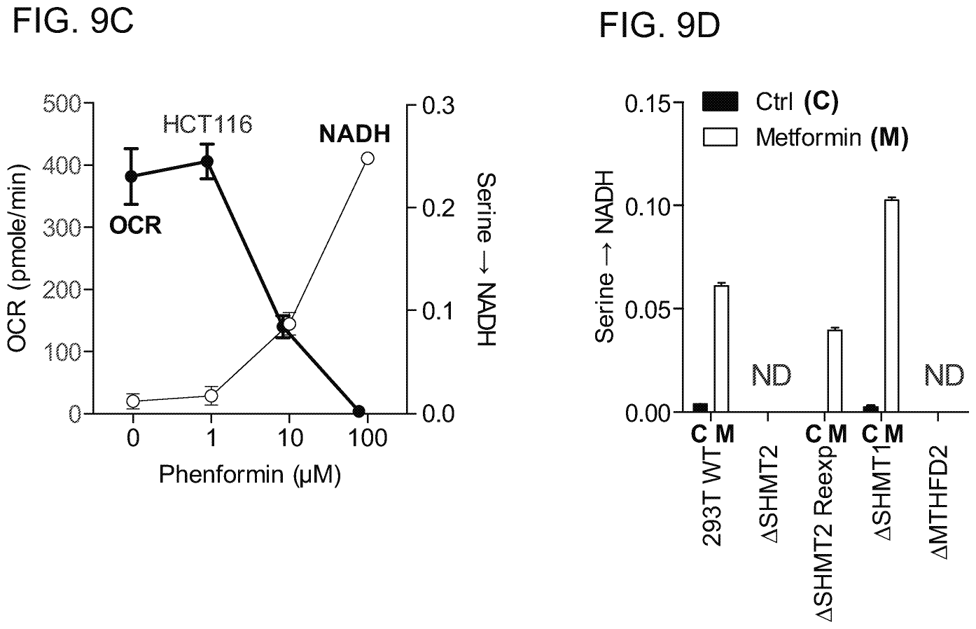

To better understand the contribution of serine to NADH production, cell culture was used to compare NADH labeling from classical major NADH sources (glutamine, lactate, fat) with that from serine (FIGS. 2A, 9A-9B). These experiments involved transferring cells from unlabeled to fully labeled media and reported values correct for natural isotope abundance but no other factors. Under standard tissue culture conditions, NAD²H from serine was less than glutamine but larger than lactate or palmitate, verifying that serine can be a substantial NADH source (FIG. 2B).

To assess how the contribution of these sources shifts when respiration is impaired, the measurements were repeated in the presence of the complex I inhibitor metformin. Strikingly, metformin (and also phenformin) dramatically elevated in the fractional contribution of serine to NADH (FIGS. 2C, 2D, 9C). This enhancement by metformin was seen across a diversity of tumor and normal cell lines (FIG. 2E). Both the basal and metformin-induced serine contribution to NADH occurred via folate-mediated mitochondrial serine catabolism, as it was fully blocked by knockout of the mitochondrial folate enzymes SHMT2 and MTHFD2 (FIG. 2F). SHMT2 re-expression rescued the serine contribution to NADH (FIG. 9D). Thus, mitochondrial serine catabolism via the SHMT2-MTHFD2 pathway is a major NADH source when respiration is impaired.

### Example 3. NADH/NAD Ratio Dictates Serine's NADH Contribution

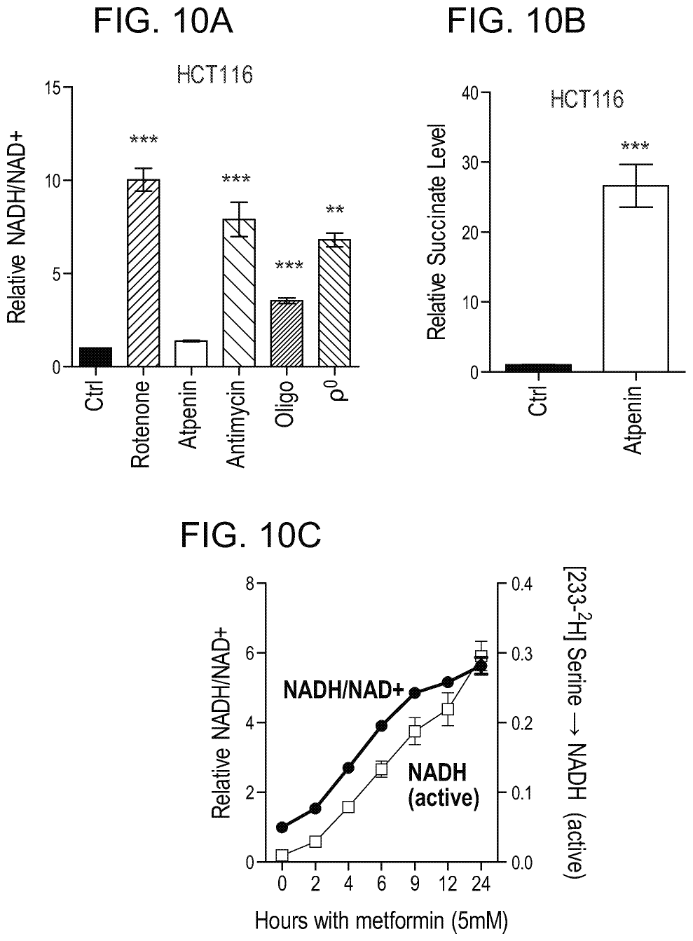

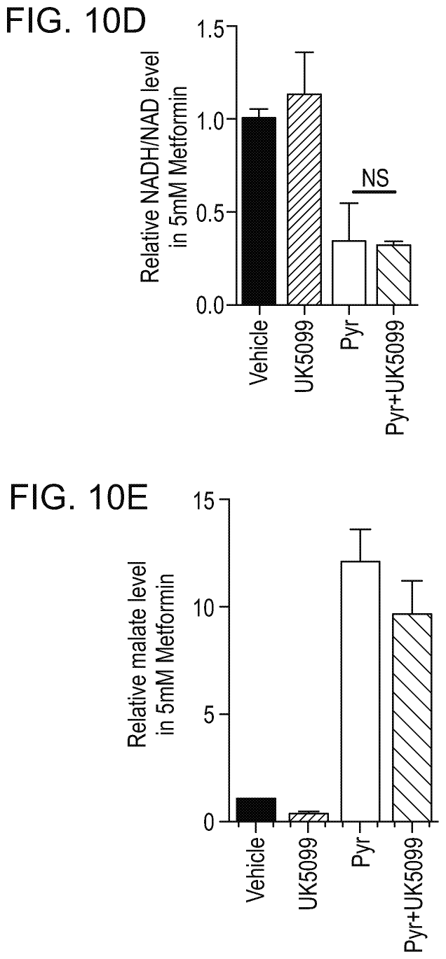

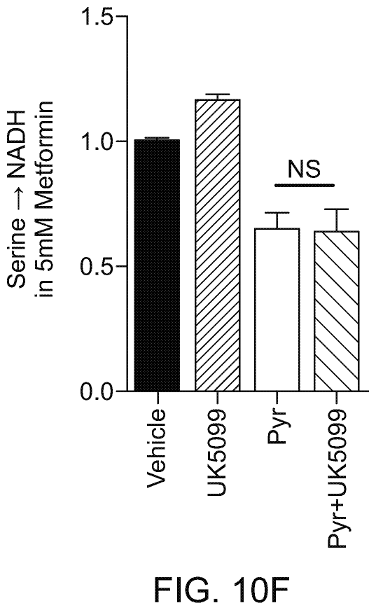

It was hypothesized that an elevated NADH/NAD ratio disfavors other routes of NADH production, and therefore respiration impairment increases the fractional serine contribution to NADH. As a first step towards exploring this hypothesis, the impact of different respiratory chain inhibitors on serine's NADH contribution was examined. Loss of mitochondrial DNA (ρ⁰ cells) or pharmacological inhibition of complexes I, III, or V increased the NADH/NAD ratio and elevated the fractional serine contribution to NADH (FIGS. 3A-B, 10A). In contrast, inhibition of complex II, which is not required for NADH oxidation and whose inhibition increased succinate but not NADH/NAD, did not (FIGS. 3A, 3B, 10B).

To further explore the connection between the intracellular NADH/NAD ratio and serine's NADH contribution, both parameters in response to different dosages of metformin treatment were measured and found to correlate linearly (FIG. 3C). A similar correlation was observed as a function of metformin exposure duration (FIG. 10C). As an orthogonal approach to manipulating NADH/NAD, duroquinone or pyruvate was added to metformin-treated cells. These compounds have been previously shown to act as electron acceptors and thereby decrease the NADH/NAD ratio (FIG. 3D). As hypothesized, their addition decreased the fractional contribution of serine to NADH (FIG. 3E). This did not appear to require pyruvate entry into mitochondria, because the effect of pyruvate was not blocked by UK5099, an inhibitor of mitochondrial pyruvate carrier. Instead, pyruvate seems to be converted into lactate in the cytosol, with normalization of the cytosolic NAD/NADH ratio indirectly impacting mitochondrial redox status (e.g., via the malate-aspartate shuttle). In sum, across a diversity of manipulations, as the NADH/NAD ratio increases, serine's fractional contribution to NADH also increases.

### Example 4. High NADH/NAD+ Shuts Off Other NADH Sources but not Serine Catabolism

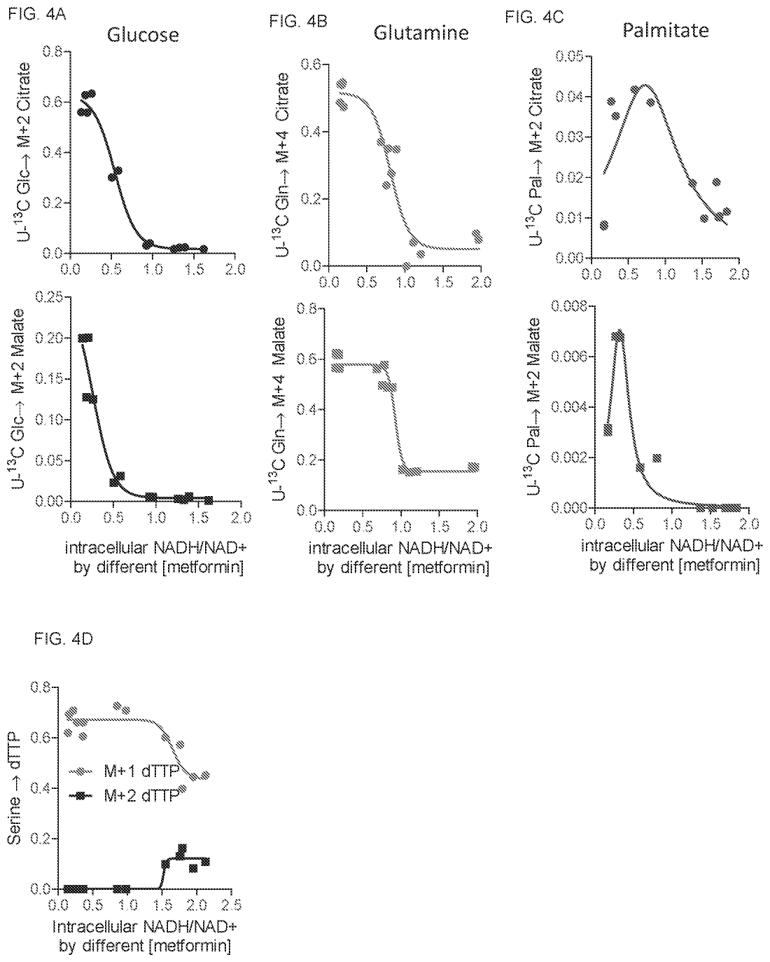

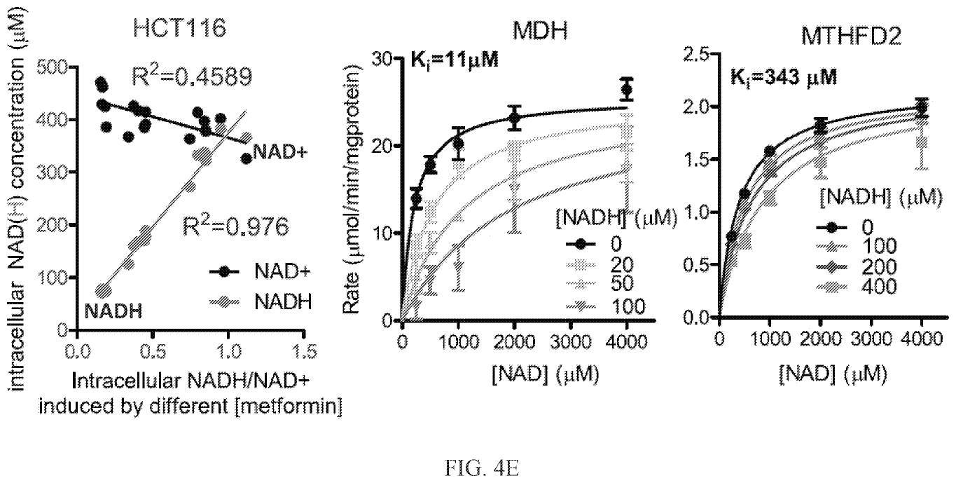

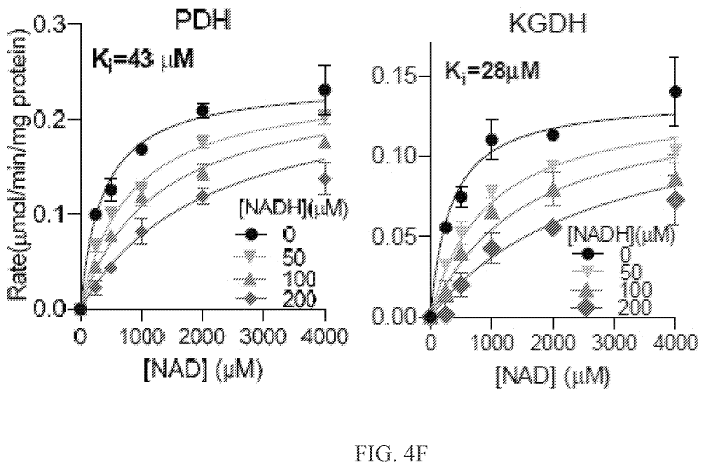

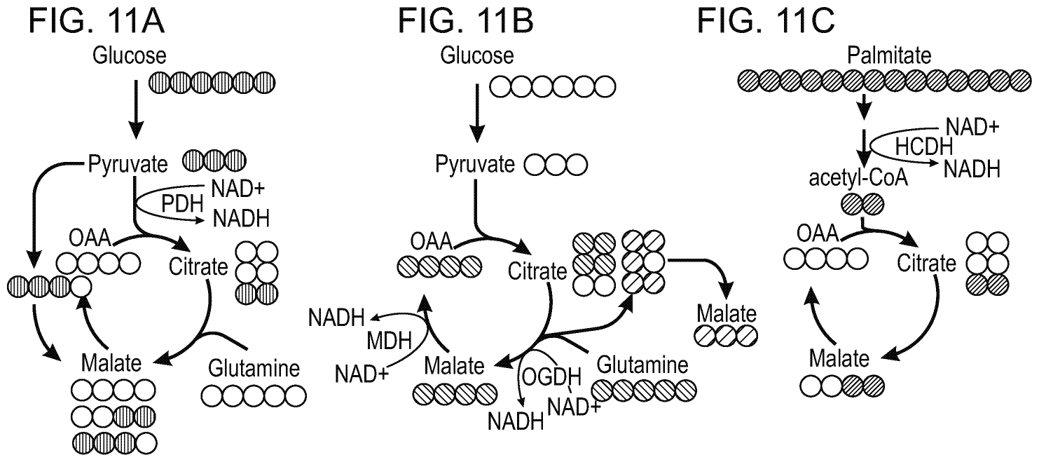

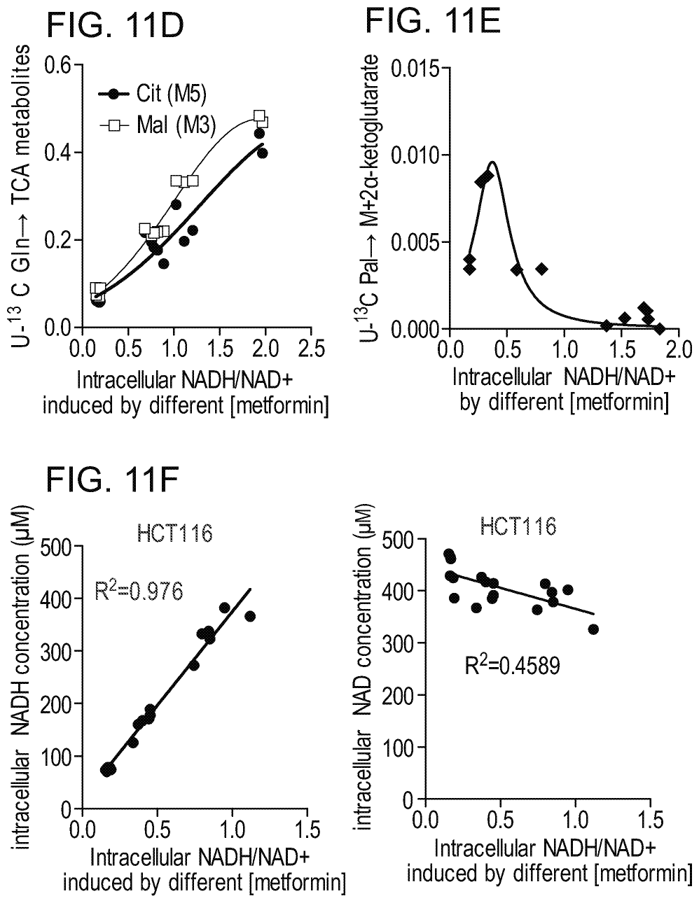

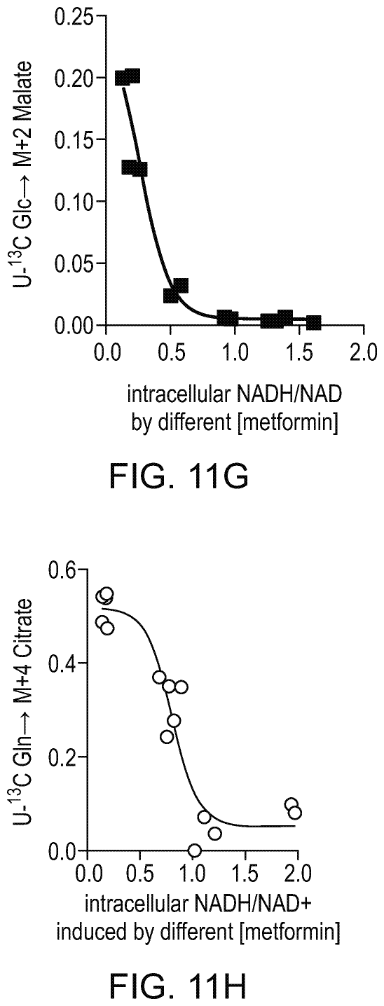

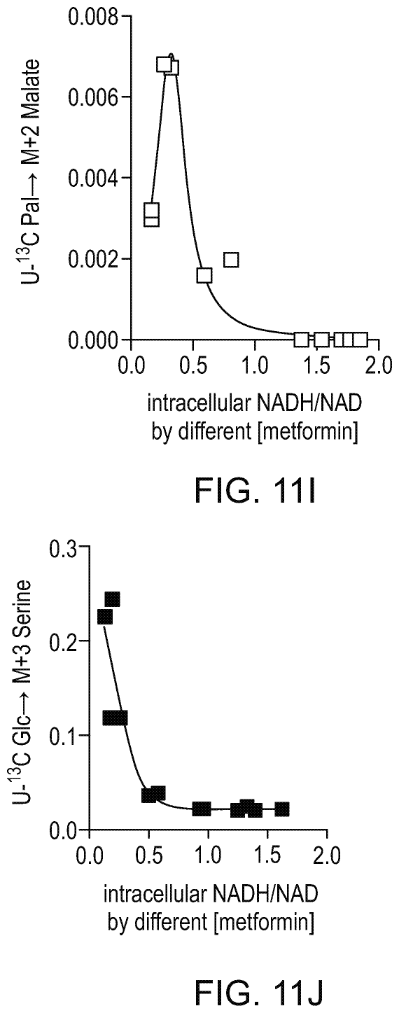

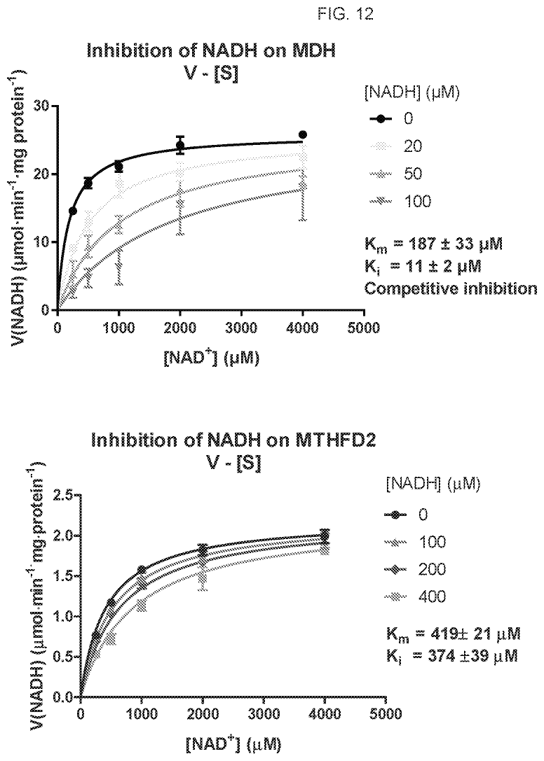

The correlation between NADH/NAD and serine's fractional NADH contribution could be explained by either (i) enhanced serine catabolism when NADH/NAD rises or (ii) shutting off of other routes of NADH production. The latter was considered more likely. To test for it, the contribution of ¹³C-glucose, glutamine, and palmitate to TCA intermediates as a function of the NADH/NAD ratio induced by respiration inhibition with metformin was measured (for pathway schematics, see FIG. 11A-C). As NADH/NAD rose, the TCA contribution from glucose was nearly eliminated. This reflected both pyruvate dehydrogenase inhibition (which was evident as loss of M+2 citrate from glucose) and blocked TCA turning, which was evident as decreased malate and citrate M+4 from glutamine (with glutamine instead contributing to the TCA via reductive carboxylation, FIGS. 4A-B, 11D). As oxidation of glucose and glutamine dropped, initially the fatty acid contribution to TCA rose, but then decreased (FIGS. 4C, 11E). Similarly, de novo serine synthesis, which produces cytoplasmic NADH, nearly ceased.

To examine whether mitochondrial serine catabolism is also shut off, thymidine labeling from [2,3,3-²H]serine was measured. Mitochondrial serine catabolism generates M+1 thymidine, whereas the alternative cytosolic pathway generates M+2 thymidine. There was no decrease in the thymidine M+1 labeling fraction up to a 5-fold increase in NADH/NAD, a level of respiration impairment sufficient to block TCA turning driven by glucose, glutamine and fat and de novo serine synthesis (FIG. 4D). Yet, higher NADH/NAD ratios did result in a partial shift towards cytosolic serine metabolism. Nevertheless, catabolism of serine is uniquely robust to rising NADH/NAD.

Intracellular NADH level can well reflect NADH/NAD (FIG. 11F). To examine the underlying biochemical mechanism, the sensitivity of pyruvate dehydrogenase (PDH), α-ketoglutarate dehydrogenase (KGDH) and malate dehydrogenase versus MTHFD2 to increasing NADH was examined. These assays were conducted in the presence of adequate NAD as substrate, which is appropriate given that physiologically large changes in the NADH/NAD ratio driven by increasing NADH is observed, with NAD largely maintained (FIG. 4E). Although all of the enzymes were subject to product inhibition by NADH, MTHFD2 was at least 10-fold less sensitive, maintaining full activity up to 200 μM NADH, a typical level observed in respiration-impaired cells (FIGS. 4F, 11F). Thus, the key redox enzyme of serine catabolism is NADH-resistant, and therefore serine catabolism persists even after other NADH production pathways shut off.

### Example 5. Loss of Mitochondrial Serine Catabolism Paradoxically Facilitates Cell Growth in Respiration-Impaired Cells

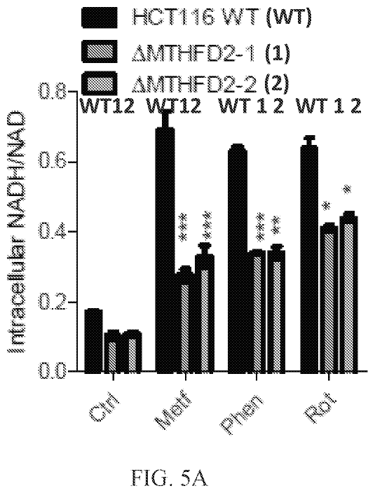

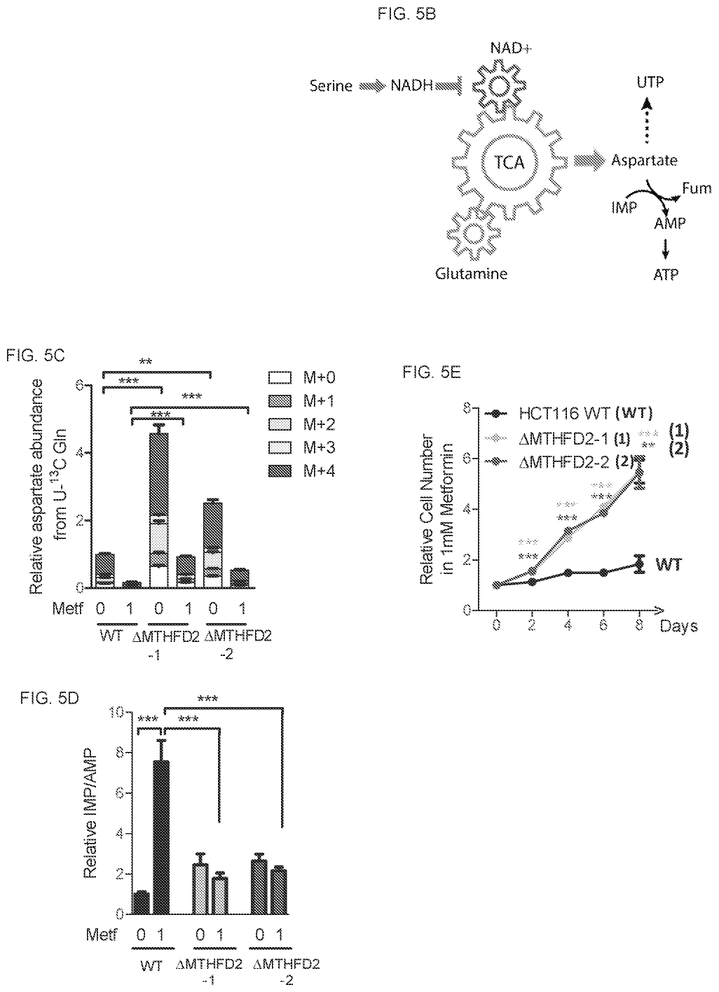

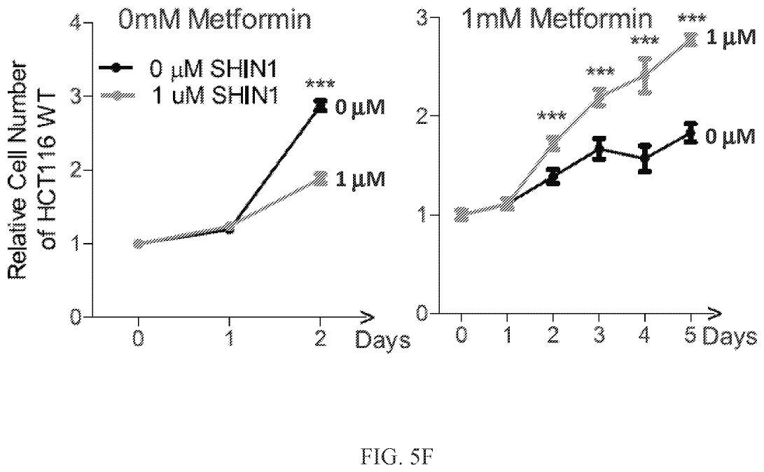

Serine catabolism is the primary cellular source of 1C units, which are required for nucleotide synthesis and therefore growth. Inhibition of 1C metabolism with anti-folates is clinically employed to treat proliferative conditions, including cancer and autoimmunity. Thus, serine catabolism is classically considered pro-growth. At the same time, however, maintenance of redox homeostasis is critical to cell growth. To examine whether persistent serine catabolism during respiration impairment might induce a pathologically high NADH/NAD ratio and thereby paradoxically impair cell growth, metabolite levels and growth of MTHFD2 knockout cells, in the presence and absence of complex I inhibitors were studied. Such cells tended to have a lower NADH/NAD ratio under basal conditions, which was significantly lower than wild-type cells after complex I inhibition with metformin, phenformin or rotenone (FIG. 5A).

In response to respiration impairment, elevated NADH/NAD tends to deplete the most oxidized intermediate of the TCA cycle, oxaloacetate, and thereby its transamination partner aspartate. Aspartate is required for protein, purine, and pyrimidine biosynthesis (FIG. 5B). Manipulations that restore aspartate can accelerate growth in respiration-impaired conditions. Increased aspartate in cells lacking MTHFD2 under both basal and metformin-treated conditions was observed (FIG. 5C). Aspartate contributes to purine synthesis by donating its amino group to IMP to make AMP. The IMP/AMP ratio is accordingly an effective measure of aspartate depletion. In the presence of metformin, unlike wild-type cells, MTHFD2 knockout cells maintained a normal IMP/AMP ratio (FIG. 5D). Thus, in response to respiration impairment, cells lacking mitochondrial serine catabolism are better at maintaining metabolic homeostasis.

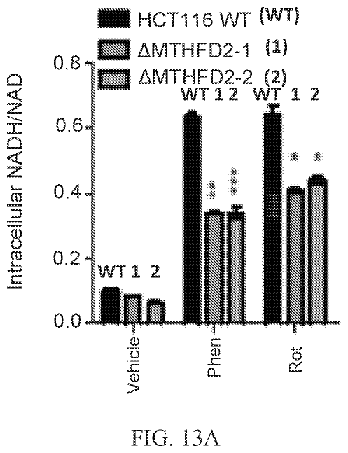

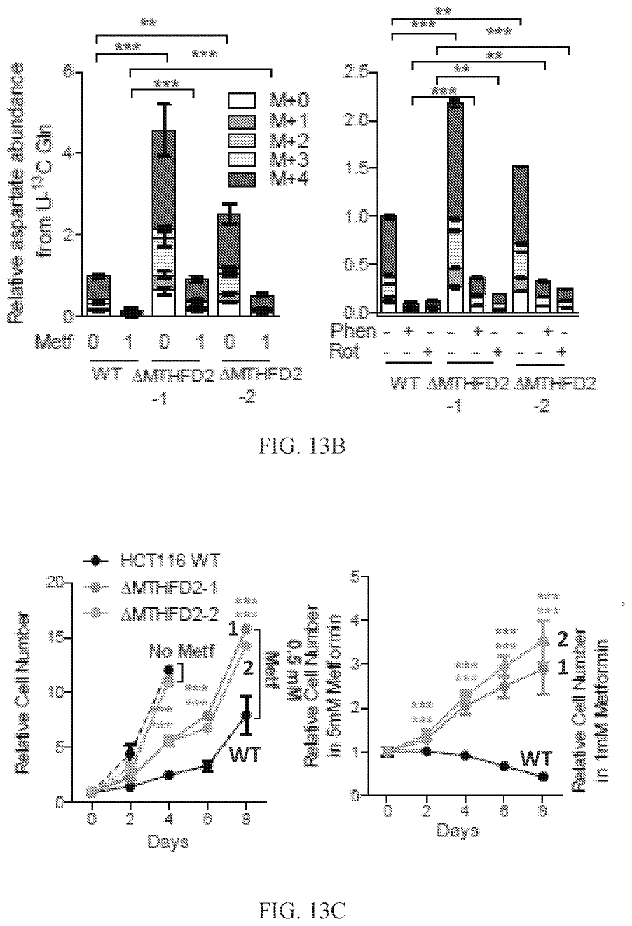

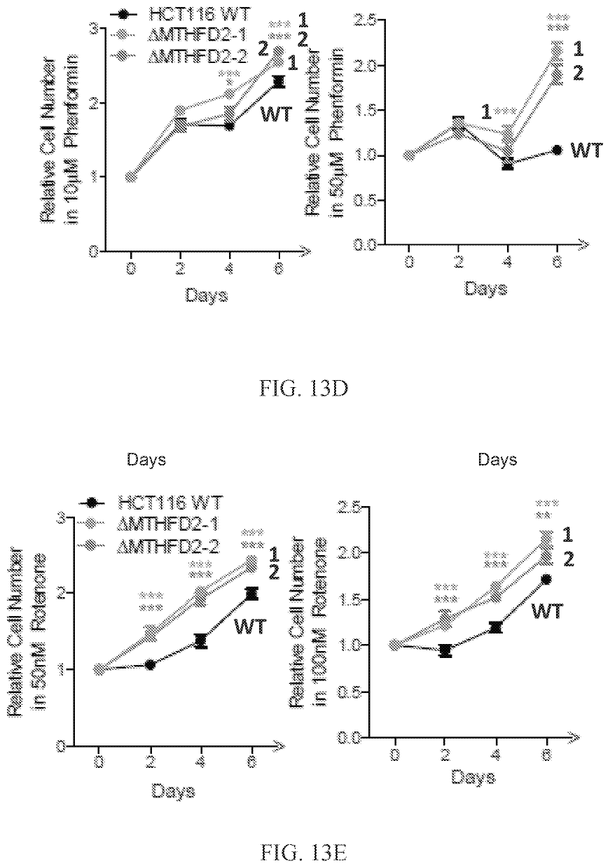

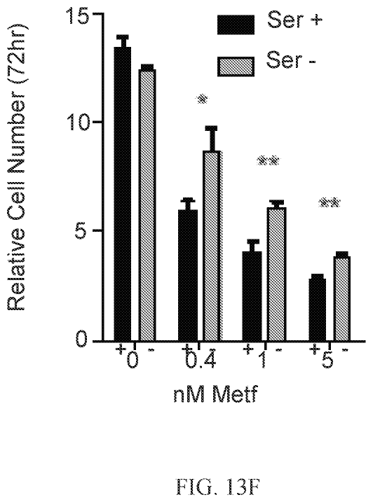

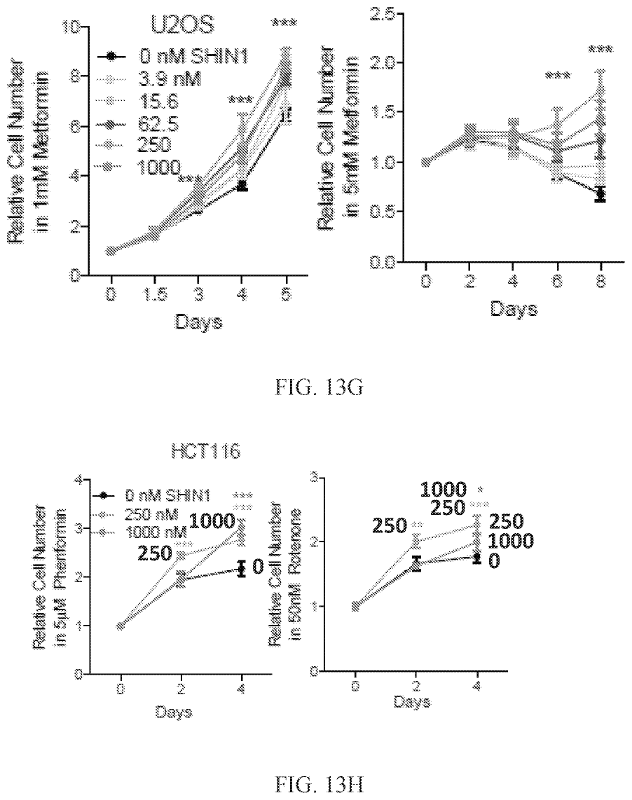

This improved capacity for metabolic homeostasis resulted in faster growth of MTHFD2 knockout cells in the presence of pharmacological complex I inhibition, especially at high drug concentrations (FIGS. 5E, 13A). Similarly, removal of serine from the tissue culture medium rendered cell growth partially resistant to metformin (FIG. 13B). To confirm that mitochondrial serine catabolism can be anti-proliferative when respiration is impaired, cells were treated with the small molecule inhibitor of serine catabolism, SHIN1. While SHIN1 decreased cell proliferation in standard tissue culture conditions (FIG. 13C), it augmented proliferation in the presence of metformin, phenformin or rotenone (FIGS. 5F, 13C, 13D, 13E). Thus, when respiration is impaired, excessive NADH generation via serine catabolism can decrease cell growth.

### Example 6. Hypoxia Also Induces Serine-Dependent NADH Production

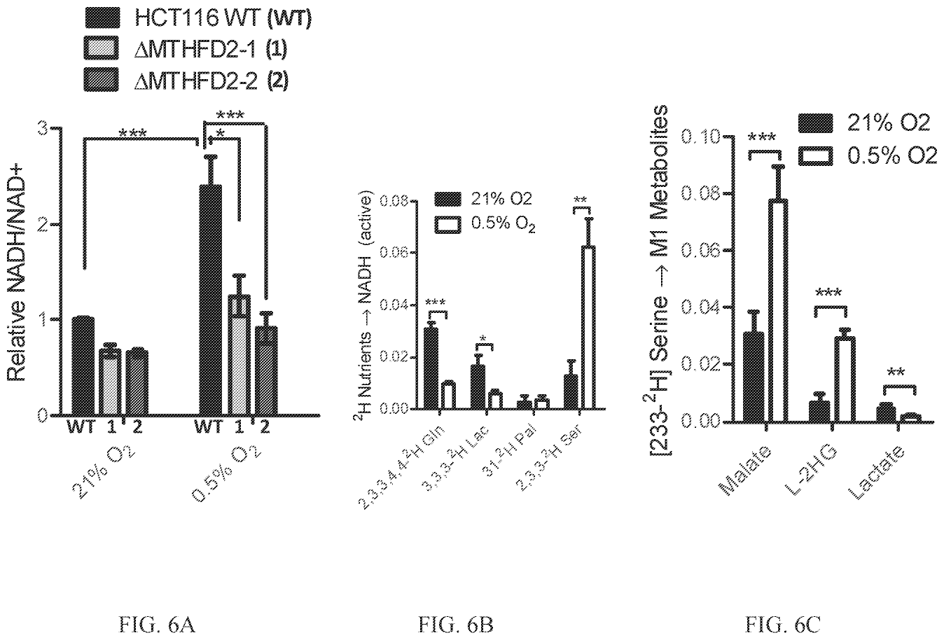

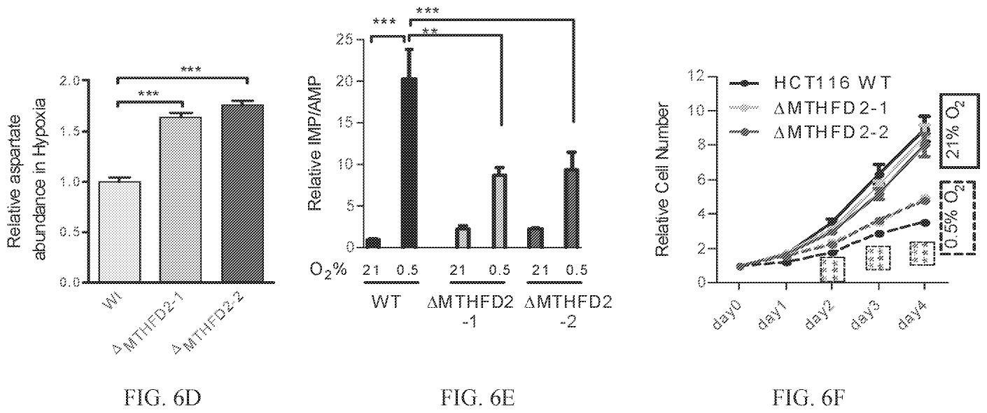

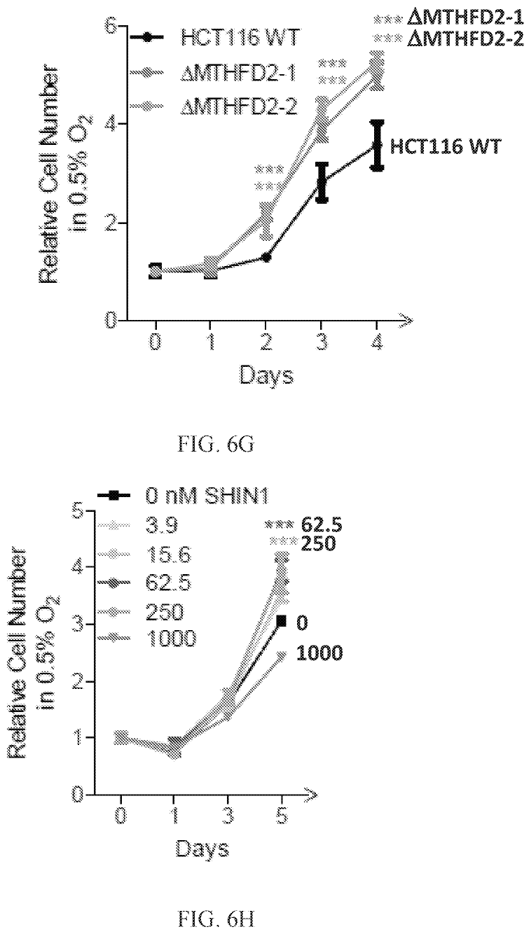

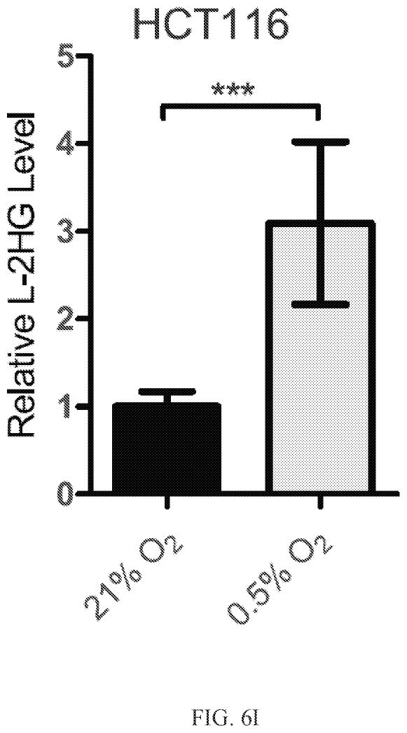

For convenience complex I inhibition was relied on to induce respiration impairment. While metformin is a very important pharmaceutical, physiologically, hypoxia is the most important cause of respiration impairment. Hypoxia resulted in an elevated fractional contribution of serine to NADH (FIG. 6B). Examination of compartmentalized downstream NADH products revealed that the NADH was produced from serine in mitochondria, as label was transferred to malate and L-2 hydroxyglutarate (which can be made mitochondrially), but not lactate (which is produced from NADH in the cytosol) (FIGS. 6C and 6I).

Similar to metformin treatment, hypoxia induced an increased NADH/NAD ratio, which was blunted by MTHFD2 knockout (FIG. 6A). Correspondingly, MTHFD2 knockout cells maintained a higher aspartate concentration, lower IMP/AMP ratio and grew faster than wild-type cells in hypoxia (FIGS. 6D-6G). Low doses of SHIN1 also modestly increased cell growth in hypoxia (FIG. 6H). Thus, the observations using metformin generalize also to respiration impairment induced by hypoxia.

### Example 7. Serine's Contribution to NADH is Enhanced by Leigh Syndrome Mutations

Another cause of respiration impairment is genetic deficiencies of the electron transport chain. In humans, such deficiencies manifest in diseases including Leigh syndrome, a severe neurological disorder that results in cognitive and motor impairment, typically starting during the first year of life. Leigh Syndrome is caused by diversity of nuclear and mitochondrial DNA mutations that result in impaired respiratory chain function. Fibroblasts from Leigh Syndrome patients and controls were compared and an increased NADH/NAD ratio and ²H-serine contribution to NADH and malate in the patient fibroblasts were observed (FIGS. 7A-7C).

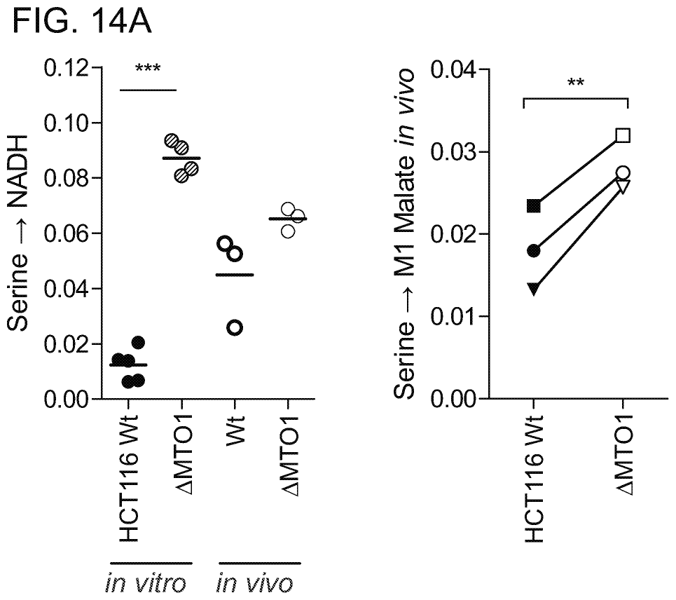

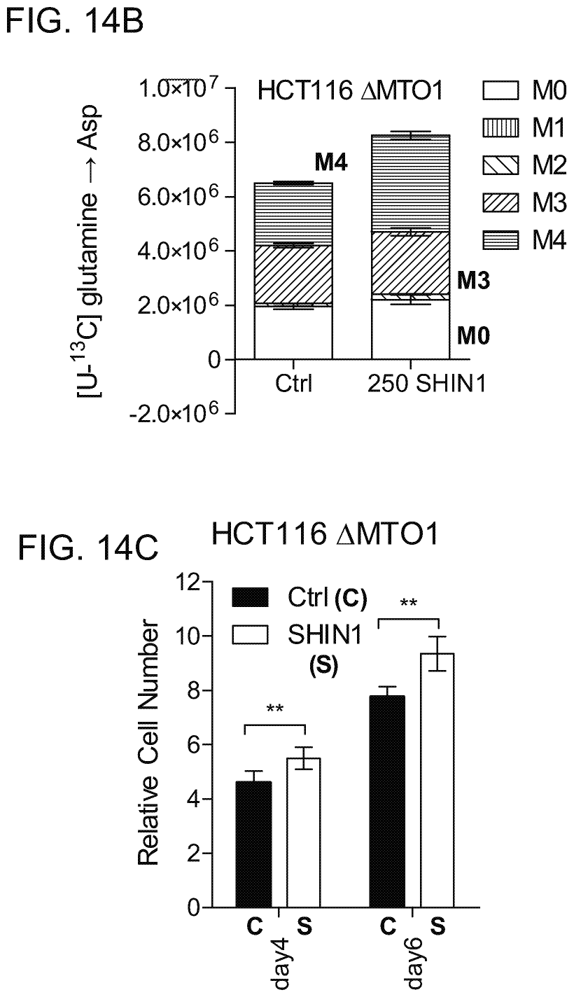

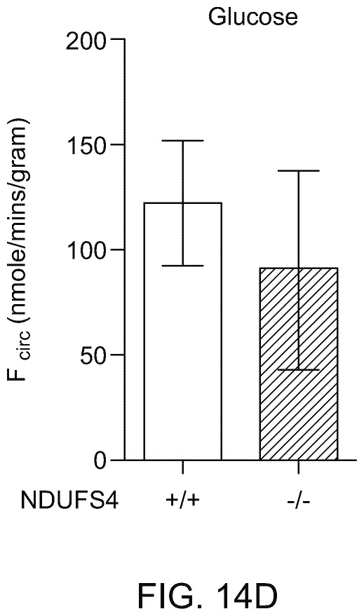

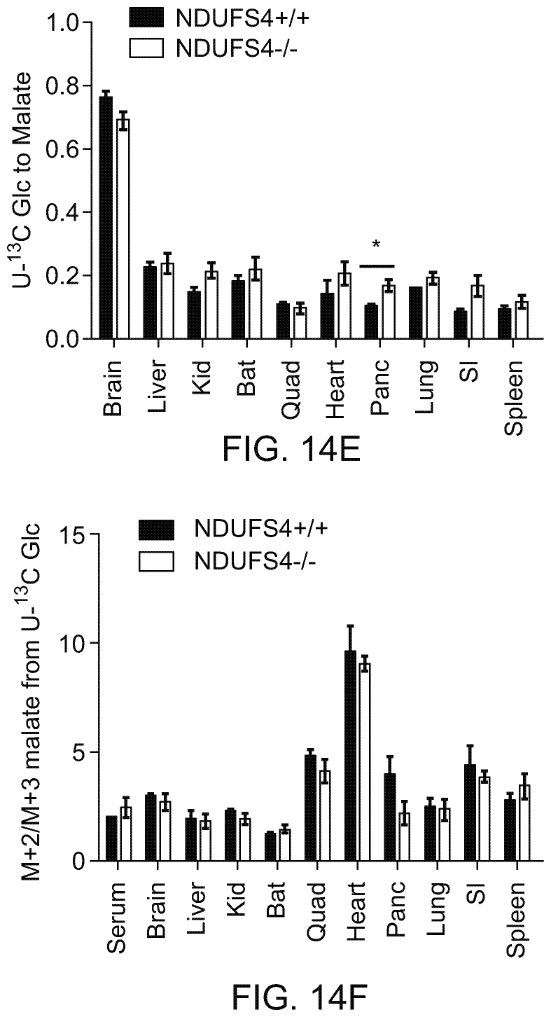

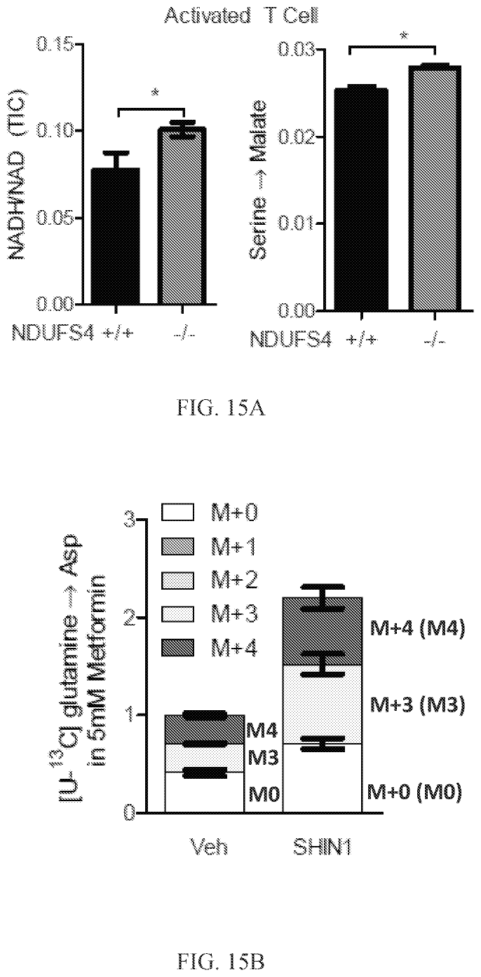

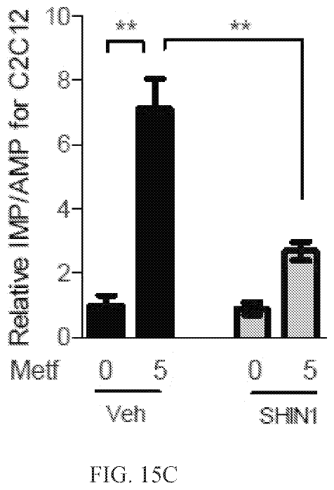

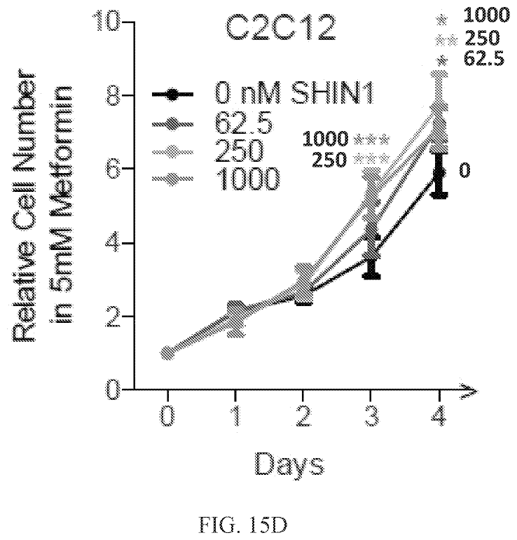

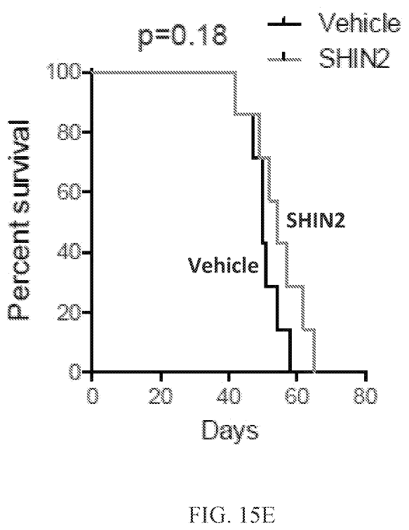

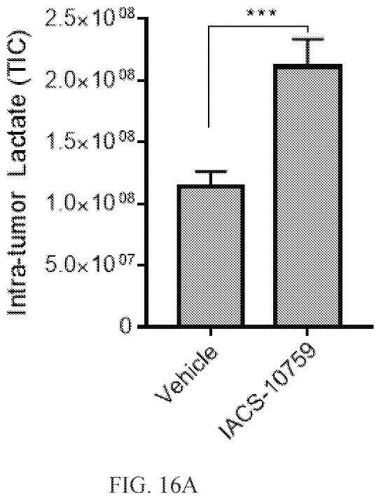

One of the frequently mutated genes in mitochondrial diseases is MTO1, an enzyme involved in mitochondrial tRNA modification and thereby respiratory chain translation. In genetically engineered colon cancer cells lacking this gene, an increased serine contribution to NADH both in cell culture and as xenograft tumors was observed (FIG. 14A). Moreover, pharmacological blockade of mitochondrial serine metabolism increased these cells' aspartate level and growth rate (FIGS. 14B-14C).

Mutations in the complex I accessory subunit NDUFS4 also cause mitochondrial deficiency. NDUFS4−/− mice have emerged as a useful model for mitochondrial diseases including Leigh Syndrome. Consistent with defective oxidative metabolic capacity and decreased whole-body oxygen uptake, NDUFS4−/− mice manifested lower circulatory turnover flux of glutamine. The fractional contribution of carbohydrate (as measured by TCA labeling from U-13C-glucose) and U-¹³Cglutamine to TCA metabolism was not dramatically altered in the complex I-deficient mice, with the notable exception of a decreased glutamine TCA contribution in pancreas and liver. In addition, TCA isotope labeling patterns did not shift much, with no evidence for decreased PDH flux (as measured by M+2/M+3 malate labeling from U-¹³C-glucose) or increased reductive carboxylation (as measured by increased M+3/M+4 malate labeling from U-¹³C-glutamine). Infusion of [2,3,3-²H]serine revealed increased ²H-malate labeling in the mutant mice, which was most evident in the pancreas but also occurred with incomplete penetrance in liver and spleen, the other two major organs to use serine as an NADH supplier (FIGS. 1B, 7D). Upon in vitro stimulation, T cells from NDUFS4−/− mice also showed increased NADH/NAD and serine contribution to NADH. Thus, mitochondrial genetic mutations enhance serine's contribution to NADH both in vitro and in vivo.

One of the frequently observed symptoms for the neurological disorder in NDUFS4−/− mice is limb clasping, which reflects mitochondrial disease progression). As cancer cells are partially protected from complex I inhibition by serine catabolism inhibition, whether such protection extends to muscle, where it could potentially be therapeutically advantageous, was probed. Indeed, C2C12 myoblasts better tolerated metformin in the presence of pharmacological serine catabolism inhibitor (SHIN1). A dual SHMT1/2 inhibitor with improved pharmacokinetics, SHIN2, was tested for whether it could decrease NADH production from serine in vivo. Single intraperitoneal injection of SHIN2 (200 mg/kg) before tracer infusion blocked most malate labeling from [2,3,3-2H]serine (FIG. 7E). It was then tested whether SHIN2 could partially ameliorate the long-term effects of complex I deficiency. Although not significantly altering survival, treatment of NDUFS4−/− mice with SHIN2 (50 mg/kg b.i.d. continuously starting from postnatal day 25±4) delayed pathological clasping behavior (FIG. 7F). Thus, serine catabolism can contribute to mitochondrial disease-induced spasticity in vivo.

### Example 8. KDG 112 (SHIN2) Therapy of Mitochondrial Disorder

Based on the finding that targeting the mitochondrial serine catabolic pathway could ameliorate redox stress induced by respiration impairment or hypoxia, the potential for KDG112, a novel drug targeting SHMT enzymatic activity, to treat patients suffering from mitochondrial diseases was explored. To this end, a well characterized mitochondrial diseases mice model NDUFS4−/− was employed.

Mice heterozygous for the deleted Ndufs4 allele (KO mice) are indistinguishable from wild-type mice. By postnatal day 21 (P21), most Ndufs4 homozygous mice are smaller than normal and begin to lose their body hair; however, their hair generally grows back during the next hair-growth cycle. KO mice reached a maximum body weight of ˜15 g at ˜P30. Prior to P30, the KO mice are healthy, and they groom themselves, eat normally, responded to novel objects, socialize, and generally behave similar to control mice. They have normal locomotor activity during both day and night until ˜P35, after which they gradually became lethargic.

Treatment strategy: Mice were grouped as vehicle treatment group and KDG112 treatment group. KDG112 was dissolved in 20% hydroxypropyl cyclodextrin to a final concentration of 5 mg/ml. Mice were dosed B.I.D at 50 mg/kg ip. Mice weight and body temperature are recorded, and body condition score were given according to the chart below.

| Motor Skills: | | Score |
|---|---|---|
| Locomotor activity | ≥2 Quadrants | 0 |
| | 0 or 1 Quadrant | 1 |
| Righting ability | No problem | 0 |
| | Lands on side/back | 1 |
| Physiological: | Weight Increase | 0 |
| | No increase | 1 |
| | Increase/decrease/erratic | 2 |
| General | | |
| Gasp, heave, tremor, shiver | Any occurrence in 1 min | 1 |
| Trunk curl, hind limb clasp | Any attempt in 10 s | 1 |
| Touch response | Moderate, brisk withdrawal | 0 |
| | No response, slight | 1 |
| | withdrawal | |

Humane end point: Mice were euthanized upon loss of more than 20% of its peak weight

The data show that KDG112 treated mice have a lower score compared to the vehicle treated group, and higher body temperature, better locomotor activity, and delayed symptoms of hind limb clasp.

Two pairs of mice have been analyzed with each one treated with vehicle or KDG112.

First pair of pups from same breeding pair: Date of Birth Dec. 27, 2018. Treatment start on Jan. 25, 2019 (P29). At day 49 (P49), vehicle treatment mouse already has obvious hind limb clasping, and cannot walk properly on the cage edge test. Its body temperature is relatively lower than KDG112 treated mouse, which can control its leg normally as wild type, and can walk freely on the edge test. 10 days later, KDG112 treated mouse also gradually develops neurological diseases symptoms such as hind limb clasping. At day 63, the vehicle treatment mouse has already lost 20% of its peak weight, and is euthanized. KDG112 treated mouse still maintains its weight.

Second pair of pups from same breeding pair: Date of Birth Jan. 3, 2019. Treatment start on Jan. 31, 2019 (P28), and at day 38, vehicle treatment mouse has already developed severe symptoms indicating neurological problems. The mouse is not able to right itself, shivers and has trunk tremor when lifted up. The mouse is not able to walk around in new cages. The KDG112 treated mouse behaves normally at that stage. At day 47, the vehicle-treated mouse lost around 20% of its peak weight, and is euthanized. At day 47, KDG112 treated mouse also develops symptoms such as hind limb clasping and low tendency to walk in new cages.

Discussion: NAD is essential for metabolism. Oxidative energy production correlates with flux through mitochondrial NADH. Accordingly, there has been extensive research on NADH sources in different physiological and pathological contexts (Chen et al., 2018; Gaude et al., 2018; Guarente, 2008; McKenna et al., 2006; Owen et al., 2002; Solaini et al., 2010; Yang et al., 2014; Yaniv et al., 2013). A limitation of these studies has been inferring NADH sources based on tracking carbon flows, even though the key energy-carrying functional group in NADH is actually the redox-active hydrogen. Here, NADH sources were directly measured using heavy hydrogen (deuterium), revealing that serine is a substantial NADH source.

Serine contributes to NADH independent from the TCA cycle, via folate-mediated mitochondrial serine catabolism. The enzyme SHMT2 converts serine to glycine with concomitant production of methylene-THF. The enzyme MTHFD2 then transfers electrons from methylene-THF to NAD, producing 10-formyl-THF and NADH. We prove genetically that both of these enzymes are essential to serine dependent NADH production. Because MTHFD2 is resistant to product inhibition by NADH, the activity of these enzymes persists when respiration is impaired. Accordingly, when respiration is impaired, even though the absolute flux through serine catabolism likely does not increase, the fraction of NADH made by serine catabolism rises.

Is this flux physiologically significant? In normal physiological conditions, 4% of NADH in pancreas is labeled by 2H-serine infusion. The real contribution of serine catabolism is, however, larger, as labeling is limited by the deuterium kinetic isotope effect (slower reactivity of deuterated, compared to unlabeled, methylene-THF) and by 1H-2H exchange between NADH and water (Ducker et al., 2016; Schmidt et al., 2000; Wiberg, 1955; Zhang et al., 2017). The magnitude of both of these effects is on the order of 2-fold (perhaps substantially larger for 1H-2H exchange), suggesting that ˜20% or more of pancreas NADH is made by serine catabolism, with serine's contribution less in other tissues.

When respiration is impaired, the importance of serine catabolism as NADH source further increases. This reflects shutting off of other routes of NADH production, which in cell culture occurs in a hierarchical manner. Relatively modest elevations in NADH inhibit serine synthesis from glucose, pyruvate dehydrogenase, and/or NADH-dependent isocitrate dehydrogenase, preventing oxidation of pyruvate, lactate, and glucose. Further elevations re-direct glutamine metabolism, from oxidative TCA turning to reductive carboxylation. Yet stronger NADH elevations decrease fatty acid oxidation. These mechanisms collectively help to prevent pathological NADH elevation. Interestingly, in complex I deficient mice, overall glutamine flux was decreased, but the relative contribution of glucose and glutamine to TCA did not shift markedly in most tissues, nor was there evidence for dramatic shifts in their handling within the TCA cycle. This is consistent with a requirement to maintain mass balance at the whole-body level: ultimately, the contents of ingested food must be oxidized. This contrasts with cultured cells, which can pick their preferred substrates from diverse media constituents.

Not only does serine catabolism persist when respiration is impaired, certain regulatory mechanisms actively promote it. Hypoxia induces SHMT2 via MYC and HIF, which can also upregulate the serine synthesis pathway and other mitochondrial 1C enzymes (MTHFD2, MTHFD1L). It has been found that this induction of serine catabolism is, at least at the cellular level, maladaptive: when respiration is impaired, genetic or pharmacological inhibition of serine catabolism enhances cell growth. Further research is merited to determine whether this is a quirk of evolution or reflects a higher organismal objective, such as to prevent the proliferation of respiration deficient cells or to attain other products of 1C metabolism at a poorly vascularized wound site.

In mitochondrial diseases, additional physiological signaling mechanisms result in counter-productive responses. Mitochondrial deficiency turns on amino acid starvation response signaling by ATF4, likely via aspartate depletion. For unclear reasons, it also activates mTOR. Both ATF4 and mTOR promote MTHFD2 expression and thus mitochondrial serine catabolism. The activation of mTOR in mitochondrial disorders is maladaptive, as rapamycin improves mitochondrial myopathy and prolongs life span. Moreover, it was found that serine catabolism inhibitor modestly ameliorates disease progression in the NDUFS4 Leigh Syndrome mouse model.

Clinically, there is no curative therapy for mitochondrial diseases (Murayama et al., 2019; Suomalainen and Battersby, 2018). “Mitochondrial cocktails” of vitamins and cofactors including coenzyme Q are widely used but have no proven clinical efficacy. Massive pyruvate intake offers a non-mitochondrial pathway to NADH oxidation, but is far from a robust solution. Chronic hypoxia slows disease progression in mouse, by suppressing reactive oxygen species, but is hard to implement clinically. Each of these strategies might work better when combined with inhibition of serine catabolism, to decrease serine-driven NADH accumulation.

More generally, it may be possible to use the serine pathway to modulate NADH status. For example, one can envision feeding excess serine and thereby overloading hypoxic tumors with NADH. Conversely, serine catabolism inhibitors might mitigate NADH buildup during ischemic events, reducing suffering in diseases of chronic vascular insufficiency and/or enabling safer reperfusion in acute events.

### REFERENCES

Ben-Sahra, I., Hoxhaj, G., Ricoult, S. J. H., Asara, J. M., and Manning, B. D. (2016). mTORC1 induces purine synthesis through control of the mitochondrial tetrahydrofolate cycle. Science 351, 728-733.
Birsoy, K., Wang, T., Chen, W. W., Freinkman, E., Abu-Remaileh, M., and Sabatini, D. M. (2015). An Essential Role of the Mitochondrial Electron Transport Chain in Cell Proliferation Is to Enable Aspartate Synthesis. Cell 162, 540-551.
Chabner, B. A., and Roberts, T. G., Jr. (2005). Timeline: Chemotherapy and the war on cancer. Nature reviews Cancer 5, 65-72.
Chandel, N. S., Maltepe, E., Goldwasser, E., Mathieu, C. E., Simon, M. C., and Schumacker, P. T. (1998).
Mitochondrial reactive oxygen species trigger hypoxia-induced transcription. Proc Natl Acad Sci USA 95, 11715-11720.
Chen, Q. Y., Kirk, K., Shurubor, Y. I., Zhao, D. Z., Arreguin, A. J., Shahi, I., Valsecchi, F., Primiano, G., Calder, E. L., Carelli, V., et al. (2018). Rewiring of Glutamine Metabolism Is a Bioenergetic Adaptation of Human Cells with Mitochondrial DNA Mutations. Cell metabolism 27, 1007-+.
Darin, N., Oldfors, A., Moslemi, A. R., Holme, E., and Tulinius, M. (2001). The incidence of mitochondrial encephalomyopathies in childhood: clinical features and morphological, biochemical, and DNA abnormalities. Annals of neurology 49, 377-383.
DeBerardinis, R. J., Lum, J. J., Hatzivassiliou, G., and Thompson, C. B. (2008). The biology of cancer: Metabolic reprogramming fuels cell growth and proliferation. Cell metabolism 7, 11-20.
Diehl, F. F., Lewis, C. A., Fiske, B. P., and Vander Heiden, M. G. (2019). Cellular redox state constrains serine synthesis and nucleotide production to impact cell proliferation. Nature metabolism 1, 861-867.
Du, D., Tan, L., Wang, Y., Peng, B., Weinstein, J. N., Wondisford, F. E., Su, X., and Lorenzi, P. L. (2019).
ElemCor: accurate data analysis and enrichment calculation for high-resolution LC-MS stable isotope labeling experiments. BMC bioinformatics 20, 89.
Ducker, G. S., Chen, L., Morscher, R. J., Ghergurovich, J. M., Esposito, M., Teng, X., Kang, Y., and Rabinowitz, J. D. (2016). Reversal of Cytosolic One-Carbon Flux Compensates for Loss of the Mitochondrial Folate Pathway. Cell metabolism 24, 640-641.
Ducker, G. S., Ghergurovich, J. M., Mainolfi, N., Suri, V., Jeong, S. K., Hsin-Jung Li, S., Friedman, A., Manfredi, M. G., Gitai, Z., Kim, H., et al. (2017). Human SHMT inhibitors reveal defective glycine import as a targetable metabolic vulnerability of diffuse large B-cell lymphoma. Proc Natl Acad Sci USA 114, 11404-11409.
El-Mir, M. Y., Nogueira, V., Fontaine, E., Averet, N., Rigoulet, M., and Leverve, X. (2000).
Dimethylbiguanide inhibits cell respiration via an indirect effect targeted on the respiratory chain complex I. J Biol Chem 275, 223-228.
Fakruddin, M., Wei, F. Y., Suzuki, T., Asano, K., Kaieda, T., Omori, A., Izumi, R., Fujimura, A., Kaitsuka, T., Miyata, K., et al. (2018). Defective Mitochondrial tRNA Taurine Modification Activates Global Proteostress and Leads to Mitochondrial Disease. Cell Rep 22, 482-496.
Ferrari, M., Jain, I. H., Goldberger, O., Rezoagli, E., Thoonen, R., Chen, K. H., Sosnovik, D. E., Scherrer-Crosbie, M., Mootha, V. K., and Zapol, W. M. (2017). Hypoxia treatment reverses neurodegenerative disease in a mouse model of Leigh syndrome. P Natl Acad Sci USA 114, E4241-E4250.
Fujii, T., Nozaki, F., Saito, K., Hayashi, A., Nishigaki, Y., Murayama, K., Tanaka, M., Koga, Y., Hiejima, I., and Kumada, T. (2014). Efficacy of pyruvate therapy in patients with mitochondrial disease: A semiquantitative clinical evaluation study. Mol Genet Metab 112, 133-138.
Fullerton, M. D., Galic, S., Marcinko, K., Sikkema, S., Pulinilkunnil, T., Chen, Z. P., O'Neill, H. M., Ford, R. J., Palanivel, R., O'Brien, M., et al. (2013). Single phosphorylation sites in Acc1 and Acc2 regulate lipid homeostasis and the insulin-sensitizing effects of metformin. Nat Med 19, 1649-1654.
Gameiro, P. A., Yang, J. J., Metelo, A. M., Perez-Carro, R., Baker, R., Wang, Z. W., Arreola, A., Rathmell, W. K., Olumi, A., Lopez-Larrubia, P., et al. (2013). In Vivo HIF-Mediated Reductive Carboxylation Is Regulated by Citrate Levels and Sensitizes VHL-Deficient Cells to Glutamine Deprivation. Cell Metab 17, 372-385.
Gaude, E., Schmidt, C., Gammage, P. A., Dugourd, A., Blacker, T., Chew, S. P., Saez-Rodriguez, J., O'Neill, J. S., Szabadkai, G., Minczuk, M., et al. (2018). NADH Shuttling Couples Cytosolic Reductive Carboxylation of Glutamine with Glycolysis in Cells with Mitochondrial Dysfunction. Mol Cell 69, 581-593 e587.
Gregory, J. F., 3rd, Cuskelly, G. J., Shane, B., Toth, J. P., Baumgartner, T. G., and Stacpoole, P. W. (2000).
Primed, constant infusion with [2H3]serine allows in vivo kinetic measurement of serine turnover, homocysteine remethylation, and transsulfuration processes in human one-carbon metabolism. Am J Clin Nutr 72, 1535-1541.
Guarente, L. (2008). Mitochondria—a nexus for aging, calorie restriction, and sirtuins?Cell 132, 171-176.
Heng, J. S., Rattner, A., Stein-O'Brien, G. L., Winer, B. L., Jones, B. W., Vernon, H. J., Goff, L. A., and Nathans, J. (2019). Hypoxia tolerance in the Norrin-deficient retina and the chronically hypoxic brain studied at single-cell resolution. P Natl Acad Sci USA 116, 9103-9114.
Herbig, K., Chiang, E. P., Lee, L. R., Hills, J., Shane, B., and Stover, P. J. (2002). Cytoplasmic serine hydroxymethyltransferase mediates competition between folate-dependent deoxyribonucleotide and Sadenosylmethionine biosyntheses. J Biol Chem 277, 38381-38389.
Hui, S., Ghergurovich, J. M., Morscher, R. J., Jang, C., Teng, X., Lu, W., Esparza, L. A., Reya, T., Le, Z., Yanxiang Guo, J., et al. (2017). Glucose feeds the TCA cycle via circulating lactate. Nature 551, 115-118.
Hunter, R. W., Hughey, C. C., Lantier, L., Sundelin, E. I., Peggie, M., Zeqiraj, E., Sicheri, F., Jessen, N., Wasserman, D. H., and Sakamoto, K. (2018). Metformin reduces liver glucose production by inhibition of fructose-1-6-bisphosphatase. Nat Med 24, 1395-+.
Jain, I. H., Zazzeron, L., Goldberger, O., Marutani, E., Wojtkiewicz, G. R., Ast, T., Wang, H., Schleifer, G., Stepanova, A., Brepoels, K., et al. (2019). Leigh Syndrome Mouse Model Can Be Rescued by Interventions that Normalize Brain Hyperoxia, but Not HIF Activation. Cell Metab 30, 824-+.
Jain, I. H., Zazzeron, L., Goli, R., Alexa, K., Schatzman-Bone, S., Dhillon, H., Goldberger, O., Peng, J., Shalem, O., Sanjana, N. E., et al. (2016). Hypoxia as a therapy for mitochondrial disease. Science 352, 54-61.
Johnson, S. C., Yanos, M. E., Kayser, E. B., Quintana, A., Sangesland, M., Castanza, A., Uhde, L., Hui, J., Wall, V. Z., Gagnidze, A., et al. (2013). mTOR inhibition alleviates mitochondrial disease in a mouse model of Leigh syndrome. Science 342, 1524-1528.
Kalucka, J., Bierhansl, L., Conchinha, N. V., Missiaen, R., Elia, I., Bruning, U., Scheinok, S., Treps, L., Cantelmo, A. R., Dubois, C., et al. (2018). Quiescent Endothelial Cells Upregulate Fatty Acid (beta-Oxidation for Vasculoprotection via Redox Homeostasis. Cell metabolism 28, 881-+.
Kerr, D. S. (2013). Review of clinical trials for mitochondrial disorders: 1997-2012. Neurotherapeutics 10, 307-319.
Khan, N. A., Nikkanen, J., Yatsuga, S., Jackson, C., Wang, L. Y., Pradhan, S., Kivela, R., Pessia, A., Velagapudi, V., and Suomalainen, A. (2017). mTORC1 Regulates Mitochondrial Integrated Stress Response and Mitochondrial Myopathy Progression. Cell metabolism 26, 419-+.
Kremer, J. M. (1994). The mechanism of action of methotrexate in rheumatoid arthritis: the search continues. The Journal of rheumatology 21, 1-5.
Liu, L., Shah, S., Fan, J., Park, J. O., Wellen, K. E., and Rabinowitz, J. D. (2016a). Malic enzyme tracers reveal hypoxia-induced switch in adipocyte NADPH pathway usage. Nature chemical biology 12, 345-352.
Liu, X. J., Romero, I. L., Litchfield, L. M., Lengyel, E., and Locasale, J. W. (2016b). Metformin Targets Central Carbon Metabolism and Reveals Mitochondrial Requirements in Human Cancers. Cell metabolism 24, 728-739.
Lu, W. Y., Wang, L., Chen, L., Hui, S., and Rabinowitz, J. D. (2018). Extraction and Quantitation of Nicotinamide Adenine Dinucleotide Redox Cofactors. Antioxid Redox Sign 28, 167-179.
Lundsgaard, A. M., Fritzen, A. M., and Kiens, B. (2018). Molecular Regulation of Fatty Acid Oxidation in Skeletal Muscle during Aerobic Exercise. Trends Endocrin Met 29, 18-30.
Lunt, S. Y., and Vander Heiden, M. G. (2011). Aerobic Glycolysis: Meeting the Metabolic Requirements of Cell Proliferation. Annu Rev Cell Dev Bi 27, 441-464.
Madiraju, A. K., Erion, D. M., Rahimi, Y., Zhang, X. M., Braddock, D. T., Albright, R. A., Prigaro, B. J., Wood, J. L., Bhanot, S., MacDonald, M. J., et al. (2014). Metformin suppresses gluconeogenesis by inhibiting mitochondrial glycerophosphate dehydrogenase. Nature 510, 542-+.
Madiraju, A. K., Qiu, Y., Perry, R. J., Rahimi, Y., Zhang, X. M., Zhang, D. Y., Camporez, J. P. G., Cline, G. W., Butrico, G. M., Kemp, B. E., et al. (2018). Metformin inhibits gluconeogenesis via a redox-dependent mechanism in vivo. Nat Med 24, 1384-+. Martinez-Reyes, I., and Chandel, N. S. (2014). Mitochondrial One-Carbon Metabolism Maintains Redox Balance during Hypoxia. Cancer discovery 4, 1371-1373.
McKenna, M. C., Waagepetersen, H. S., Schousboe, A., and Sonnewald, U. (2006). Neuronal and astrocytic shuttle mechanisms for cytosolic-mitochondrial transfer of reducing equivalents: Current evidence and pharmacological tools. Biochem Pharmacol 71, 399-407.
Millard, P., Letisse, F., Sokol, S., and Portais, J. C. (2012). IsoCor: correcting MS data in isotope labeling experiments. Bioinformatics 28, 1294-1296.
Mullen, A. R., Wheaton, W. W., Jin, E. S., Chen, P. H., Sullivan, L. B., Cheng, T., Yang, Y. F., Linehan, W. M., Chandel, N. S., and DeBerardinis, R. J. (2012). Reductive carboxylation supports growth in tumour cells with defective mitochondria. Nature 481, 385-U171.
Murayama, K., Shimura, M., Liu, Z., Okazaki, Y., and Ohtake, A. (2019). Recent topics: the diagnosis, molecular genesis, and treatment of mitochondrial diseases. J Hum Genet 64, 113-125.
Neinast, M. D., Jang, C., Hui, S., Murashige, D. S., Chu, Q., Morscher, R. J., Li, X., Zhan, L., White, E., Anthony, T. G., et al. (2019). Quantitative Analysis of the Whole-Body Metabolic Fate of Branched-Chain Amino Acids. Cell metabolism 29, 417-429 e414.
Nikkanen, J., Forsstrom, S., Euro, L., Paetau, I., Kohnz, R. A., Wang, L. Y., Chilov, D., Viinamaki, J., Roivainen, A., Marjamaki, P., et al. (2016). Mitochondrial DNA Replication Defects Disturb Cellular dNTP Pools and Remodel One-Carbon Metabolism. Cell Metab 23, 635-648.
Owen, M. R., Doran, E., and Halestrap, A. P. (2000). Evidence that metformin exerts its anti-diabetic effects through inhibition of complex 1 of the mitochondrial respiratory chain. Biochem J 348, 607-614.
Owen, O. E., Kalhan, S. C., and Hanson, R. W. (2002). The key role of anaplerosis and cataplerosis for citric acid cycle function. J Biol Chem 277, 30409-30412.
Papandreou, I., Cairns, R. A., Fontana, L., Lim, A. L., and Denko, N. C. (2006). HIF-1 mediates adaptation to hypoxia by actively downregulating mitochondrial oxygen consumption. Cell Metab 3, 187-197.
Pfeffer, G., Majamaa, K., Turnbull, D. M., Thorburn, D., and Chinnery, P. F. (2012). Treatment for mitochondrial disorders. Cochrane Database Syst Rev, CD004426.
Saito, K., Kimura, N., Oda, N., Shimomura, H., Kumada, T., Miyajima, T., Murayama, K., Tanaka, M., and Fujii, T. (2012). Pyruvate therapy for mitochondrial DNA depletion syndrome. Bba-Gen Subjects 1820, 632-636.
Samanta, D., Park, Y., Andrabi, S. A., Shelton, L. M., Gilkes, D. M., and Semenza, G. L. (2016). PHGDH Expression Is Required for Mitochondrial Redox Homeostasis, Breast Cancer Stem Cell Maintenance, and Lung Metastasis. Cancer Res 76, 4430-4442.
Schmidt, A., Wu, H. P., MacKenzie, R. E., Chen, V. J., Bewly, J. R., Ray, J. E., Toth, J. E., and Cygler, M. (2000).
Structures of three inhibitor complexes provide insight into the reaction mechanism of the human methylenetetrahydrofolate dehydrogenase/cyclohydrolase. Biochemistry-Us 39, 6325-6335.
Solaini, G., Baracca, A., Lenaz, G., and Sgarbi, G. (2010). Hypoxia and mitochondrial oxidative metabolism. Biochimica et biophysica acta 1797, 1171-1177.
Su, X., Lu, W., and Rabinowitz, J. D. (2017). Metabolite Spectral Accuracy on Orbitraps. Analytical chemistry 89, 5940-5948.
Sullivan, L. B., Gui, D. Y., Hosios, A. M., Bush, L. N., Freinkman, E., and Vander Heiden, M. G. (2015).
Supporting Aspartate Biosynthesis Is an Essential Function of Respiration in Proliferating Cells. Cell 162, 552-563.
Sullivan, L. B., Luengo, A., Danai, L. V., Bush, L. N., Diehl, F. F., Hosios, A. M., Lau, A. N., Elmiligy, S., Malstrom, S., Lewis, C. A., et al. (2018). Aspartate is an endogenous metabolic limitation for tumour growth. Nat Cell Biol 20, 782-+.
Suomalainen, A., and Battersby, B. J. (2018). Mitochondrial diseases: the contribution of organelle stress responses to pathology. Nat Rev Mol Cell Biol 19, 77-92.
Titov, D. V., Cracan, V., Goodman, R. P., Peng, J., Grabarek, Z., and Mootha, V. K. (2016). Complementation of mitochondrial electron transport chain by manipulation of the NAD(+)/NADH ratio. Science 352, 231-235.
Tyynismaa, H., Carroll, C. J., Raimundo, N., Ahola-Erkkila, S., Went, T., Ruhanen, H., Guse, K., Hemminki, A., Peltola-Mjosund, K. E., Tulkki, V., et al. (2010). Mitochondrial myopathy induces a starvation-like response. Hum Mol Genet 19, 3948-3958.
Wang, L., Xing, X., Chen, L., Yang, L., Su, X., Rabitz, H., Lu, W., and Rabinowitz, J. D. (2019). Peak Annotation and Verification Engine for Untargeted LC-MS Metabolomics. Analytical chemistry 91, 1838-1846.
Weinblatt, M. E., Coblyn, J. S., Fox, D. A., Fraser, P. A., Holdsworth, D. E., Glass, D. N., and Trentham, D. E. (1985). Efficacy of low-dose methotrexate in rheumatoid arthritis. The New England journal of medicine 312, 818-822.
Wheaton, W. W., Weinberg, S. E., Hamanaka, R. B., Soberanes, S., Sullivan, L. B., Anso, E., Glasauer, A., Dufour, E., Mutlu, G. M., Budigner, G. R. S., et al. (2014). Metformin inhibits mitochondrial complex I of cancer cells to reduce tumorigenesis. Elife 3.
Wiberg, K. B. (1955). The Deuterium Isotope Effect. Chem Rev 55, 713-743.
Wise, D. R., Ward, P. S., Shay, J. E. S., Cross, J. R., Gruber, J. J., Sachdeva, U. M., Platt, J. M., DeMatteo, R. G., Simon, M. C., and Thompson, C. B. (2011). Hypoxia promotes isocitrate dehydrogenase-dependent carboxylation of alpha-ketoglutarate to citrate to support cell growth and viability. P Natl Acad Sci USA 108, 19611-19616.
Yang, C. D., Ko, B., Hensley, C. T., Jiang, L., Wasti, A. T., Kim, J., Sudderth, J., Calvaruso, M. A., Lumata, L., Mitsche, M., et al. (2014). Glutamine Oxidation Maintains the TCA Cycle and Cell Survival during Impaired Mitochondrial Pyruvate Transport. Mol Cell 56, 414-424.
Yaniv, Y., Juhaszova, M., and Sollott, S. J. (2013). Age-related changes of myocardial ATP supply and demand mechanisms. Trends Endocrinol Metab 24, 495-505.
Ye, J., Fan, J., Venneti, S., Wan, Y. W., Pawel, B. R., Zhang, J., Finley, L. W., Lu, C., Lindsten, T., Cross, J. R., et al. (2014). Serine catabolism regulates mitochondrial redox control during hypoxia. Cancer discovery 4, 1406-1417.
Yoo, H., Antoniewicz, M. R., Stephanopoulos, G., and Kelleher, J. K. (2008). Quantifying reductive carboxylation flux of glutamine to lipid in a brown adipocyte cell line. J Biol Chem 283, 20621-20627.
Zhang, Z., Chen, L., Liu, L., Su, X., and Rabinowitz, J. D. (2017). Chemical Basis for Deuterium Labeling of Fat and NADPH. Journal of the American Chemical Society 139, 14368-14371.
Zheng, X. D., Boyer, L., Jin, M. J., Kim, Y., Fan, W. W., Bardy, C., Berggren, T., Evans, R. M., Gage, F. H., and Hunter, T. (2016). Alleviation of neuronal energy deficiency by mTOR inhibition as a treatment for mitochondria-related neurodegeneration. Elife 5.

### IACS-10759

IACS-10759 is a potent mitochondrial complex I inhibitor with efficacy in inhibiting tumor growth and inducing apoptosis in models of brain cancer and acute myeloid leukemia. Molina, J. R. et al., Nat. Med. 24, 1036-1046 (2018). Oral gavaging of IACS-10759 (10 mg/kg) to mice xenograft with HCT116 colon cancer cells increased intra-tumor lactate level and NADH/NAD ratio, reflecting its strong inhibition of tumor respiration in vivo. To check whether circulating serine contributes NADH when respiration is impaired in vivo, [2,3,3-²H]serine was infused into tumor bearing mice. [2,3,3-²H]serine tracing revealed increased ²H-malate labeling with IACS-10759 treatment, which reflects increased serine's contribution to tumor NADH.

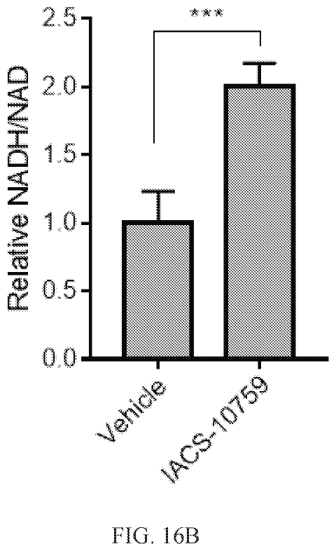

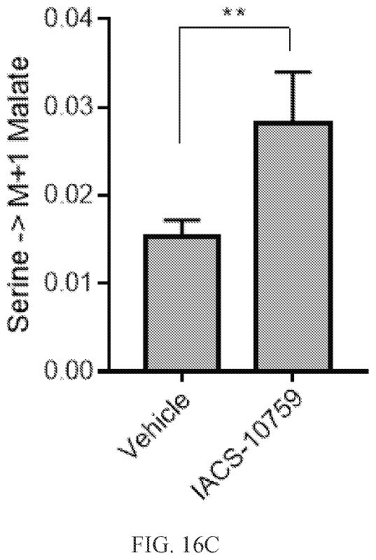

The results of the experiments with IACS-10759 are shown in FIGS. 16A-16C.

### High Serine Metformin Drinking Water

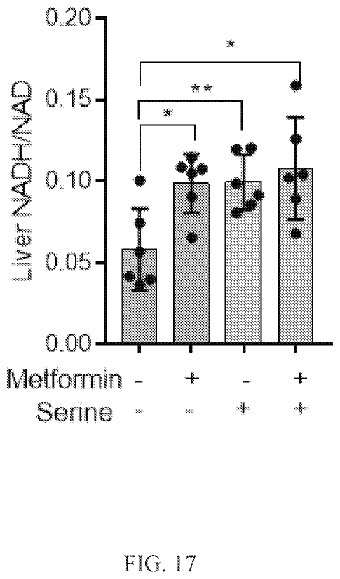

To prove whether serine/metformin can generate excess NADH in vivo, mice were supplied with high serine drinking water (5%) or metformin drinking water (0.25%) or high serine and metformin drinking water. The metabolomics analysis showed that serine and/or metformin in drinking water can significantly increase NADH/NAD ratio in liver, reflecting the reductive stress caused by serine and metformin. The results of the high serine/metformin drinking water experiment are shown in FIG. 17.

Unless defined otherwise, all technical and scientific terms used herein have the same meaning as commonly understood by one of ordinary skill in the art to which this disclosure pertains.

As used herein, the indefinite articles “a” and “an” should be understood to mean “at least one” unless clearly indicated to the contrary.

The phrase “and/or”, as used herein, should be understood to mean “either or both” of the elements so conjoined, i.e., elements that are conjunctively present in some cases and disjunctively present in other cases.

It should also be understood that, unless clearly indicated to the contrary, in any methods described herein that include more than one step or act, the order of the steps or acts of the method is not necessarily limited to the order in which the steps or acts of the method are recited.

Unless otherwise indicated or otherwise evident from the context and understanding of one of ordinary skill in the art, values that are expressed as ranges can assume any specific value or subrange within the stated ranges in various embodiments, unless the context clearly dictates otherwise.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating a disorder associated with excess NADH in a subject in need thereof, comprising administering an effective amount of an inhibitor of serine metabolism to the subject.

2. A method of treating a disorder associated with excess NADH in a subject in need thereof, comprising modulating serine metabolism in the subject.

3. The method of claim 2, wherein modulating comprises inhibiting serine metabolism.

4. The method of claim 3, wherein inhibiting serine metabolism comprises administering an inhibitor of serine metabolism to the subject.

5. A method of treating a mitochondrial disorder in a subject in need thereof, comprising administering an effective amount of an inhibitor of serine metabolism to the subject, wherein the inhibitor of serine metabolism inhibits mitochondrial serine metabolism.

6. A method for treating a disease involving deficient respiration in a subject in need thereof, comprising administering a therapeutically effective amount of an inhibitor of serine metabolism to the subject.

7. A method of improving the therapeutic index of an electron transport chain inhibitor in a subject, comprising administering to the subject a therapeutically effective amount of an inhibitor of serine metabolism, wherein the subject is receiving the electron transport chain inhibitor in combination with the inhibitor of serine metabolism.

8. The method of any one of claims 1 and 4-7, wherein the inhibitor of serine metabolism is an inhibitor of serine catabolism.

9. The method of claim 8, wherein the inhibitor of serine catabolism is an SHMT inhibitor.

10. The method of claim 9, wherein the SHMT inhibitor is an SHMT2 inhibitor.

11. The method of claim 9, wherein the SHMT inhibitor is an SHMT1 inhibitor.

12. The method of claim 9, wherein the SHMT inhibitor is an SHMT2 inhibitor and an SHMT1 inhibitor.

13. The method of claim 8, wherein the inhibitor of serine catabolism is an antifolate.

14. The method of claim 8, wherein the inhibitor of serine catabolism is an MTHFD2 inhibitor.

15. The method of any one of claims 1-14, wherein the subject has received an electron transport chain inhibitor.

16. The method of any one of claims 1, 4-6 and 8-15, wherein the subject is receiving an electron transport chain inhibitor in combination with the inhibitor of serine catabolism or inhibitor of serine metabolism.

17. The method of any one of claims 7, 8, 15 and 16, wherein the electron transport chain inhibitor is metformin, phenformin, rotenone, deguelin, antimycin, oligomycin, or a combination thereof.

18. The method of any one of claims 7, 8 and 15-17, wherein the electron transport chain inhibitor is IACS-10759.

19. The method of any one of claims 1-5 and 9-18, wherein the disorder is respiration impairment.

20. The method of claim 19, wherein the respiration impairment is caused by a genetic deficiency of the electron transport chain.

21. The method of claim 19, wherein the respiration impairment is caused by Leigh syndrome.

22. The method of any one of claims 1-5 and 9-18, wherein the disorder is caused by hypoxia, an ischemic event, a tumor or impaired oxygenation due to excess adipose tissue.

23. The method of any one of claims 6 and 8-18, wherein the disease is a genetically inherited mitochondrial disorder.

24. The method of any one of claims 6 and 8-18, wherein the disease is myocardial infarction or stroke.

25. A method for improving the effectiveness of an electron transport chain inhibitor in a subject, comprising administering to the subject an effective amount of a serine supplement, wherein the subject is receiving the electron transport chain inhibitor in combination with the serine supplement.

26. The method of claim 25, wherein the subject has received an electron transport chain inhibitor.

27. The method of claim 25 or 26, wherein the electron transport chain inhibitor is metformin, phenformin, rotenone, deguelin, antimycin, oligomycin, or a combination thereof.

28. The method of any one of claims 25-27, wherein the electron transport chain inhibitor is IACS-10759.
